(19) United States
(12) Patent Application Publication
(10) Pub. No.: US 2025/0277205 A1
(43) Pub. Date: Sep. 4, 2025

(54) RECOMBINANT YEAST CELL

(71) Applicant: DANISCO US INC., Palo Alto, CA (US)

(72) Inventors: Sergio Luis Rossell-Aragort, Echt (NL); Mickel Leonardus August Jansen, Echt (NL); Ingrid Maria Vugt-Van Lutz, Echt (NL); Jozef Petrus Johannes Schmitz, Echt (NL); Evert Tjeerd Van Rij, Echt (NL); René Marcel de Jong, Echt (NL)

(21) Appl. No.: 18/578,581

(22) PCT Filed: Jul. 7, 2022
(86) PCT No.: PCT/EP2022/068916

§ 371 Date: Jan. 11, 2024

(30) Foreign Application Priority Data
Jul. 12, 2021 (EP) 21185143.1

(51) Int. Cl. C12N 9/88; C12N 9/04; C12N 9/10; C12N 9/12; C12N 9/34; C12P 7/06

(52) U.S. Cl. CPC C12N 9/88; C12N 9/0006; C12N 9/1022; C12N 9/1205; C12N 9/2428; C12P 7/06; C12Y 101/01006; C12Y 202/01001; C12Y 207/01019; C12Y 207/01029; C12Y 302/01003; C12Y 401/01039

(57) ABSTRACT

A recombinant yeast cell functionally expressing:
a) a heterologous nucleic acid sequence encoding a protein having ribulose-1,5-phosphate carboxylase/oxygenase activity (EC4.1.1.39; Rubisco), a heterologous nucleic acid sequence encoding a protein having phosphoribulokinase activity (EC2.7.1.19; PRK) and optionally one or more nucleic acid sequences encoding for molecular chaperones for the protein having ribulose-1,5-phosphate carboxylase/oxygenase activity; and
b) a nucleic acid sequence encoding a protein having transketolase activity (EC 2.2.1.1), wherein the expression of the nucleic acid sequence encoding the protein having transketolase activity is under control of a promoter (the “TKL promoter”), which TKL promoter has an anaerobic/aerobic expression ratio for the transketolase of 2 or more.

### CROSS-REFERENCE TO RELATED APPLICATIONS

This application is a U.S. National Stage Application under 35 U.S.C. § 371 of International Application No. PCT/EP2022/068916, filed Jul. 7, 2022, which claims priority to European Application No. 21185143.1, filed Jul. 12, 2021, all of which are hereby incorporated by reference in their entireties.

### FIELD OF THE INVENTION

The invention relates to a recombinant yeast cell having the ability to produce ethanol and to a method for producing ethanol wherein said yeast cell is used.

### INCORPORATION BY REFERENCE OF THE SEQUENCE LISTING

The present application is being filed with a Sequence Listing in electronic format. The Sequence Listing is provided as a file entitled IFF34055-US-PCT_SEQ_Listing.xml, created on Jul. 31, 2024, which is 134,108 bytes in size. The information in the electronic format of the Sequence Listing is incorporated by reference in its entirety.

### BACKGROUND OF THE INVENTION

Microbial fermentation processes are applied to industrial production of a broad and rapidly expanding range of chemical compounds from renewable carbohydrate feedstocks. Especially in anaerobic fermentation processes, redox balancing of the cofactor couple NADH/NAD⁺ can cause important constraints on product yields. This challenge is exemplified by the formation of glycerol as major by-product in the industrial production of—for instance—fuel ethanol by Saccharomyces cerevisiae, a direct consequence of the need to reoxidize NADH formed in biosynthetic reactions.

Ethanol production by Saccharomyces cerevisiae is currently, by volume, the single largest fermentation process in industrial biotechnology. Various approaches have been proposed to improve the fermentative properties of organisms used in industrial biotechnology by genetic modification. A major challenge relating to the stoichiometry of yeast-based production of ethanol, is that substantial amounts of NADH-dependent side-products such as glycerol are generally formed as a by-product, especially under anaerobic and oxygen-limited conditions or under conditions where respiration is otherwise constrained or absent. It has been estimated that, in typical industrial ethanol processes, up to about 4 wt. % of the sugar feedstock is converted into glycerol (Nissen et al. Yeast 16 (2000) 463-474). Under conditions that are ideal for anaerobic growth, the conversion into glycerol may even be higher, up to about 10%.

Glycerol production under anaerobic conditions is primarily linked to redox metabolism. During anaerobic growth of S. cerevisiae, sugar dissimilation occurs via alcoholic fermentation. In this process, the NADH formed in the glycolytic glyceraldehyde-3-phosphate dehydrogenase reaction is reoxidized by converting acetaldehyde, formed by decarboxylation of pyruvate to ethanol via NAD⁺-dependent alcohol dehydrogenase. The fixed stoichiometry of this redox-neutral dissimilatory pathway causes problems when a net reduction of NAD⁺ to NADH occurs elsewhere in metabolism. Under anaerobic conditions, NADH reoxidation in S. cerevisiae is strictly dependent on reduction of sugar to glycerol. Glycerol formation is initiated by reduction of the glycolytic intermediate dihydroxyacetone phosphate (DHAP) to glycerol 3-phosphate (glycerol-3P), a reaction catalyzed by NAD⁺-dependent glycerol 3-phosphate dehydrogenase. Subsequently, the glycerol 3-phosphate formed in this reaction is hydrolysed by glycerol-3-phosphatase to yield glycerol and inorganic phosphate. Consequently, glycerol is a major by-product during anaerobic production of ethanol by S. cerevisiae, which is undesired as it reduces overall conversion of sugar to ethanol. Further, the presence of glycerol in effluents of ethanol production plants may impose costs for waste-water treatment.

WO2014/129898 describes a recombinant cell functionally heterologous nucleic acid sequences encoding for ribulose-1,5-phosphate carboxylase/oxygenase (EC 4.1.1.39; herein abbreviated as “Rubisco”), and optionally molecular chaperones for Rubisco, and phosphoribulokinase (EC 2.7.1.19; herein abbreviated as “PRK”).

WO2015/107496 describes a recombinant cell functionally heterologous nucleic acid sequences encoding for ribulose-1,5-phosphate carboxylase/oxygenase units RbcL, RbcS and RcbX, molecular chaperones for Rubisco GroEL and GroES. In the examples PRK is expressed with a tetracyclin-inducible promoter TetO7. Thereby, a process aid is needed for this promoter i.e. the additions of a compound to the propagation which adds to the cost and complexity of the process. The said compound is doxycycline, an antibiotic, which is not preferred as an additive in the ethanol fermentation process.

WO2018/228836 describes a recombinant yeast cell functionally expressing one or more heterologous nucleic acid sequences encoding for ribulose-1,5-phosphate carboxylase/oxygenase (EC4. 1.1.39; Rubisco) and optionally one or more molecular chaperones for Rubisco, and further comprising one or more phosphoribulokinase (EC2.7. 1.19; PRK), wherein one or more genes of the non-oxidative branch of the pentose phosphate pathway are overexpressed.

Although the described processes and yeast cells in WO2014/129898 and WO2018/228836 are advantageous, there is a continuing need for improvement. In an industrial environment the reduction in glycerol production by the above recombinant yeast cells can potentially affect their osmotolerance and their stress response to the external environment. Especially under challenging process conditions, for example when applying a fermentation medium having a high dry solids content and/or a high fermentation temperature, this may lead to a decline of the cell population and/or cell activity at the end of the fermentation period. It would be an advancement in the art to provide a process, and yeast cells for use therein, wherein the yeast cells have an improved robustness under high dry solids/high dry matter conditions and/or high temperatures. In addition, it would be an advancement in the art to provide yeast cells that have a reduced accumulation of glucose and/or total sugar content within the yeast cell. That is, it would be an advancement in the art to achieve a continued performance of the yeast cell and/or a low concentration of remaining glucose at the end of the fermentation, even where a high concentration of glucose is present at the start and/or throughout the fermentation.

### SUMMARY OF THE INVENTION

The inventors have now surprising found that the processes and yeast cells of WO2014/129898 and WO2018/228836 can be even further improved by promoting a transketolase with a specific promoter.

Accordingly the invention provides a recombinant yeast cell functionally expressing:
a) a heterologous nucleic acid sequence encoding a protein having ribulose-1,5-phosphate carboxylase/oxygenase activity (EC4.1.1.39; Rubisco), a heterologous nucleic acid sequence encoding a protein having phosphoribulokinase activity (EC2.7.1.19; PRK) and optionally one or more nucleic acid sequences encoding for molecular chaperones for the protein having ribulose-1,5-phosphate carboxylase/oxygenase activity; and
b) a nucleic acid sequence encoding a protein having transketolase activity (EC 2.2.1.1).
wherein the expression of the nucleic acid sequence encoding the protein having transketolase activity is under control of a promoter (the “TKL promoter”), which TKL promoter has an anaerobic/aerobic expression ratio for the transketolase of 2 or more.

In addition, the invention provides a process for the production of ethanol, comprising converting a carbon source, such as a carbohydrate or another organic carbon source, using the above recombinant yeast cell, thereby suitably forming ethanol.

Advantageously, use of the above recombinant yeast cell and/or the above process results in an improved robustness. Such is especially advantageous when a medium having a high dry solids content is applied and/or if a high fermentation temperature is applied.

A process for the production of ethanol from a carbon source, such as a carbohydrate, can advantageously be carried out in the presence of a saccharolytic enzyme, such as glucoamylase, to convert polysaccharides and/or oligosaccharides into glucose. When the process is carried out in a medium with a high dry matter content, for example after starting the process with a high concentration of corn mash, the concentration of glucose in the medium can become very high. Without wishing to be bound by any kind of theory, it is believed that a high concentration of glucose can cause osmotic stress for the yeast cell, causing the yeast cell to stop performing and even die.

Without wishing to be bound by any kind of theory it is believed that, compared to a yeast cell not comprising the TKL promoter, the above recombinant yeast cell allows for reduced accumulation of glucose and/or other sugars within the yeast cell, thereby suitably allowing for an improved robustness.

The advantages are illustrated by the examples. In the examples fermentation is carried out at a high dry matter content of 36% w/w. As illustrated by the examples the recombinant yeast cell according to the invention, and the process according to the invention, allow for a continued performance of the yeast cell and/or continued conversion of the glucose. Even in a medium comprising a concentration of glucose as high as 36% w/w and/or temperatures as high as 3200, the recombinant yeast cell is still converting carbohydrates into ethanol after 66 hours. As a result a low concentration of remaining glucose can be obtained at the end of the fermentation, even where a high concentration of glucose is present at the start and/or throughout the fermentation.

### BRIEF DESCRIPTION OF THE SEQUENCE LISTING

This application contains a Sequence Listing in computer readable form, which is incorporated herein by reference. An overview is provided by Table 1 below.

TABLE 1
Overview of sequence listings:

| SEQ ID No: | Enzyme Name | Origin | Type |
|---|---|---|---|
| SEQ ID No: 1 | Ribulose-1,5-biphosphate | Thiobacillus denitrificans | protein |
| | carboxylase oxygenase (RuBisCO) | | |
| | as previously described in | | |
| | WO2014/129898 | | |
| SEQ ID No: 2 | Ribulose-1,5-biphosphate | Artificial sequence; based on | DNA |
| | carboxylase oxygenase (RuBisCO) | cbbM gene from Thiobacillus | |
| | as previously described in | denitrificans - pBTWW002, | |
| | WO2014/129898 | codon optimized | |
| SEQ ID No: 3 | Phosphoribulokinase (PRK) as | Spinacea oleracea | protein |
| | previously described in | | |
| | WO2014/129898 | | |
| SEQ ID No: 4 | Phosphoribulokinase (PRK) as | Artificial sequence; based on | DNA |
| | previously described in | prk gene from Spinacea | |
| | WO2014/129898 | oleracea, codon optimized | |
| SEQ ID NO: 5 | (long) chaperone groEL as | Artificial sequence; Translated | protein |
| | previously described in | protein sequence based on | |
| | WO2014/129898 | GroEL of Escherichia coli | |
| SEQ ID NO: 6 | (long) chaperone groEL as | Artificial sequence; based on | DNA |
| | previously described in | GroEL gene from Escherichia | |
| | WO2014/129898 | coli, codon optimized | |
| SEQ ID NO: 7 | (short) chaperone groES as | Artificial sequence; Translated | protein |
| | previously described in | protein sequence based on | |
| | WO2014/129898 | GroES of Escherichia coli | |
| SEQ ID NO: 8 | (short) chaperone groES as | Artificial sequence; based on | DNA |
| | previously described in | GroES gene from Escherichia | |
| | WO2014/129898 | coli, codon optimized | |
| SEQ ID NO: 9 | Transketolase | Saccharomyces cerevisiae | protein |
| SEQ ID NO: 10 | Transketolase | Saccharomyces cerevisiae | DNA |
| SEQ ID NO: 11 | Transketolase (DAS1) | Komagataella phaffii | protein |
| | | CBS 7435 | |
| SEQ ID NO: 12 | Transketolase (DAS2) | Komagataella phaffii | protein |
| | | CBS 7435 | |
| SEQ ID NO: 13 | Transketolase | Candida tenuis | protein |
| SEQ ID NO: 14 | Transketolase | Debaryomyces | protein |
| SEQ ID NO: 15 | Transketolase | Kuraishia capsulata | protein |
| | | CBS 1993 | |
| SEQ ID NO: 16 | Transketolase | Candida arabinofermentans | protein |
| | | NRRL YB-2248 | |
| SEQ ID NO: 17 | Transketolase | Metschnikowia pulcherrima | protein |
| SEQ ID NO: 18 | Transketolase | Meyerozyma | protein |
| SEQ ID NO: 19 | Transketolase | Diutina rugosa | protein |
| SEQ ID NO: 20 | Transketolase | Pachysolen tannophilus | protein |
| | | NRRL Y-2460 | |
| SEQ ID NO: 21 | Transketolase | Rasamsonia emersonii | protein |
| | | CBS 393.64 | |
| SEQ ID NO: 22 | Transketolase | Kluyveromyces marxianus | protein |
| | | (strain DMKU3-1042/BCC | |
| | | 29191/NBRC 104275) | |
| SEQ ID NO: 23 | Transketolase | Ogataea parapolymorpha | protein |
| | | DL-1 | |
| SEQ ID NO: 24 | Transketolase | Artificial sequence; based on | protein |
| | | Transketolase protein from | |
| | | Komagataella phaffii | |
| SEQ ID NO: 25 | Transketolase | Komagataella phaffii | protein |
| | | (Pichia pastoris) | |
| SEQ ID NO: 26 | Transketolase | Komagataella phaffii | DNA |
| | | (Pichia pastoris) | |
| SEQ ID NO: 27 | Transketolase | Kluyveromyces lactis | protein |
| SEQ ID NO: 28 | Transketolase | Kluyveromyces lactis | DNA |
| SEQ ID NO: 29 | Promoter Motif | Artificial sequence | DNA |
| SEQ ID NO: 30 | Promoter Motif | Artificial sequence | DNA |
| SEQ ID NO: 31 | Promoter ANB1 | Saccharomyces cerevisiae | DNA |
| SEQ ID NO: 32 | Promoter DAN1 | Saccharomyces cerevisiae | DNA |
| SEQ ID NO: 33 | Glycerol dehydrogenase | Klebsiella pneumoniae | protein |
| | (Kpne_gldA) | | |
| SEQ ID NO: 34 | Glycerol dehydrogenase | Enterococcus aerogenes | protein |
| | (Eaer_gldA) | | |
| SEQ ID NO: 35 | Glycerol dehydrogenase (Ya_gldA) | Yersinia aldovae | protein |
| SEQ ID NO: 36 | Glycerol dehydrogenase (Ec_gldA) | Escherichia coli | protein |
| SEQ ID NO: 37 | Glycerol dehydrogenase (Ec_gldA) | Artificial sequence; based on | DNA |
| | | gldA gene from Escherichia | |
| | | coli - codon optimized | |
| SEQ ID NO: 38 | Dihydroxyacetone kinase | Saccharomyces cerevisiae | protein |
| | (Sc_DAK1) | | |
| SEQ ID NO: 39 | Dihydroxyacetone kinase | Saccharomyces cerevisiae | protein |
| | (Sc_DAK2) | | |
| SEQ ID NO: 40 | Dihydroxyacetone kinase | Klebsiella pneumoniae | protein |
| | (Kpne_dhaK) | | |
| SEQ ID NO: 41 | Dihydroxyacetone kinase | Yarrowia lipolytica | protein |
| | (Ylip_DAK1) | | |
| SEQ ID NO: 42 | Dihydroxyacetone kinase | Schizosaccharomyces pombe | protein |
| | (Spom_DAK1) | | |
| SEQ ID NO: 43 | Dihydroxyacetone kinase | Saccharomyces cerevisiae | DNA |
| | (Sc_DAK1) | | |
| SEQ ID NO: 44 | Dihydroxyacetone kinase | Saccharomyces cerevisiae | DNA |
| | (Sc_DAK2) | | |
| SEQ ID NO: 45 | Aquaporin 9 (Drer_T3) | Danio rerio | protein |
| SEQ ID NO: 46 | ZYRO0E01210p (Zrou_T5) | Zygosaccharomyces rouxii | protein |
| SEQ ID NO: 47 | Glucoamylase (GA) | Punctularia strigosozonata | protein |
| SEQ ID NO: 48 | Glucoamylase (GA) | Punctularia strigosozonata | protein |
| SEQ ID NO: 49 | Glucoamylase (GA) | Artificial sequence, based on | protein |
| | | Punctularia strigosozonata | |
| SEQ ID NO: 50 | “fragmentA” | Artificial Sequence | DNA |
| SEQ ID NO: 51 | “fragmentB” | Artificial Sequence | DNA |
| SEQ ID NO: 52 | “fragmentC” | Artificial Sequence | DNA |
| SEQ ID NO: 53 | “fragmentD” | Artificial Sequence | DNA |
| SEQ ID NO: 54 | “fragmentE” | Artificial Sequence | DNA |
| SEQ ID NO: 55 | repair DNA fragment | Artificial Sequence | DNA |
| SEQ ID NO: 56 | Terminator EBO1 (Sc_EBO1t) | Saccharomyces cerevisiae | DNA |
| SEQ ID NO: 57 | Promoter ENO2 (Sc_ENO2p) | Saccharomyces cerevisiae | DNA |
| SEQ ID NO: 58 | S. cerevisiae YOX1 terminator | Saccharomyces cerevisiae | DNA |
| | (Sc_YOX1t) | | |
| SEQ ID NO: 59 | INT14.02 protospacer | Artificial Sequence | DNA |
| SEQ ID NO: 60 | Sc_INT14.02_FLANK5 | Saccharomyces cerevisiae | DNA |
| SEQ ID NO: 61 | Sc_INT14.02_FLANK3 | Saccharomyces cerevisiae | DNA |
| SEQ ID NO: 62 | INT95 protospacer | Artificial Sequence | DNA |
| SEQ ID NO: 63 | Sc_INT95B_FLANK5 | Saccharomyces cerevisiae | DNA |
| SEQ ID NO: 64 | Sc_INT95B_FLANK3 | Saccharomyces cerevisiae | DNA |
| SEQ ID NO: 65 | GPD2 protospacer | Artificial Sequence | DNA |

In the context of this patent application, each of the above protein/amino acid sequences is preferably encoded by a DNA/nucleic acid sequence that is codon-pair optimized for expression in a yeast, more preferably for expression in a Saccharomyces cerevisiae yeast.

### DETAILED DESCRIPTION OF THE INVENTION

### Definitions

Unless defined otherwise or clearly indicated by context, all technical and scientific terms used herein have the same meaning as commonly understood by one of ordinary skill in the art.

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element. When referring to a noun (e.g. a compound, an additive, etc.) in the singular, the plural is meant to be included. Thus, when referring to a specific moiety, e.g. “gene”, this means “at least one” of that gene, e.g. “at least one gene”, unless specified otherwise.

When referring to a compound of which several isomers exist (e.g. a D and an L enantiomer), the compound in principle includes all enantiomers, diastereomers and cis/trans isomers of that compound that may be used in the particular aspect of the invention; in particular when referring to such as compound, it includes the natural isomer(s).

Unless explicitly indicated otherwise, the various embodiments of the invention described herein can be cross-combined.

The term “carbon source” refers to a source of carbon, preferably a compound or molecule comprising carbon. Preferably the carbon source is a carbohydrate. A carbohydrate is understood herein to be an organic compound made of carbon, oxygen and hydrogen. Suitably the carbon source may be selected from the group consisting of mono-, di- and/or polysaccharides, acids and acid salts. More preferably the carbon source is a compound selected from the group consisting of glucose, arabinose, xylose, galactose, mannose, rhamnose, fructose, glycerol, and acetic acid or a salt thereof.

The terms “dry matter” and “dry solids”, abbreviated respectively as “DM” and “DS”, are used interchangeably herein and refer to material remaining after removal of water. Dry matter content can be determined by any method known to the person skilled in the art therefore.

The term “ferment”, and variations thereof such as “fermenting”, “fermentation” and/or “fermentative”, is used herein in a classical sense, i.e. to indicate that a process is or has been carried out under anaerobic conditions. An anaerobic fermentation is herein defined to be a fermentation carried out under anaerobic conditions. Anaerobic conditions are herein defined as conditions without any oxygen or in which essentially no oxygen is consumed by the yeast cell. Conditions in which essentially no oxygen is consumed suitably corresponds to an oxygen consumption of less than 5 mmol/l·h⁻¹, in particular to an oxygen consumption of less than 2.5 mmol/l·h⁻¹, or less than 1 mmol/l·h⁻¹. More preferably 0 mmol/L/h is consumed (i.e. oxygen consumption is not detectable). This suitably corresponds to a dissolved oxygen concentration in a culture broth of less than 5% of air saturation, more suitably to a dissolved oxygen concentration of less than 1% of air saturation, or less than 0.2% of air saturation.

The term “fermentation process” refers to a process for the preparation or production of a fermentation product.

The term “cell” refers to a eukaryotic or prokaryotic organism, preferably occurring as a single cell. In the present invention the cell is a recombinant yeast cell. That is, the recombinant cell is selected from the group of genera consisting of yeast.

The terms “yeast” and “yeast cell” are used herein interchangeably and refer to a phylogenetically diverse group of single-celled fungi, most of which are in the division of Ascomycota and Basidiomycota. The budding yeasts (“true yeasts”) are classified in the order Saccharomycetales. The yeast cell according to the invention is preferably a yeast cell derived from the genus of Saccharomyces. More preferably the yeast cell is a yeast cell of the species Saccharomyces cerevisiae.

The term “recombinant”, for example referring to a “recombinant yeast”, a “recombinant cell”, “recombinant micro-organism” and/or “recombinant strain” as used herein, refers to a yeast, cell, micro-organism or strain, respectively, containing nucleic acid which is the result of one or more genetic modifications. Simply put the yeast, cell, micro-organism or strain contains a different combination of nucleic acid from (either of) its parent(s). To construe a recombinant yeast, cell, micro-organism or strain, recombinant DNA technique(s) and/or another mutagenic technique(s) can be used. For example a recombinant yeast and/or a recombinant yeast cell may comprise nucleic acid not present in the corresponding wild-type yeast and/or cell, which nucleic acid has been introduced into that yeast and/or yeast cell using recombinant DNA techniques (i.e. a transgenic yeast and/or cell), or which nucleic acid not present in said wild-type yeast and/or cell is the result of one or more mutations—for example using recombinant DNA techniques or another mutagenesis technique such as UV-irradiation—in a nucleic acid sequence present in said wild-type yeast and/or yeast cell (such as a gene encoding a wild-type polypeptide) or wherein the nucleic acid sequence of a gene has been modified to target the polypeptide product (encoding it) towards another cellular compartment. Further, the term “recombinant” may suitably relate to a yeast, cell, micro-organism or strain from which nucleic acid sequences have been removed, for example using recombinant DNA techniques.

By a recombinant yeast comprising or having a certain activity is herein understood that the recombinant yeast may comprise one or more nucleic acid sequences encoding for a protein having such activity. Hence allowing the recombinant yeast to functionally express such a protein or enzyme.

The term “functionally expressing” means that there is a functioning transcription of the relevant nucleic acid sequence, allowing the nucleic acid sequence to actually be transcribed, for example resulting in the synthesis of a protein.

The term “transgenic” as used herein, for example referring to a “transgenic yeast” and/or a “transgenic cell”, refers to a yeast and/or cell, respectively, containing nucleic acid not naturally occurring in that yeast and/or cell and which has been introduced into that yeast and/or cell using for example recombinant DNA techniques, such as a recombinant yeast and/or cell.

The term “mutated” as used herein regarding proteins or polypeptides means that, as compared to the wild-type or naturally occurring protein or polypeptide sequence, at least one amino acid has been replaced with a different amino acid, inserted into, or deleted from the amino acid sequence. The replacement, insertion or deletion of the amino acid can for example be achieved via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989), published by Cold Spring Harbor Publishing).

The term “mutated” as used herein regarding genes means that, as compared to the wild-type or naturally occurring nucleic acid sequence, at least one nucleotide in the nucleic acid sequence of a gene or a regulatory sequence thereof, has been replaced with a different nucleotide, inserted into, or deleted from the nucleic acid sequence. The replacement, insertion or deletion of the amino acid can for example be achieved via mutagenesis, resulting for example in the transcription of a protein sequence with a qualitatively of quantitatively altered function or the knock-out of that gene. In the context of this invention an “altered gene” has the same meaning as a mutated gene.

The term “gen” or “gene”, as used herein, refers to a nucleic acid sequence that can be transcribed into mRNAs that are then translated into protein. A gene encoding for a certain protein refers to the one or more nucleic acid sequence(s) encoding for such a protein.

The term “nucleic acid” or “nucleotide” as used herein, refers to a monomer unit in a deoxyribonucleotide or ribonucleotide polymer, i.e. a polynucleotide, in either single or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). For example, a certain enzyme that is defined by a nucleotide sequence encoding the enzyme, includes (unless otherwise limited) the nucleotide sequence hybridising to the reference nucleotide sequence encoding the enzyme. A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “nucleotide sequence” and “nucleic acid sequence” are used interchangeably herein. An example of a nucleic acid sequence is a DNA sequence.

The terms “polypeptide”, “peptide” and “protein” are used interchangeably herein to refer to a polymer of amino acid residues, for example illustrated by an amino acid sequence. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation.

The term “enzyme” refers herein to a protein having a catalytic function. Where a protein catalyzes a certain biological reaction, the terms “protein” and “enzyme” may be used interchangeable herein. When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via www.ncbi.nlm.nih.gov/, (as available on 1 Oct. 2020) unless specified otherwise.

Every nucleic acid sequence herein that encodes a polypeptide also includes any conservatively modified variants thereof. This includes that, by reference to the genetic code, it describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

The term “functional homologue” (or in short “homologue”) of a polypeptide and/or amino acid sequence having a specific sequence (e.g. “SEQ ID NO: X”), as used herein, refers to a polypeptide and/or amino acid sequence comprising said specific sequence with the proviso that one or more amino acids are mutated, substituted, deleted, added, and/or inserted, and which polypeptide has (qualitatively) the same enzymatic functionality for substrate conversion.

The term “functional homologue” (or in short “homologue”) of a polynucleotide and/or nucleic acid sequence having a specific sequence (e.g. “SEQ ID NO: X”), as used herein, refers to a polynucleotide and/or nucleic acid sequence comprising said specific sequence with the proviso that one or more nucleic acids are mutated, substituted, deleted, added, and/or inserted, and which polynucleotide encodes for a polypeptide sequence that has (qualitatively) the same enzymatic functionality for substrate conversion. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. A comparison of sequences and determination of percent identity between two sequences can be accomplished using a mathematical algorithm. The skilled person will be aware of the fact that several different computer programs are available to align two sequences and determine the homology between two sequences (Kruskal et al., “An overview of sequence comparison: Time warps, string edits, and macromolecules”, (1983), Society for Industrial and Applied Mathematics (SIAM), Vol 25, No. 2, pages 201-237 and D. and the handbook edited by Sankoff and J. B. Kruskal, (ed.), “Time warps, string edits and macromolecules: the theory and practice of sequence comparison”, (1983), pp. 1-44, published by Addison-Wesley Publishing Company, Massachusetts USA).

The percent identity between two amino acid sequences can be determined using the Needleman and Wunsch algorithm for the alignment of two sequences. (Needleman et al “A General Method Applicable to the Search for Similarities in the Amino Acid Sequence of Two Proteins” (1970) J. Mol. Biol. Vol. 48, pages 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package is used (version 2.8.0 or higher, see Rice et al, “EMBOSS: The European Molecular Biology Open Software Suite” (2000), Trends in Genetics vol. 16, (6) pages 276-277, http://emboss.bioinformatics.nl/). For protein sequences, EBLOSUM62 is used for the substitution matrix. For nucleotide sequences, EDNAFULL is used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labelled in the output of the program as “longest-identity”.

A variant of a nucleotide or amino acid sequence disclosed herein may also be defined as a nucleotide or amino acid sequence having one or more mutations, substitutions, insertions and/or deletions as compared to the nucleotide or amino acid sequence specifically disclosed herein (e.g. in de the sequence listing).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. In an embodiment, conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and, Val to lie or Leu.

Nucleotide sequences of the invention may also be defined by their capability to hybridise with parts of specific nucleotide sequences disclosed herein, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

“Expression” refers to the transcription of a gene into structural RNA (rRNA, tRNA) or messenger RNA (mRNA) with subsequent translation into a protein.

“Overexpression” refers to expression of a gene, respectively a nucleic acid sequence, by a recombinant cell in excess to its expression in a corresponding wild-type cell. Such overexpression can for example be arranged for by: increasing the frequency of transcription of one or more nucleic acid sequences, for example by operational linking of the nucleic acid sequence to a promoter functional within the recombinant cell; and/or by increasing the number of copies of a certain nucleic acid sequence.

The terms “upregulate”, “upregulated” and “upregulation” refer to a process by which a cell increases the quantity of a cellular component, such as RNA or protein. Such an upregulation may be in response to or caused by a genetic modification.

By the term “pathway” or “metabolic pathway” is herein understood a series of chemical reactions in a cell that build and breakdown molecules.

Nucleic acid sequences (i.e. polynucleotides) or proteins (i.e. polypeptides) may be native or heterologous to the genome of the host cell.

“Native”, “homologous” or “endogenous” with respect to a host cell, means that the nucleic acid sequence does naturally occur in the genome of the host cell or that the protein is naturally produced by that cell. The terms “native”, “homologous” and “endogenous” are used interchangeable herein.

As used herein, “heterologous” may refer to a nucleic acid sequence or a protein. For example, “heterologous”, with respect to the host cell, may refer to a polynucleotide that does not naturally occur in that way in the genome of the host cell or that a polypeptide or protein is not naturally produced in that manner by that cell. A heterologous nucleic acid sequence is a nucleic acid that originates from a foreign species, or, if from the same species, is substantially modified from its native form in composition and/or genomic locus by deliberate human intervention. For example, a promoter operably linked to a native structural gene is from a species different from that from which the structural gene is derived, or, if from the same species, one or both are substantially modified from their original form. A heterologous protein may originate from a foreign species or, if from the same species, is substantially modified from its original form by deliberate human intervention. That is, heterologous protein expression involves expression of a protein that is not naturally expressed in that way in the host cell. The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding a certain protein or enzyme with a specific activity can be expressed in such a eukaryotic system. In some embodiments, transformed/transfected cells may be employed as expression systems for the expression of the enzymes. Expression of heterologous proteins in yeast is well known. Sherman, F., et al., Methods in Yeast Genetics, (1986), published by Cold Spring Harbor Laboratory, is a well-recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene or other (part of) nucleic acid sequence. Suitably, a promoter is located in the 5′-region of a gene, proximal to the transcriptional start site of a (structural) gene. Promoter sequences may be constitutive, inducible or repressible. In an embodiment there is no (external) inducer needed.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleic acid sequence that comprises in the 5′ to 3′ direction and operably linked: (a) a yeast-recognized transcription and translation initiation region, (b) a coding sequence for a polypeptide of interest, and (c) a yeast-recognized transcription and translation termination region.

“Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e. operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

By “host cell” is herein understood a cell, such as a yeast cell, that is to be transformed with one or more nucleic acid sequences encoding for one or more heterologous proteins, to construe a transformed cell, also referred to as a recombinant cell. For example, the transformed cell may contain a vector and may support the replication and/or expression of the vector.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome. “Transformation” and “transforming”, as used herein, refers to the insertion of an exogenous polynucleotide (i.e. an exogenous nucleic acid sequence) into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

By “constitutive expression” and “constitutively expressing” is herein understood that there is a continuous transcription of a nucleic acid sequence. That is, the nucleic acid sequence is transcribed in an ongoing manner. Constitutively expressed genes are always “on”.

By “anaerobic constitutive expression” is herein understood that nucleic acid sequence is constitutively expressed in an organism under anaerobic conditions. That is, under anaerobic conditions the nucleic acid sequence is transcribed in an ongoing manner, i.e. under such anaerobic conditions the genes are always “on”.

By “disruption” is herein understood any disruption of activity, including, but not limited to, deletion, mutation and reduction of the affinity of the disrupted gene and expression of RNA complementary to such disrupted gene. It includes all nucleic acid modifications such as nucleotide deletions or substitutions, gene knock-outs, and other actions which affect the translation or transcription of the corresponding polypeptide and/or which affect the enzymatic (specific) activity, its substrate specificity, and/or or stability. It also includes modifications that may be targeted on the coding sequence or on the promotor of the gene. A gene disruptant is a cell that has one or more disruptions of the respective gene. Native to yeast herein is understood as that the gene is present in the yeast cell before the disruption.

The term “encoding” has the same meaning as “coding for”. Thus, by way of example, “one or more genes encoding a transketolase” has the same meaning as “one or more genes coding for a transketolase”.

As far as genes or nucleic acid sequences encoding a protein or an enzyme are concerned, the phrase “one or more nucleic acid sequences encoding a X”, wherein X denotes a protein, has the same meaning as “one or more nucleic acid sequences encoding a protein having X activity”. Thus, by way of example, “one or more nucleic acid sequences encoding a transketolase” has the same meaning as “one or more nucleic acid sequences encoding a protein having transketolase activity”.

The abbreviation “NADH” refers to reduced, hydrogenated form of nicotinamide adenine dinucleotide. The abbreviation “NAD+” refers to the oxidized form of nicotinamide adenine dinucleotide. Nicotinamide adenine dinucleotide may act as a so-called cofactor, assisting in biochemical reactions and/or transformations in a cell.

“NADH dependent” or “NAD+ dependent” is herein equivalent to NADH specific and “NADH dependency” or “NAD+ dependency” is herein equivalent to NADH specificity.

By a “NADH dependent” or “NAD+ dependent” enzyme is herein understood an enzyme that is exclusively depended on NADH/NAD+ as a co-factor or that is predominantly dependent on NADH/NAD+ as a cofactor, i.e. as contrasted to other types of co-factor. By an “exclusive NADH/NAD+ dependent” enzyme is herein understood an enzyme that has an absolute requirement for NADH/NAD+ over NADPH/NADP+. That is, it is only active when NADH/NAD+ is applied as cofactor. By a “predominantly NADH/NDA+-dependent” enzyme is herein understood an enzyme that has a higher specificity and/or a higher catalytic efficiency for NADH/NAD+ as a cofactor than for NADPH/NADP+ as a cofactor.

The enzyme's specificity characteristics can be described by the formula:

$$1 < {K}_{m} {NADP}^{+} / {K}_{m} {NAD}^{+} {<}^{∞} ( infinity )$$

wherein Kₘ is the so-called Michaelis constant.

For a predominantly NADH-dependent enzyme, preferably KₘNADP⁺/KₘNAD⁺ is between 1 and 1000, between 1 and 500, between 1 and 200, between 1 and 100, between 1 and 50, between 1 and 10, between 5 and 100, between 5 and 50, between 5 and 20 or between 5 and 10.

The Kₘ's for the enzymes herein can be determined as enzyme specific, for NAD⁺ and NADP⁺ respectively, using know analysis techniques, calculations and protocols. These are described for instance in Lodish et al., Molecular Cell Biology 6^{th} Edition, Ed. Freeman, pages 80 and 81, e.g. FIG. 3-22. For an predominantly NADH-dependent enzyme, preferably the ratio of the catalytic efficiency for NADPH/NADP+ as a cofactor (k_{cat}/Kₘ)^{NADP+} to NADH/NAD+ as cofactor (k_{cat}/Kₘ)^{NAD+}, i.e. the catalytic efficiency ratio (k_{cat}/Kₘ)^{NADP+}: (k_{cat}/Kₘ)^{NAD+}, is more than 1:1, more preferably equal to or more than 2:1, still more preferably equal to or more than 5:1, even more preferably equal to or more than 10:1, yet even more preferably equal to or more than 20:1, even still more preferably equal to or more than 100:1, and most preferably equal to or more than 1000:1. There is no upper limit, but for practical reasons the predominantly NADH-dependent enzyme may have a catalytic efficiency ratio (k_{cat}/Kₘ)^{NADP+}:(k_{cat}/Kₘ)^{NAD+} of equal to or less than 1.000.000.000:1 (i.e. 1.109:1).

### The Yeast Cell

The recombinant yeast cell is preferably a yeast cell, or derived from, a host yeast cell, from the genus of Saccharomycetaceae or the genus of Schizosaccharomycetaceae. That is, preferably the host cell from which the recombinant yeast cell is derived is a yeast cell from the genus of Saccharomycetaceae or the genus of Schizosaccharomycetaceae.

Examples of suitable yeast cells include Saccharomyces, such as Saccharomyces cerevisiae, Saccharomyces eubayanus, Saccharomyces jurei, Saccharomyces pastorianus, Saccharomyces beticus, Saccharomyces fermentati, Saccharomyces paradoxus, Saccharomyces uvarum and Saccharomyces bayanus.

Examples of suitable yeast cells further include Schizosaccharomyces, such as Schizosaccharomyces pombe, Schizosaccharomyces japonicus, Schizosaccharomyces octosporus and Schizosaccharomyces cryophilus.

Other exemplary yeasts include Torulaspora such as Torulaspora delbrueckii; Kluyveromyces such as Kluyveromyces marxianus; Pichia such as Pichia stipitis, Pichia pastoris or pichia angusta; Zygosaccharomyces such as Zygosaccharomyces bailii; Brettanomyces such as Brettanomyces inter medius; Brettanomyces bruxellensis, Brettanomyces anomalus, Brettanomyces custersianus, Brettanomyces naardenensis, Brettanomyces nanus, Dekkera bruxellensis and Dekkera anomala; Metschmkowia, Issatchenkia, such as Issatchenkia orientalis, Kloeckera such as Kloeckera apiculata; and Aureobasidium such as Aureobasidium pullulans.

The yeast cell is preferably a yeast cell of the genus Schizosaccharomyces, herein also referred to as a Schizosaccharomyces yeast cell, or a yeast cell of the genus Saccharomyces, herein also referred to as a Saccharomyces yeast cell. More preferably the yeast cell is a yeast cell derived from a yeast cell of the species Saccharomyces cerevisiae, herein also referred to as a Saccharomyces cerevisae yeast cell. That is, preferably the host cell from which the recombinant yeast cell is derived is a yeast cell from the species Saccharomyces cerevisiae.

Preferably the yeast cell is an industrial yeast cell. The living environments of yeast cells in industrial processes are significantly different from that in the laboratory. Industrial yeast cells must be able to perform well under multiple environmental conditions which may vary during the process. Such variations include changes in nutrient sources, pH, ethanol concentration, temperature, oxygen concentration, etc., which together have potential impact on the cellular growth and ethanol production of the yeast cell. An industrial yeast cell can be understood to refer to a yeast cell that, when compared to a laboratory counterpart, has a more robust performance. That is, when compared to a laboratory counterpart, the industrial yeast cell shows less variation in performance when one or more environmental conditions selected from the group of nutrient sources, pH, ethanol concentration, temperature, oxygen concentration, are varied during fermentation.

Preferably, the yeast cell is constructed on the basis of an industrial yeast cell as a host, wherein the construction is conducted as described hereinafter. Examples of industrial yeast cells are Ethanol Red® (Fermentis) Fermiol® (DSM) and Thermosacc® (Lallemand).

The recombinant yeast cell described herein may be derived from any host cell capable of producing a fermentation product. Preferably the host cell is a yeast cell, more preferably an industrial yeast cell as described herein above. Preferably the yeast cell described herein is derived from a host cell having the ability to produce ethanol.

The yeast cell described herein may be derived from the host cell through any technique known by one skilled in the art to be suitable therefore. Such techniques may include any one or more of mutagenesis, recombinant DNA technology (including, but not limited to, CRISPR-CAS techniques), selective and/or adaptive evolution, mating, cell fusion, and/or cytoduction between yeast strains. Suitably the one or more desired genes are incorporated in the yeast cell by a combination of one or more of the above techniques.

The recombinant yeast cells according to the invention are preferably inhibitor tolerant, i.e. they can withstand common inhibitors at the level that they typically have with common pretreatment and hydrolysis conditions, so that the recombinant yeast cells can find broad application, i.e. it has high applicability for different feedstock, different pretreatment methods and different hydrolysis conditions. In an embodiment the recombinant yeast cell is inhibitor tolerant. Inhibitor tolerance is resistance to inhibiting compounds. The presence and level of inhibitory compounds in lignocellulose may vary widely with variation of feedstock, pretreatment method hydrolysis process. Examples of categories of inhibitors are carboxylic acids, furans and/or phenolic compounds. Examples of carboxylic acids are lactic acid, acetic acid or formic acid. Examples of furans are furfural and hydroxy-methylfurfural. Examples or phenolic compounds are vanillin, syringic acid, ferulic acid and coumaric acid. The typical amounts of inhibitors are for carboxylic acids: several grams per liter, up to 20 grams per liter or more, depending on the feedstock, the pretreatment and the hydrolysis conditions. For furans: several hundreds of milligrams per liter up to several grams per liter, depending on the feedstock, the pretreatment and the hydrolysis conditions. For phenolics: several tens of milligrams per liter, up to a gram per liter, depending on the feedstock, the pretreatment and the hydrolysis conditions.

In an embodiment, the recombinant yeast cell is a cell that is naturally capable of alcoholic fermentation, preferably, anaerobic alcoholic fermentation. A recombinant yeast cell preferably has a high tolerance to ethanol, a high tolerance to low pH (i.e. capable of growth at a pH lower than about 5, about 4, about 3, or about 2.5) and towards organic and/or a high tolerance to elevated temperatures.

### Transketolase

The recombinant yeast cell is suitably functionally expressing one or more nucleic acid sequence encoding for a protein having transketolase activity (EC 2.2.1.1), wherein suitably the expression of the nucleic acid sequence encoding the protein having transketolase activity is under control of a promoter (the “TKL promoter”), which TKL promoter has an anaerobic/aerobic expression ratio for the transketolase of 2 or more. Herewith is suitably meant that the expression of the transketolase (“TKL”) is at least a factor 2 higher under anaerobic conditions than under aerobic conditions. The above can alternatively be phrased as the recombinant yeast cell functionally expressing one or more nucleic acid sequences encoding for a protein having transketolase activity (or simply phrased the “transketolase” or “TKL”), wherein the transketolase is under control of a promoter (the “TKL promoter”) which has a TKL expression ratio anaerobic/aerobic of 2 or more.

A protein having transketolase activity is herein also referred to as “transketolase protein”, “transketolase enzyme” or simply “transketolase”. The “transketolase” is herein abbreviated as “TKL”.

Transketolase is an enzyme that is active within the pentose phosphate pathway of a yeast cell. The genes encoding for this pentose phosphate pathway are herein also referred to as the “PPP” genes. Preferably references in this specification to the pentose phosphate pathway are to be understood as references to the non-oxidative part of the pentose phosphate pathway. The enzymes active within the pentose phosphate pathway include the enzymes ribulose-5-phosphate isomerase (RKI), ribulose-5-phosphate epimerase (RPE), transketolase (TKL) and transaldolase (TAL).

The enzyme “transketolase” (EC 2.2.1.1) is herein defined as an enzyme that catalyses the reaction: D-ribose 5-phosphate+D-xylulose 5-phosphate<->sedoheptulose 7-phosphate+D-glyceraldehyde 3-phosphate and vice versa.

The enzyme is also known as glycolaldehydetransferase or sedoheptulose-7-phosphate:D-glyceraldehyde-3-phosphate glycolaldehydetransferase. A certain transketolase can be further defined by its amino acid sequence. Likewise a transketolase can be further defined by a nucleotide sequence encoding the transketolase. As explained in detail above under definitions, a certain transketolase that is defined by a nucleotide sequence encoding the enzyme, includes (unless otherwise limited) the nucleotide sequence hybridising to such nucleotide sequence encoding the transketolase.

Native yeasts may comprise one or two transketolase genes. In addition to a first transketolase gene “TKL1”, some yeasts, such as for example Saccharomyces cerevisiae, comprises the paralog “TKL2”, a second transketolase gene.

Suitably the recombinant yeast cells according to the invention may comprise a TKL1 gene and/or a TKL2 gene.

That is, suitably the recombinant yeast cell may comprise:
a nucleic acid sequence encoding for TKL1 (e.g. a gene “TKL1”); or
a nucleic acid sequence encoding for TKL2 (e.g. a gene “TKL2”); or
both a nucleic acid sequence encoding for TKL1 (e.g. a gene “TKL1”) and a nucleic acid sequence encoding for TKL2 (e.g. a gene “TKL2”).

Preferably the recombinant yeast cell comprises a nucleotide sequence encoding for transketolase TKL1. That is, preferably the recombinant yeast cell comprises a TKL1 gene.

The recombinant yeast cell may comprise one or more copies, suitably in the range from equal to or more than 1 to equal to or less than 30 copies, preferably in the range equal to or more than 1 to equal to or less than 20 copies, of a gene encoding a transketolase. More preferably the recombinant yeast cell comprises one, two, three, four, five, six, seven, eight, nine, ten, eleven or twelve copies of a gene encoding a transketolase.

The genes encoding the transketolase can be homologous genes, heterologous genes or a mixture of homologous and heterologous genes.

The recombinant yeast cell can be a recombinant yeast cell, wherein a native nucleic acid sequence encoding for a protein having transketolase activity is under control of the TKL promoter.

The recombinant yeast cell can also functionally express a heterologous nucleic acid sequence encoding a protein having transketolase activity. The protein having transketolase activity can thus be a heterologous protein having transketolase activity, i.e. a “heterologous transketolase”. A heterologous nucleic acid sequence encoding for the protein having transketolase activity, respectively a heterologous transketolase, can be present as a replacement of or in addition to a native nucleic acid sequence encoding for the protein having transketolase activity, respectively a native transketolase.

When the recombinant yeast cell comprises a heterologous nucleic acid sequence encoding for the protein having transketolase activity, respectively a heterologous transketolase, one or more native nucleic acid sequence(s) encoding for a protein having transketolase activity can be disrupted or deleted.

Alternatively, the recombinant yeast cell may comprise the heterologous nucleic acid sequence encoding for a transketolase in addition to a native nucleic acid sequence encoding for a transketolase. The recombinant yeast cell thus may or may not comprise a heterologous nucleic acid sequence encoding for the protein having transketolase activity, respectively a heterologous transketolase, in addition to a native nucleic acid sequence encoding for a protein having transketolase activity, respectively in addition to a native transketolase.

If the recombinant yeast cell comprises a heterologous nucleic acid sequence encoding for a transketolase, such heterologous nucleic acid sequence encoding for the transketolase is preferably under control of the TKL promoter.

Preferably the recombinant yeast cell comprises at least one heterologous nucleic acid sequence encoding for a transketolase, respectively at least one heterologous transketolase.

Preferably a heterologous transketolase comprises or consists of
the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27; or
a functional homologue of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27 comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27; or
a functional homologue of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27, comprising an amino acid sequence having one or more mutations, substitutions, insertions and/or deletions when compared with SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27.

More preferably the amino acid sequence of any such functional homologue has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions as compared to such amino acid sequences.

Preferably the recombinant yeast cell comprises:
one or more nucleic acid sequences encoding for one or more amino acid sequence(s) chosen from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 27; and/or
functional homologues thereof comprising a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with any of those; and/or
functional homologues thereof comprising a nucleic acid sequence having one or more mutations, substitutions, insertions and/or deletions when compared therewith.

More preferably the nucleic acid sequence of any such functional homologues has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions as compared to such nucleic acid sequences.

More preferably a heterologous transketolase is derived from a Komagataella phaffii, a yeast species also referred to as “Pichia pastoris”, such as for example the polypeptides illustrated by SEQ ID NO: 11, SEQ ID NO: 12, SEQ ID NO: 24, SEQ ID NO: 25 and functional homologues thereof comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with a polypeptides illustrated by SEQ ID NO: 11, SEQ ID NO: 12, SEQ ID NO: 24 or SEQ ID NO: 25.

Host cells from the species Saccharomyces cerevisiae are preferred. The amino acid sequence of native transketolase 1 of Saccharomyces cerevisiae is illustrated by SEQ ID NO: 9. The native nucleic acid sequence encoding transketolase 1 in Saccharomyces cerevisiae is illustrated by SEQ ID NO: 10. If a native nucleic acid sequence encoding for a protein having transketolase activity is under control of the TKL promoter, such native nucleic acid sequence preferably comprises or consists of the nucleic acid sequence of SEQ ID NO: 10 or a functional homologue thereof comprising a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 10. In analogy, if a native nucleic acid sequence encoding for a protein having transketolase activity is under control of the TKL promoter, such protein having transketolase activity preferably comprises or consists of the amino acid sequence of SEQ ID NO: 9 or a functional homologue thereof comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 9

Examples of suitable transketolases thus include:
the transketolases having an amino acid sequence of SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ SEQ ID NO: 24, SEQ ID NO: 25 and SEQ ID NO: 27; and
functional homologues thereof comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of respectively SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and/or SEQ ID NO: 27; and
functional homologues thereof comprising an amino acid sequence having one or more mutations, substitutions, insertions and/or deletions as compared to the amino acid sequence of respectively SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and/or SEQ ID NO: 27.
   More preferably the amino acid sequence of any such functional homologues has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions as compared to the amino acid sequence of respectively SEQ ID NO: 9, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and/or SEQ ID NO: 27.

In order to allow for a good expression of any heterologous transketolase in the host cell, it can be advantageous to use a heterologous transketolase that may have an amino acid sequence having equal to or more than 30%, equal to or more than 35%, equal to or more than 40%, equal to or more than 45%, equal to or more than 50%, equal to or more than 55%, equal to or more than 60%, equal to or more than 65%, equal to or more than 70%, equal to or more than 75%, equal to or more than 80%, equal to or more than 85%, equal to or more than 90% equal to or more than 95%, equal to or more than 98% or equal to or more than 99% sequence identity with the amino acid sequence of the native transketolase of the host cell.

However, it may also be preferred for the heterologous transketolase to be a heterologous transketolase that is not regulated by native (i.e. endogenous) regulators of the host cell. That is, preferably the heterologous transketolase is a transketolase enzyme of which the activity cannot be increased or decreased by molecules that are natively produced by the host cell. In order to avoid native regulators, it can be advantageous to use a heterologous transketolase in the host cell that may have an amino acid sequence having equal to or less than 99%, equal to or less than 98%, equal to or less than 95%, equal to or less than 90%, equal to or less than 85%, equal to or less than 80%, equal to or less than 75%, equal to or less than 70%, or equal to or less than 65% sequence identity with the amino acid sequence of the native transketolase of the host cell.

Therefore, more preferably a heterologous transketolase has an amino acid sequence having a percentage identity with the amino acid sequence of the native transketolase of the host cell in the range of equal to or more than 30% to equal to or less than 80%, more preferably in the range of equal to or more than 35% to equal to or less than 75%, and most preferably in the range of equal to or more than 35% to equal to or less than 70% or even equal to or less than 65%. That is, more preferably any heterologous nucleic acid sequence encoding for the protein having transketolase activity is a heterologous nucleic acid sequence encoding for a protein having transketolase activity which has an amino acid sequence having a percentage identity with the amino acid sequence of the native transketolase of the host cell in the range of equal to or more than 30% to equal to or less than 80%, more preferably in the range of equal to or more than 35% to equal to or less than 75%, and most preferably in the range of equal to or more than 35% to equal to or less than 70% or even equal to or less than 65%.

Host cells from the species Saccharomyces cerevisiae are preferred. As indicated above, the amino acid sequence of native transketolase 1 of Saccharomyces cerevisiae is illustrated by SEQ ID NO: 9, the native nucleic acid sequence encoding transketolase 1 in Saccharomyces cerevisiae is illustrated by SEQ ID NO: 10.

The recombinant yeast cell can therefore also be a recombinant Saccharomyces cerevisiae yeast cell, functionally expressing a heterologous nucleic acid sequence encoding a protein having transketolase activity, wherein:
the protein having transketolase activity comprises or consists of an amino acid sequence having in the range of equal to or more than 30% to equal to or less than 80%, more preferably in the range of equal to or more than 35% to equal to or less than 75%, and most preferably in the range of equal to or more than 35% to equal to or less than 70% or even equal to or less than 65%, sequence identity with the amino acid sequence of SEQ ID NO: 9; and/or
the heterologous nucleic acid sequence comprises or consists of a nucleic acid sequence having in the range of equal to or more than 30% to equal to or less than 80%, more preferably in the range of equal to or more than 35% to equal to or less than 75%, and most preferably in the range of equal to or more than 35% to equal to or less than 70% or even equal to or less than 65%, sequence identity with the nucleic acid sequence of SEQ ID NO: 10.

The recombinant yeast cell is therefore most preferably a recombinant Saccharomyces cerevisiae yeast cell, functionally expressing a heterologous nucleic acid sequence encoding a protein having transketolase activity, wherein:

The recombinant yeast cell may comprise one, two, or more copies of a heterologous nucleic acid sequence (e.g. a heterologous gene) encoding for a heterologous transketolase and/or one, two, or more copies of a native nucleic acid sequence (e.g. a native gene) encoding for a native transketolase. Most preferably the recombinant yeast cell may comprise one, two, three, four, five, six, seven, eight, nine, ten, eleven or twelve copies of a heterologous nucleic acid sequence (e.g. a heterologous gene) encoding for a heterologous transketolase and/or one, two, three, four, five, six, seven, eight, nine, ten, eleven or twelve copies of a native nucleic acid sequence (e.g. a native gene) encoding for a native transketolase. Most preferably the recombinant yeast cell comprises at least one heterologous gene encoding for a heterologous transketolase in addition to at least one native gene encoding for a transketolase that is native to the host cell.

Preferably the recombinant yeast cell is therefore a recombinant yeast cell comprising one, two or more copies of:
a nucleic acid sequence encoding for any of the above mentioned transketolases; and/or
a nucleic acid sequence of SEQ ID NO: 10 and/or SEQ ID NO: 26 and/or SEQ ID NO: 28; and/or
a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of respectively SEQ ID NO: 10 and/or SEQ ID NO: 26 and/or SEQ ID NO: 28; and/or
a nucleic acid sequence having one or more mutations, substitutions, insertions and/or deletions as compared to the nucleic acid sequence of respectively SEQ ID NO: 10 and/or SEQ ID NO: 26 and/or SEQ ID NO: 28, wherein more preferably this nucleic acid sequence has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions as compared to the nucleic acid sequence of respectively SEQ ID NO: 10 and/or SEQ ID NO: 26 and/or SEQ ID NO: 28.

### Optional Overexpression of One or More Other Enzymes of the PPP Pathway

The recombinant yeast cell may further optionally comprise one or more genetic modifications in the other PPP-genes, i.e. RKI, RPE and TAL, that increase the flux of the pentose phosphate pathway. Advantageously, such genetic modification(s) may lead to a further increased flux through the non-oxidative part of the pentose phosphate pathway.

The recombinant yeast cell may thus optionally comprise one or more additional genetic modifications to overexpress one or more other enzymes of the (non-oxidative part of) the pentose phosphate pathway. For example, the recombinant yeast cell may comprise one or more nucleic acid sequences to overexpress one or more of the enzymes selected from the group consisting of ribulose-5-phosphate isomerase, ribulose-5-phosphate epimerase and transaldolase.

The enzyme “ribulose 5-phosphate epimerase” (EC 5.1.3.1) is herein defined as an enzyme that catalyses the epimerisation of D-xylulose 5-phosphate into D-ribulose 5-phosphate and vice versa. The enzyme is also known as phosphoribulose epimerase; erythrose-4-phosphate isomerase; phosphoketopentose 3-epimerase; xylulose phosphate 3-epimerase; phosphoketopentose epimerase; ribulose 5-phosphate 3-epimerase; D-ribulose phosphate-3-epimerase; D-ribulose 5-phosphate epimerase; D-ribulose-5-P 3-epimerase; D-xylulose-5-phosphate 3-epimerase; pentose-5-phosphate 3-epimerase; or D-ribulose-5-phosphate 3-epimerase. A ribulose 5-phosphate epimerase may be further defined by its amino acid sequence. Likewise a ribulose 5-phosphate epimerase may be defined by a nucleotide sequence encoding the enzyme as well as by a nucleotide sequence hybridising to a reference nucleotide sequence encoding a ribulose 5-phosphate epimerase. The nucleotide sequence encoding for ribulose 5-phosphate epimerase is herein designated as RPE or RPE1.

The enzyme “ribulose 5-phosphate isomerase” (EC 5.3.1.6) is herein defined as an enzyme that catalyses direct isomerisation of D-ribose 5-phosphate into D-ribulose 5-phosphate and vice versa. The enzyme is also known as phosphopentosisomerase; phosphoriboisomerase; ribose phosphate isomerase; 5-phosphoribose isomerase; D-ribose 5-phosphate isomerase; D-ribose-5-phosphate ketol-isomerase; or D-ribose-5-phosphate aldose-ketose-isomerase. A ribulose 5-phosphate isomerase may be further defined by its amino acid sequence. Likewise a ribulose 5-phosphate isomerase may be defined by a nucleotide sequence encoding the enzyme as well as by a nucleotide sequence hybridising to a reference nucleotide sequence encoding a ribulose 5-phosphate isomerase. The nucleotide sequence encoding for ribulose 5-phosphate isomerase is herein designated RKI or RKI1.

The enzyme “transaldolase” (EC 2.2.1.2) is herein defined as an enzyme that catalyses the reaction: sedoheptulose 7-phosphate+D-glyceraldehyde 3-phosphate<->D-erythrose 4-phosphate+D-fructose 6-phosphate and vice versa. The enzyme is also known as dihydroxyacetonetransferase; dihydroxyacetone synthase; formaldehyde transketolase; or sedoheptulose-7-phosphate:D-glyceraldehyde-3-phosphate glyceronetransferase. A transaldolase may be further defined by its amino acid sequence. Likewise a transaldolase may be defined by a nucleotide sequence encoding the enzyme as well as by a nucleotide sequence hybridising to a reference nucleotide sequence encoding a transaldolase. The nucleotide sequence encoding for transketolase from is herein designated TAL or TAL1.

### TKL Promoter

The recombinant yeast cell is suitably functionally expressing one or more nucleic acid sequence encoding for a protein having transketolase activity (EC 2.2.1.1), wherein suitably the expression of the nucleic acid sequence encoding the protein having transketolase activity is under control of a promoter (the “TKL promoter”), which TKL promoter has an anaerobic/aerobic expression ratio for the transketolase of 2 or more. Herewith is suitably meant that the expression of the transketolase (“TKL”) is at least a factor 2 higher under anaerobic conditions than under aerobic conditions. The above can alternatively be phrased as the recombinant yeast cell functionally expressing one or more nucleic acid sequences encoding for a protein having transketolase activity (or simply phrased the “transketolase” or “TKL”), wherein the transketolase is under control of a promoter (the “TKL promoter”) which has a TKL expression ratio anaerobic/aerobic of 2 or more.

The TKL promoter can suitably be operably linked to the nucleic acid sequence encoding the protein having transketolase activity. Preferably, the TKL promoter is located in the 5-region of a TKL gene, more preferably it is located proximal to the transcriptional start site of a TKL gene. As indicated above, the TKL gene is preferably a TKL1 or a TKL2 gene.

Preferably the TKL promoter is ROX1 repressed. ROX1 is herein Heme-dependent repressor of hypoxic gene(s); that mediates aerobic transcriptional repression of hypoxia induced genes such as COX5b and CYC7; the repressor function is regulated through decreased promoter occupancy in response to oxidative stress; and contains an HMG domain that is responsible for DNA bending activity; involved in the hyperosmotic stress resistance. ROX1 is regulated by oxygen.

Without wishing to be limited by any kind of theory it is believed that the regulation of ROX1 may function as follows: According to Kwast et al., “Genomic Analysis of Anaerobically induced genes in Saccharomyces cerevisiae: Functional roles of ROX1 and other factors in mediating the anoxic response”, (2002), Journal of bacteriology vol 184, no 1 pages 250-265, herein incorporated by reference,: “Although Rox1 functions in an 02-independent manner, its expression is oxygen (heme) dependent, activated by the heme-dependent transcription factor Hap1 [19]. Thus, as oxygen levels fall to those that limit heme biosynthesis [20], ROX1 is no longer transcribed [21], its protein levels fall [22], and the genes it regulates are de-repressed”.

Further details and suitable motifs are provided by Keng, T. (1992), “HAP1 and ROX1 form a regulatory pathway in the repression of HEM13 transcription in Saccharomyces cerevisiae”, Mol. Cell. Biol. 12: pages 2616-2623, and Ter Kinde and de Steensma, “A microarray-assisted screen for potential Hap1 and Rox1 target genes in Saccharomyces cerevisiae”, (2002), Yeast 19: pages 825-840, incorporated herein by reference.

Preferably, the TKL promoter comprises a ROX1 binding motif. The TKL promoter may suitably comprise one or more ROX1 binding motif(s).

More preferably the TKL promoter can comprise in its nucleic acid sequence one or more copies of the motif NNNATTGTTNNN. Herein “N” represents a nucleic acid chosen from the group consisting of Adenine (A), Guanine (G), Cytosine (C) and Thymine (T). Such motif is illustrated by SEQ ID NO: 29.

More preferably, the TKL promoter comprises or consists of a nucleic acid sequence that is identical to the nucleic acid sequence of a, preferably native, promoter of a gene selected from the list consisting of: FET4, ANB1, YHR048W, DAN1, AAC3, TIR2, DIP5, HEM13, YNR014W, YAR028W, FUN 57, COX5B, OYE2, SUR2, FRDS1, PIS1, LAC1, YGR035C, YAL028W, EUG1, HEM14, ISU2, ERG26, YMR252C and SML1, more preferably FET4, ANB1, YHR048W, DAN1, AAC3, TIR2, DIP5 and HEM13, or a functional homologue thereof comprising a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The reference to a native promoter is herein to the promoter that is native to the host cell.

Preferably the recombinant yeast cell is a recombinant Saccharomyces cerevisiae yeast cell and preferably the TKL promoter is a native promoter of a Saccharomyces cerevisiae gene selected from the list consisting of: FET4, ANB1, YHR048W, DAN1, AAC3, TIR2, DIP5, HEM13, YNR014W, YAR028W, FUN 57, COX5B, OYE2, SUR2, FRDS1, PIS1, LAC1, YGR035C, YAL028W, EUG1, HEM14, ISU2, ERG26, YMR252C and SML1.

In addition or in the alternative, the TKL promoter preferably comprises in its nucleic acid sequence one or more copies of the motifs: TCGTTYAG and/or AAAAATTGTTGA. Herein “Y” represents C or T. The AAAAATTGTTGA motif is illustrated by SEQ ID NO: 30.

The TKL promoter can also comprise or consist of a nucleic acid sequence that is identical to the nucleic acid sequence of a, preferably native, promoter of a DAN, TIR or PAU gene. For example, the TKL promoter can suitably comprise or consist of a nucleic acid sequence of a, preferably native, promoter of a gene selected from the list consisting of: TIR2, DAN1, TIR4, TIR3, PAU7, PAU5, YLL064C, YGR294W, DAN3, YIL176C, YGL261C, YOL161C, PAU1, PAU6, DAN2, YDR542W, YIR041W, YKL224C, PAU3, YLL025W, YOR394W, YHL046C, YMR325W, YAL068C, YPL282C, PAU2, and PAU4 or a functional homologue thereof comprising a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith. The reference to a native promoter is herein to the promoter that is native to the host cell.

Preferably the recombinant yeast cell is a recombinant Saccharomyces cerevisiae yeast cell and preferably the TKL promoter is a native promoter of a Saccharomyces cerevisiae gene selected from the list consisting of: TIR2, DAN1, TIR4, TIR3, PAU7, PAU5, YLL064C, YGR294W, DAN3, YIL176C, YGL261C, YOL161C, PAU1, PAU6, DAN2, YDR542W, YIR041W, YKL224C, PAU3, YLL025W, YOR394W, YHL046C, YMR325W, YAL068C, YPL282C, PAU2, and PAU4.

More preferably, the TKL promoter can comprise or consist of a sequence that is identical to the nucleic acid sequence of a, preferably native, promoter of a gene selected from the list consisting of: TIR2, DAN1, TIR4, TIR3, PAU7, PAU5, YLL064C, YGR294W, DAN3, YIL176C, YGL261C, YOL161C, PAU1, PAU6, DAN2, YDR542W, YIR041 W, YKL224C, PAU3, and YLL025W or a functional homologue thereof comprising a nucleic acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity therewith.

The nucleic acid sequence of the S. cerevisiae ANB1 promoter is illustrated in SEQ ID NO: 31. The nucleic acid sequence of the S. cerevisiae DAN1 promoter is illustrated in SEQ ID NO: 32.

Preferred TKL promoters can thus comprise or consist of:
a nucleic acid sequence of SEQ ID NO: 31 or SEQ ID NO: 32; or
a functional homologue of the nucleic acid sequence of SEQ ID NO: 31 or SEQ ID NO: 32, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 31 or SEQ ID NO: 32; or
a functional homologue of the nucleic acid sequence of SEQ ID NO: 31 or SEQ ID NO: 32, having one or more mutations, substitutions, insertions and/or deletions as compared to the nucleic acid sequence of SEQ ID NO: 31 or SEQ ID NO: 32, wherein more preferably the nucleic acid sequence has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions as compared to the nucleic acid sequence of SEQ ID NO: 31 or SEQ ID NO: 32.

The TKL promoter can also be a synthetic oligonucleotide. That is, the TKL promoter may be a product of artificial oligonucleotide synthesis. Artificial oligonucleotide synthesis is a method in synthetic biology that is used to create artificial oligonucleotides, such as genes, in the laboratory. Commercial gene synthesis services are now available from numerous companies worldwide, some of which have built their business model around this task. Current gene synthesis approaches are most often based on a combination of organic chemistry and molecular biological techniques and entire genes may be synthesized “de novo”, without the need for precursor template DNA.

The TKL promoter has a TKL expression ratio _{anaerobic/aerobic} of 2 or more, preferably of 3 or more, 4 or more, 5 or more, 6 or more, 7 or more, 8 or more, 9 or more, 10 or more, 20 or more or 50 or more. By a TKL expression ratio _{anaerobic/aerobic} of 2 or more is suitably meant that the expression of the enzyme transketolase (“TKL”) is, under further identical expression conditions, at least a factor 2 higher under anaerobic conditions than under aerobic conditions.

There is no upper limit, and the TKL promoter can be a TKL promoter that allows the promoted transketolase gene to be expressed only at anaerobic conditions and not at aerobic conditions.

For practical reasons a TKL expression ratio _{anaerobic/aerobic} in the range from equal to or more than 2 to equal to or less than 10 exp 10 (i.e. 10¹⁰) or to or less than 10 exp 4 (i.e. 10⁴) can be considered.

As indicated above, “Expression” herein refers to the transcription of a gene into structural RNA (rRNA, tRNA) or messenger RNA (mRNA) with subsequent translation into a protein.

The TKL expression ratio can for example be determined by measuring the amount of Transketolase (TKL) protein of cells grown under aerobic and anaerobic conditions. The amount of TKL protein can be determined by proteomics or any other method known to quantify protein amounts.

It is also possible to determine the level or transketolase (TKL) expression ratio by measuring the transketolase (TKL) activity of cells grown under aerobic and anaerobic conditions, e.g. in a cell-free extract.

In addition or in the alternative to the above, the level or TKL expression ratio can be determined by measuring the transcription level (e.g. as amount of mRNA) of the TKL gene of cells grown under aerobic and anaerobic conditions. The skilled person knows how to determine translation levels using methods commonly known in the art, e.g. Q-PCR, real-time PCR, northern blot, RNA-seq.

The TKL promoter advantageously enables higher expression of transketolase during anaerobic conditions than under aerobic conditions. In the process according to the invention, the recombinant yeast cell preferably expresses transketolase, where the amount of transketolase expressed under anaerobic conditions is a multiplication factor higher than the amount of transketolase expressed under aerobic conditions and wherein this multiplication factor is preferably 2 or more, more preferably 3 or more, 4 or more, 5 or more, 6 or more, 7 or more, 8 or more, 9 or more, 10 or more, 20 or more or 50 or more.

### Increased Flux

Preferably the genetic modification(s) made in respect of the PPP-genes, i.e. with respect to TKL1 and optionally RKI, RPE and TAL, cause an increased flux of the non-oxidative part of the pentose phosphate pathway is herein understood to mean a modification that increases the flux by at least a factor of about 1.1, about 1.2, about 1.5, about 2, about 5, about 10 or about 20 as compared to the flux in a strain which is genetically identical except for the genetic modification causing the increased flux. The flux of the non-oxidative part of the pentose phosphate pathway may be measured by growing the modified host on xylose as sole carbon source, determining the specific xylose consumption rate and subtracting the specific xylitol production rate from the specific xylose consumption rate, if any xylitol is produced. However, the flux of the non-oxidative part of the pentose phosphate pathway is proportional with the growth rate on xylose as sole carbon source, preferably with the anaerobic growth rate on xylose as sole carbon source. There is a linear relation between the growth rate on xylose as sole carbon source (μₘₐₓ) and the flux of the non-oxidative part of the pentose phosphate pathway. The specific xylose consumption rate (Qₛ) is equal to the growth rate (μ) divided by the yield of biomass on sugar (Yₓₛ) because the yield of biomass on sugar is constant (under a given set of conditions: anaerobic, growth medium, pH, genetic background of the strain, etc.; i.e. Qₛ=μ/Yₓₛ). Therefore the increased flux of the non-oxidative part of the pentose phosphate pathway may be deduced from the increase in maximum growth rate under these conditions unless transport (uptake is limiting).

One or more genetic modifications that increase the flux of the pentose phosphate pathway may be introduced in the host cell in various ways. These including e.g. achieving higher steady state activity levels of xylulose kinase and/or one or more of the enzymes of the non-oxidative part pentose phosphate pathway and/or a reduced steady state level of unspecific aldose reductase activity. These changes in steady state activity levels may be effected by selection of mutants (spontaneous or induced by chemicals or radiation) and/or by recombinant DNA technology e.g. by overexpression or inactivation, respectively, of genes encoding the enzymes or factors regulating these genes.

In a preferred host cell, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate epimerase; or at least the enzymes ribulose-5-phosphate isomerase and transketolase; or at least the enzymes ribulose-5-phosphate isomerase and transaldolase; or at least the enzymes ribulose-5-phosphate epimerase and transketolase; or at least the enzymes ribulose-5-phosphate epimerase and transaldolase; or at least the enzymes transketolase and transaldolase; or at least the enzymes ribulose-5-phosphate epimerase, transketolase and transaldolase; or at least the enzymes ribulose-5-phosphate isomerase, transketolase and transaldolase; or at least the enzymes ribulose-5-phosphate isomerase, ribulose-5-phosphate epimerase, and transaldolase; or at least the enzymes ribulose-5-phosphate isomerase, ribulose-5-phosphate epimerase, and transketolase. In one embodiment of the invention each of the enzymes ribulose-5-phosphate isomerase, ribulose-5-phosphate epimerase, transketolase and transaldolase are overexpressed in the host cell. More preferred is a host cell in which the genetic modification comprises at least overexpression of both the enzymes transketolase and transaldolase as such a host cell is already capable of anaerobic growth on xylose. In fact, under some conditions host cells overexpressing only the transketolase and the transaldolase already have the same anaerobic growth rate on xylose as do host cells that overexpress all four of the enzymes, i.e. the ribulose-5-phosphate isomerase, ribulose-5-phosphate epimerase, transketolase and transaldolase. Moreover, host cells overexpressing both of the enzymes ribulose-5-phosphate isomerase and ribulose-5-phosphate epimerase are preferred over host cells overexpressing only the isomerase or only the epimerase as overexpression of only one of these enzymes may produce metabolic imbalances.

### Rubisco

The recombinant yeast cell is suitably functionally expressing one or more heterologous nucleic acid sequences encoding for ribulose-1,5-phosphate carboxylase/oxygenase (EC4.1.1.39; Rubisco), and optionally one or more molecular chaperones for Rubisco.

More preferably the recombinant yeast cell functionally expresses:
a heterologous nucleic acid sequence encoding a protein having ribulose-1,5-biphosphate carboxylase oxygenase (Rubisco) activity; and/or
a heterologous nucleic acid sequence encoding a protein having phosphoribulokinase (PRK) activity; and/or
optionally one or more heterologous nucleic acid sequence encoding one or more molecular chaperones for the protein having ribulose-1,5-biphosphate carboxylase oxygenase (Rubisco) activity.

The protein having ribulose-1,5-biphosphate carboxylase oxygenase (Rubisco) activity is herein also referred to as “ribulose-1,5-biphosphate carboxylase oxygenase”, “ribulose-1,5-biphosphate carboxylase oxygenase protein”, “ribulose-1,5-biphosphate carboxylase oxygenase enzyme”, “Rubisco enzyme”, “Rubisco protein” or simply “Rubisco”. A ribulose-1,5-biphosphate carboxylase oxygenase may be further defined by its amino acid sequence. Likewise a ribulose-1,5-biphosphate carboxylase oxygenase may be further defined by a nucleotide sequence encoding the ribulose-1,5-biphosphate carboxylase oxygenase. As explained in detail above under definitions, a certain ribulose-1,5-biphosphate carboxylase oxygenase that is defined by a nucleotide sequence encoding the enzyme, includes (unless otherwise limited) the nucleotide sequence hybridising to such nucleotide sequence encoding the ribulose-1,5-biphosphate carboxylase oxygenase. Preferences for the Rubisco protein and the nucleic sequences encoding for such are as described in WO2014/129898, incorporated herein by reference.

The Rubisco protein may suitably be selected from the group of eukaryotic and prokaryotic Rubisco proteins. The Rubisco protein is preferably from a non-phototrophic organism. For example, the Rubisco protein may be from a chemolithoautotrophic microorganism. Good results have been achieved with a bacterial Rubisco protein. Preferably, the Rubisco protein originates from a Thiobacillus, in particular, Thiobacillus denitrificans, which is chemolithoautotrophic.

The Rubisco protein may be a single-subunit Rubisco protein or a Rubisco protein having more than one subunit. Preferably the Rubisco protein is a single-subunit Rubisco protein. Good results have been obtained with a Rubisco protein that is a so-called form-II Rubisco protein. Especially good results were achieved with a Rubisco protein encoded by a cbbM gene, also referred to as CbbM.

A preferred Rubisco protein is the Rubisco protein encoded by the cbbM gene from Thiobacillus denitrificans. SEQ ID NO: 1 shows the amino acid sequence of a suitable Rubisco protein, encoded by the cbbM gene from Thiobacillus denitrificans. SEQ ID NO: 2 illustrates the nucleic acid sequence of the cbbM gene from Thiobacillus denitrificans, codon optimized for S. cerevisiae.

Preferably the protein having ribulose-1,5-biphosphate carboxylase oxygenase (Rubisco) activity thus comprises or consists of:
an amino acid sequence of SEQ ID NO: 1; or
a functional homologue of SEQ ID NO: 1, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 1; or
a functional homologue of SEQ ID NO: 1, having one or more mutations, substitutions, insertions and/or deletions when compared with the amino acid sequence of SEQ ID NO: 1, more preferably a functional homologue that has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions when compared with the amino acid sequence of SEQ ID NO: 1.

Preferable the nucleic acid sequence encoding the protein having ribulose-1,5-biphosphate carboxylase oxygenase (Rubisco) activity comprises or consists of:
a nucleic acid sequence of SEQ ID NO: 2; or
a functional homologue of SEQ ID NO: 2, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 2; or
a functional homologue of SEQ ID NO: 2, having one or more mutations, substitutions, insertions and/or deletions when compared with the nucleic acid sequence of SEQ ID NO: 2, more preferably a functional homologue that has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions when compared with the nucleic acid sequence of SEQ ID NO: 2.

Examples of other suitable Rubisco polypeptides and their origin are given in Table 1 of WO2014/129898, incorporated herein by reference, and in Table 2 below, with reference to the sequence identity with the amino acid sequence of SEQ ID NO:1.

The nucleic acid sequence (e.g. the gene) encoding for the ribulose-1,5-biphosphate carboxylase oxygenase (Rubisco) protein may suitably be incorporated in the genome of the recombinant yeast cell, for example as described in the examples of WO2014/129898 and by the article of Guadalupe-Medina et al., “Carbon dioxide fixation by Calvin-Cycle enzymes improves ethanol yield in yeast”, published in Biotechnol, Biofuels, 2013, vol. 6, p. 125, both herein incorporated by reference.

TABLE 2
Natural Rubisco polypeptides suitable for expression with sequence
identity to Rubisco polypeptide of Thiobacillus denitrificans.

| | | MAX ID |
|---|---|---|
| Source | Accession no. | (%) |
| Thiobacillus denitrificans | AAA99178.2 | 100 |
| Sideroxydans lithotrophicus ES-1 | YP_003522651.1 | 94 |
| Thiothrix nivea DSM 5205 | ZP_10101642.1 | 91 |
| Halothiobacillus neapolitanus c2 | YP_003262978.1 | 90 |
| Acidithiobacillus ferrooxidans | YP_002220242.1 | 88 |
| ATCC 53993 | | |
| Rhodoferax ferrireducens T118 | YP_522655.1 | 86 |
| Thiorhodococcus drewsii AZ1 | ZP_08824342.1 | 85 |
| uncultured prokaryote | AGE14067.1 | 82 |

As indicated above, the Rubisco protein is suitably functionally expressed in the recombinant yeast cell, at least during use in a fermentation process.

The nucleic acid sequence encoding for the Rubisco protein can be present in one, two or more copies with the recombinant yeast cell. Without wishing to be bound by any kind of theory it is believed that the robustness of the recombinant yeast cell is best served when the nucleic acid sequence (e.g. the gene) encoding for the Rubisco protein is present in the recombinant yeast cell in less than 12 copies, more preferably less than 8 copies. Preferably the recombinant yeast cell therefore comprises in the range from equal to or more than 1 copy, more preferably equal to or more than 2 copies, to equal to or less than 7 copies, more preferably equal to or less than 6 copies of a nucleic acid sequence (e.g. a gene) encoding for a Rubisco protein. The recombinant yeast cell may for example comprise one, two, three, four, five, six or seven copies of a nucleic acid sequence encoding for ribulose-1,5-biphosphate carboxylase oxygenase (Rubisco).

To increase the likelihood that the Rubisco protein is expressed at sufficient levels and in active form in the transformed (recombinant) host cells of the invention, the nucleic acid sequence encoding the Rubisco protein and other proteins as described herein (see below), are preferably adapted to optimise their codon usage to that of the host cell in question. The adaptiveness of a nucleic acid sequence encoding an enzyme to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, “The codon adaptation index—a measure of directional synonymous codon usage bias, and its potential applications”, (1987), published in Nucleic Acids Research vol. 15, pages 1281-1295; also see: Jansen et al., “Revisiting the codon adaptation index from a whole-genome perspective: analyzing the relationship between gene expression and codon occurrence in yeast using a variety of models”, (2003), Nucleic Acids Res. Vol. 31(8), pages 2242-51). An adapted nucleic acid sequence preferably has a CAI of at least 0.2, 0.3, 0.4, 0.5, 0.6, 0.7, 0.8 or 0.9. Preferably, the sequences have been codon optimized for expression in the fungal host cell in question, such as for example Saccharomyces cerevisiae cells. Preferably, the sequences have been codon optimized for expression in the fungal host cell in question, such as for example Saccharomyces cerevisiae cells.

Preferably the functionally expressed Rubisco protein has an activity, defined by the rate of ribulose-1,5-bisphosphate-dependent ¹⁴C-bicarbonate incorporation by cell extracts of at least 1 nmol·min⁻¹·(mg protein)⁻¹, in particular an activity of at least 2 nmol·min⁻¹·(mg protein)⁻¹, more in particular an activity of at least 4 nmol·min⁻¹·(mg protein)⁻¹. The upper limit for the activity is not critical. In practice, the activity may be about 200 nmol·min⁻¹·(mg protein)⁻¹ or less, in particular 25 nmol·min⁻¹·(mg protein)⁻¹, more in particular 15 nmol·min⁻¹·(mg protein)⁻¹ or less, e.g. about 10 nmol·min⁻¹·(mg protein)⁻¹ or less. The conditions for an assay for determining this Rubisco activity are as found in the Examples (e.g. Example 4) of WO2014/129898, incorporated herein by reference.

### Phosphoribulokinase

Suitably, the recombinant yeast cell is also functionally expressing one or more, preferably heterologous, nucleic acid sequences encoding a protein having phosphoribulokinase (PRK) activity (EC2.7.1.19).

The protein having phosphoribulokinase (PRK) activity is herein also referred to as “phosphoribulokinase protein”, “phosphoribulokinase enzyme”, “phosphoribulokinase”, “PRK enzyme”, “PRK protein” or simply “PRK”. Preferences for the PRK protein and the nucleic sequences encoding for such are as described in WO2014/129898, incorporated herein by reference.

A functionally expressed phosphoribulokinase (PRK, (EC 2.7.1.19)) according to the invention is capable of catalyzing the chemical reaction:

ATP+D-ribulose 5-phosphate [symbol] ADP+D-ribulose 1,5-bisphosphate

Thus, the two substrates of this enzyme are ATP and D-ribulose 5-phosphate; its two products are ADP and D-ribulose 1,5-bisphosphate.

The PRK protein belongs to the family of transferases, specifically those transferring phosphorus-containing groups (phosphotransferases) with an alcohol group as acceptor. The systematic name of this enzyme class is ATP:D-ribulose-5-phosphate 1-phosphotransferase. Other names in common use include phosphopentokinase, ribulose-5-phosphate kinase, phosphopentokinase, phosphoribulokinase (phosphorylating), 5-phosphoribulose kinase, ribulose phosphate kinase, PKK, PRuK, and PRK. The PRK enzyme participates in carbon fixation. A phosphoribulokinase (PRK) protein may be further defined by its amino acid sequence. Likewise a phosphoribulokinase (PRK) protein may be further defined by a nucleotide sequence encoding the phosphoribulokinase (PRK). As explained in detail above under definitions, a certain phosphoribulokinase (PRK) that is defined by a nucleotide sequence encoding the enzyme, includes (unless otherwise limited) the nucleotide sequence hybridising to such nucleotide sequence encoding the phosphoribulokinase (PRK).

The PRK can be from a prokaryote or a eukaryote. Good results have been achieved with a PRK originating from a eukaryote. Preferably the PRK protein originates from a plant selected from Caryophyllales, in particular from Amaranthaceae, more in particular from Spinacia.

A preferred PRK protein is the PRK protein from Spinacia. SEQ ID NO: 3 shows the amino acid sequence of such PRK protein from Spinacia. SEQ ID NO: 4 illustrates the nucleic acid sequence of the prk gene from Spinacia oleracea—codon optimized for S. cerevisiae.

Preferably the protein having phosphoribulokinase (PRK) activity thus comprises or consists of:
an amino acid sequence of SEQ ID NO: 3; or
a functional homologue of SEQ ID NO: 3, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 3; or
a functional homologue of SEQ ID NO: 3, having one or more mutations, substitutions, insertions and/or deletions when compared with the amino acid sequence of SEQ ID NO: 3, more preferably a functional homologue that has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions when compared with the amino acid sequence of SEQ ID NO: 3.

Preferable the nucleic acid sequence encoding the protein having phosphoribulokinase (PRK) activity comprises or consists of:
a nucleic acid sequence of SEQ ID NO: 4; or
a functional homologue of SEQ ID NO: 4, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 4; or
a functional homologue of SEQ ID NO: 4, having one or more mutations, substitutions, insertions and/or deletions when compared with the nucleic acid sequence of SEQ ID NO: 4, more preferably a functional homologue that has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions when compared with the nucleic acid sequence of SEQ ID NO: 4.

The nucleic acid sequence (e.g. the gene) encoding for the protein having phosphoribulokinase (PRK) activity may suitably be incorporated in the genome of the recombinant yeast cell, for example as described in the examples of WO2014/129898, herein incorporated by reference.

Examples of suitable PRK polypeptides and their origin are given in Table 2 of WO2014/129898, incorporated herein by reference, and in Table 3 below, with reference to the sequence identity with the amino acid sequence of SEQ ID NO:3.

TABLE 3
Natural PRK polypeptides suitable for expression
with identity to PRK from Spinacia

| Source | Accession no. | MAX ID (%) |
|---|---|---|
| Spinacia oleracea | P09559.1 | 100 |
| Medicago truncatula | XP_003612664.1 | 88 |
| Arabidopsis thaliana | NP_174486.1 | 87 |
| Vitis vinifera | XP_002263724.1 | 86 |
| Closterium peracerosum | BAL03266.1 | 82 |
| Zea mays | NP_001148258.1 | 78 |

The nucleic acid sequences encoding for the PRK protein may be under the control of a promoter (the “PRK promoter”) that enables higher expression under anaerobic conditions than under aerobic conditions. Examples of such promoters are described in WO2017/216136A1 and WO2018/228836, both herein incorporated by reference. More preferably such promoter has a PRK expression ratio anaerobic/aerobic of 2 or more, 3 or more, 4 or more, 5 or more, 6 or more, 7 or more, 8 or more, 9 or more, 10 or more, 20 or more or 50 or more. Further preferences are as described in WO2018/228836, incorporated herein by reference.

### Rubisco Chaperones

Optionally, the recombinant yeast cell further comprises one or more, preferably heterologous, nucleic acid sequences encoding for one or more molecular chaperones for the protein having ribulose-1,5-biphosphate carboxylase oxygenase (Rubisco) activity.

Suitably such molecular chaperones are also referred herein as “chaperone protein”, “chaperonin” or simply “chaperone”. Preferences for the chaperones and the nucleic sequences encoding for such are as described in WO2014/129898, incorporated herein by reference.

Preferably the recombinant yeast cell comprises one or more heterologous nucleic acid sequences encoding for one or more molecular chaperones for the protein having ribulose-1,5-biphosphate carboxylase oxygenase (Rubisco) activity.

Chaperonins are proteins that provide favorable conditions for the correct folding of other proteins, thus preventing aggregation. Newly made proteins usually must fold from a linear chain of amino acids into a three-dimensional form. Chaperonins belong to a large class of molecules that assist protein folding, called molecular chaperones. The energy to fold proteins is supplied by adenosine triphosphate (ATP). A review article about chaperones that is useful herein is written by Yébenes et al., “Chaperonins: two rings for folding” (2011), Trends in Biochemical Sciences, Vol. 36, No. 8, pages 424-432, incorporated herein by reference.

The chaperone or chaperones may be prokaryotic chaperones or eukaryotic chaperones. In addition, the chaperones may be homologous or heterologous. For example, the recombinant yeast cell may comprises one or more nucleic acid sequence encoding one or more homologous or heterologous, prokaryotic or eukaryotic, molecular chaperones, which—when expressed—are capable of functionally interacting with an enzyme in the recombinant yeast cell, in particular with at least one of Rubisco and PRK.

Suitably the chaperone or chaperones are derived from a bacterium, more preferably from Escherichia, in particular E. coli. Preferred chaperones are GroEL and GroEs from E. coli. Other preferred chaperones are chaperones from Saccharomyces, in particular Saccharomyces cerevisiae Hsp10 and Hsp60.

If the chaperones are naturally expressed in an organelle such as a mitochondrion (examples are Hsp60 and Hsp10 of Saccharomyces cerevisiae) relocation to the cytosol can be achieved e.g. by modifying the native signal sequence of the chaperonins. In eukaryotes the proteins Hsp60 and Hsp10 are structurally and functionally nearly identical to GroEL and GroES, respectively. Thus, it is contemplated that Hsp60 and Hsp10 from any recombinant yeast cell may serve as a chaperone for the Rubisco. This is described for example by Zeilstra-Ryalls et al., “The universally conserved GroE (Hsp60) chaperonins”, published in Annu Rev Microbiol. (1991) vol. 45, pages 301-25; and Horwich et al., “Two Families of Chaperonin: Physiology and Mechanism”, published in Annu. Rev. Cell. Dev. Biol. Vol. 23, pages 115-45, both herewith incorporated by reference.

Good results have been achieved with a recombinant yeast cell comprising both the heterologous chaperones GroEL and GroES.

As an alternative to GroES a functional homologue of GroES may be present, preferably a functional homologue comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of GroES, respectively the amino acid sequence of SEQ ID NO: 7.

SEQ ID NO:7 provides a preferred translated protein sequence, based on GroES of Escherichia coli. SEQ ID NO: 8 provides a synthetic nucleic acid sequence, based on GroES from Escherichia coli, codon optimized for expression in Saccharomyces cerevisiae.

Examples of suitable natural chaperones polypeptide homologous to GroES are given in Table 4.

TABLE 4
Natural chaperones homologous to GroES polypeptides suitable for expression

>gi|115388105|ref|XP_001211558.1|:2-101 10 kDa heat shock protein, mitochondrial
[Aspergillus terreus NIH2624]
>gi|116196854|ref|XP_001224239.1|:1-102 conserved hypothetical protein [Chaetomium
globosum CBS 148.51]
>gi|119175741|ref|XP_001240050.1|:3-102 hypothetical protein CIMG_09671 [Coccidioides
immitis RS]
>gi|119471607|ref|XP_001258195.1|:12-111 chaperonin, putative [Neosartorya fischeri
NRRL181]
>gi|121699818|ref|XP_001268174.1|:8-106 chaperonin, putative [Aspergillus clavatus NRRL1]
>gi|126274604|ref|XP_001387607.1|:2-102 predicted protein [Scheffersomyces stipitis CBS
6054]
>gi|146417701|ref|XP_001484818.1|:5-106 conserved hypothetical protein [Meyerozyma
guilliermondii ATCC 6260]
>gi|154303611|ref|XP_001552212.1|:1-102 10 kDa heat shock protein, mitochondrial
[Botryotinia fuckeliana B05.10]
>gi|156049571|ref|XP_001590752.1|:1-102 hypothetical protein SS1G_08492 [Sclerotinia
sclerotiorum 1980]
>gi|156840987|ref|XP_001643870.1|:1-103 hypothetical protein KpoI_495p10
[Vanderwaltozyma polyspora DSM 70924]
>gi|169608295|ref|XP_001797567.1|:1-101 hypothetical protein SNOG_07218
[Phaeosphaeria nodorum SN15]
>gi|171688384|ref|XP_001909132.1|:1-102 hypothetical protein [Podospora anserina S mat+]
>gi|189189366|ref|XP_001931022.1|:71-168 10 kDa chaperonin [Pyrenophora tritici-repentis
Pt-1C-BFP]
>gi|19075598|ref|NP_588098.1|:1-102 mitochondrial heat shock protein Hsp10 (predicted)
[Schizosaccharomyces pombe 972h-]
>gi|212530240|ref|XP_002145277.1|:3-100 chaperonin, putative [Talaromyces marneffei
ATCC 18224]
>gi|212530242|ref|XP_002145278.1|:3-95 chaperonin, putative [Talaromyces marneffei
ATCC 18224]
>gi|213404320|ref|XP_002172932.1|:1-102 mitochondrial heat shock protein Hsp10
[Schizosaccharomyces japonicus yFS275]
>gi|225557301|gb|EEH05587.1|:381-478 pre-mRNA polyadenylation factor fip1 [Ajellomyces
capsulatus G186AR]
>gi|225684092|gb|EEH22376.1|:3-100 heat shock protein [Paracoccidioides brasiliensis
Pb03
>gi|238490530|ref|XP_002376502.1|:2-104 chaperonin, putative [Aspergillus flavus
NRRL3357
>gi|238878220|gb|EEQ41858.1|:1-106 10 kDa heat shock protein, mitochondrial [Candida
albicans WO-1]
>gi|240280207|gb|EER43711.1|:426-523 pre-mRNA polyadenylation factor fip1 [Ajellomyces
capsulatus H143]
>gi|241950445|ref|XP_002417945.1|:1-103 10 kda chaperonin, putative; 10 kda heat shock
protein mitochondrial (hsp10), putative [Candida dubliniensis CD36]
>gi|242819222|ref|XP_002487273.1|:90-182 chaperonin, putative [Talaromyces stipitatus
ATC
>gi|254566327|ref|XP_002490274.1|:1-102 Putative protein of unknown function
[Komagataella pastoris GS115]
>gi|254577241|ref|XP_002494607.1|:1-103 ZYRO0A05434p [Zygosaccharomyces rouxii]
>gi|255717999|ref|XP_002555280.1|:1-103 KLTH0G05588p [Lachancea thermotolerans]
>gi|255956581|ref|XP_002569043.1|:2-101 Pc21g20560 [Penicillium chrysogenum
Wisconsin 54-1255]
>gi|258572664|ref|XP_002545094.1|:16-108 chaperonin GroS [Uncinocarpus reesii 1704]
>gi|261190594|ref|XP_002621706.1|:3-100 chaperonin [Ajellomyces dermatitidis SLH14081]
>gi|295664909|ref|XP_002793006.1|:3-100 10 kDa heat shock protein, mitochondrial
[Paracoccidioides sp. ‘lutzii’Pb01]
>gi|296412657|ref|XP_002836039.1|:76-177 hypothetical protein [Tuber melanosporum
Mel28]
>gi|302307854|ref|NP_984626.2|:2-102 AEL235Wp [Ashbya gossypii ATCC 10895]
>gi|302894117|ref|XP_003045939.1|:1-102 predicted protein [Nectria haematococca mpVI
77-13-4]
>gi|303318351|ref|XP_003069175.1|:3-100 10 kDa heat shock protein, mitochondrial,
putative [Coccidioides posadasii C735 delta SOWgp]
>gi|310795300|gb|EFQ30761.1|:1-102 chaperonin 10 kDa subunit [Glomerella graminicola
M1.001]
>gi|315053085|ref|XP_003175916.1|:12-109 chaperonin GroS [Arthroderma gypseum CBS
118893]
>gi|317032114|ref|XP_001394060.2|:334-433 heat shock protein [Aspergillus niger CBS
513.88]
>gi|317032116|ref|XP_001394059.2|:2-101 heat shock protein [Aspergillus niger CBS
513.88]
>gi|320583288|gb|EFW97503.1|:6-106 chaperonin, putative heat shock protein, putative
[Ogataea parapolymorpha DL-1]
>gi|320591507|gb|EFX03946.1|:1-102 heat shock protein [Grosmannia clavigera kw1407]
>gi|322700925|gb|EFY92677.1|:1-102 chaperonin [Metarhizium acridum CQMa 102]
>gi|325096696|gb|EGC50006.1|:409-506 pre-mRNA polyadenylation factor fip1 [Ajellomyces
capsulatus H88]
>gi|326471604|gb|EGD95613.1|:14-111 chaperonin 10 Kd subunit [Trichophyton tonsurans
CBS112818]
>gi|327293056|ref|XP_003231225.1|:3-100 chaperonin [Trichophyton rubrum CBS 118892]
>gi|330942654|ref|XP_003306155.1|:37-136 hypothetical protein PTT_19211 [Pyrenophora
teres f. teres 0-1]
>gi|336268042|ref|XP_003348786.1|:47-147 hypothetical protein SMAC_01809 [Sordaria
macrospora khell]
>gi|340519582|gb|EGR49820.1|:1-109 predicted protein [Trichoderma reesei QM6a]
>gi|340960105|gb|EGS21286.1|:3-103 putative mitochondrial 10 kDa heat shock protein
[Chaetomium thermophilum var. thermophilum DSM 1495]
>gi|342883802|gb|EGU84224.1|:1-102 hypothetical protein FOXB_05181 [Fusarium
oxysporum Fo5176]
>gi|344302342|gb|EGW32647.1|:2-102 hypothetical protein SPAPADRAFT_61712
[Spathaspora passalidarum NRRL Y-27907]
>gi|345570750|gb|EGX53571.1|:1-102 hypothetical protein AOL_s00006g437 [Arthrobotrys
oligospora ATCC 24927]
>gi|346321154|gb|EGX90754.1|:1-102 chaperonin [Cordyceps militaris CM01]
>gi|346970393|gb|EGY13845.1|:1-102 heat shock protein [Verticillium dahliae VdLs.17]
>gi|354548296|emb|CCE45032.1|:1-106 hypothetical protein CPAR2_700360 [Candida
parapsilosis]
>gi|358385052|gb|EHK22649.1|:1-102 hypothetical protein TRIVIDRAFT_230640
[Trichoderma virens Gv 29-8]
>gi|358393422|gb|EHK42823.1|:1-101 hypothetical protein TRIATDRAFT_258186
[Trichoderma atroviride IMI 206040]
>gi|361126733|gb|EHK98722.1|:1-97 putative 10 kDa heat shock protein, mitochondrial
[Glare lozoyensis 74030]
>gi|363753862|ref|XP_003647147.1|:2-102 hypothetical protein Ecym_5593 [Eremothecium
cymbalariae DBVPG#7215]
>gi|365758401|gb|EHN00244.1|:1-106 Hsp10p [Saccharomyces cerevisiae ×
Saccharomyces kudriavzevii VIN7]
>gi|365987664|ref|XP_003670663.1|:1-103 hypothetical protein NDAI_0F01010
[Naumovozyma dairenensis CBS 421]
>gi|366995125|ref|XP_003677326.1|:1-103 hypothetical protein NCAS_0G00860
[Naumovozyma castellii CBS 4309]
>gi|366999797|ref|XP_003684634.1|:1-103 hypothetical protein TPHA_0C00430
[Tetrapisispora phaffii CBS 4417]
>gi|367009030|ref|XP_003679016.1|:1-103 hypothetical protein TDEL_0A04730
[Torulaspora delbruekii]
>gi|367023138|ref|XP_003660854.1|:1-104 hypothetical protein MYCTH_59302
[Myceliophthora thermophila ATCC 42464]
>gi|367046344|ref|XP_003653552.1|:1-102 hypothetical protein THITE_2116070
[Thielavia terrestris NRRL8126]
>gi|378726440|gb|EHY52899.1|:9-109 chaperonin GroES [Exophiala dermatitidis
NIH/UT8656]
>gi|380493977|emb|CCF33483.1|:1-102 chaperonin 10 kDa subunit [Colletotrichum
higginsianu
>gi|385305728|gb|EIF49680.1|:1-102 10 kda heat shock mitochondrial [Dekkera bruxellensis
AWRI1499]
>gi|389628546|ref|XP_003711926.1|:1-102 hsp10-like protein [Magnaporthe oryzae 70-15]
>gi|396462608|ref|XP_003835915.1|:1-101 similar to 10 kDa heat shock protein
[Leptosphaeria maculans JN3]
>gi|398392541|ref|XP_003849730.1|:1-102 hypothetical protein MYCGRDRAFT_105721
[Zymoseptoria tritici IPO323]
>gi|400597723|gb|EJP65453.1|:24-124 chaperonin 10 kDa subunit [Beauveria bassiana
ARSEF 2860]
>gi|401623646|gb|EJS41738.1|:1-106 hsp10p [Saccharomyces arboricola H-6]
>gi|401842164|gb|EJT44422.1|:1-92 HSP10-like protein [Saccharomyces kudriavzevii IFO
1802]
>gi|402084027|gb|EJT79045.1|:1-102 hsp10-like protein [Gaeumannomyces graminis var.
triti
>gi|403215209|emb|CCK69709.1|:1-104 hypothetical protein KNAG_0C06130 [Kazachstania
naganishii CBS 8797]
>gi|406604629|emb|CCH43969.1|:4-100 hypothetical protein BN7_3524 [Wickerhamomyces
ciferrii]
>gi|406867021|gb|EKD20060.1|:56-156 hypothetical protein MBM_02012 [Marssonina
brunnea f. sp. ‘multigermtubi’ MB_m1]
>gi|407926227|gb|EKG19196.1|:74-174 GroES-like protein [Macrophomina phaseolina MS6]
>gi|408398157|gb|EKJ77291.1|:11-111 hypothetical protein FPSE_02566 [Fusarium
pseudograminearum CS3096]
>gi|410082063|ref|XP_003958610.1|:1-103 hypothetical protein KAFR_0H00660
[Kazachstania africana CBS2517]
>gi|425777664|gb|EKV15823.1|:58-157 Chaperonin, putative [Penicillium digitatum Pd1]
>gi|440639680|gb|ELR09599.1|:1-102 chaperonin GroES [Geomyces destructans 20631-21]
>gi|444323906|ref|XP_004182593.1|:1-105 hypothetical protein TBLA_0J00760
[Tetrapisisporablattae CBS 6284]
>gi|448083208|ref|XP_004195335.1|:2-101 Piso0_005888 [Millerozyma farinosa CBS 7064]
>gi|448087837|ref|XP_004196425.1|:2-102 Piso0_005888 [Millerozyma farinosa CBS 7064]
>gi|448534948|ref|XP_003870866.1|:1-106 Hsp10 protein [Candida orthopsilosis Co 90-125]
>gi|449295977|gb|EMC91998.1|:1-102 hypothetical protein BAUCODRAFT_39148
[Baudoinia compn
>gi|46123659|ref|XP_386383.1|:3-103 hypothetical protein FG06207.1 [Gibberella zeae PH-1]
>gi|50289455|ref|XP_447159.1|:1-103 hypothetical protein [Candida glabrata CBS 138]
>gi|50308731|ref|XP_454370.1|:1-103 hypothetical protein [Kluyveromyces lactis NRRL
Y-1140]
>gi|50411066|ref|XP_457014.1|:1-106 DEHA2B01122p [Debaryomyces hansenii CBS767]
>gi|50545998|ref|XP_500536.1|:1-102 YALI0B05610p [Yarrowia lipolytica]
>gi|51013895|gb|AAT93241.1|:1-106 YOR020C [Saccharomyces cerevisiae]
>gi|6324594|ref|NP_014663.1|:1-106 Hsp10p [Saccharomyces cerevisiae S288c]
>gi|67523953|ref|XP_660036.1|:2-101 hypothetical protein AN2432.2 [Aspergillus nidulans
FGSC A4]
>gi|70992219|ref|XP_750958.1|:12-106 chaperonin [Aspergillus fumigatus Af293]
>gi|85079266|ref|XP_956315.1|:1-104 hypothetical protein NCU04334 [Neurospora crassa
OR74A]

As an alternative to GroEL a functional homologue of GroEL may be present, in particular a functional homologue comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of GroEL, respectively the amino sequence of SEQ ID NO: 5

SEQ ID NO:5 provides a preferred translated protein sequence, based on GroEL of Escherichia coli. SEQ ID NO: 6 provides a synthetic nucleic acid sequence, based on GroEL from Escherichia coli, codon optimized for expression in Saccharomyces cerevisiae.

Examples of suitable natural chaperones polypeptides homologous to GroEL are given in Table 5.

TABLE 5
Natural chaperones homologous to GroEL polypeptides suitable for expression

>gi|115443330|ref|XP_001218472.1| heat shock protein 60, mitochondrial precursor
[Aspergillus terreus NIH2624]
>gi|114188341|gb|EAU30041.1| heat shock protein 60, mitochondrial precursor [Aspergillus
terreus NIH2624]
>gi|119480793|ref|XP_001260425.1| antigenic mitochondrial protein HSP60, putative
[Neosartorya fischeri NRRL 181] >gi|119408579|gb|EAW18528.1| antigenic mitochondrial
protein HSP60, putative [Neosartorya fischeri NRRL 181]
>gi|126138730|ref|XP_001385888.1| hypothetical protein PICST_90190 [Scheffersomyces
stipitis CBS 6054] >gi|126093166|gb|ABN67859.1| mitochondrial groEL-type heat shock
protein [Scheffersomyces stipitis CBS 6054]
>gi|145246630|ref|XP_001395564.1| heat shock protein 60 [Aspergillus niger CBS 513.88]
>gi|134080285|emb|CAK46207.1| unnamed protein product [Aspergillus niger]
>gi|350636909|gb|EHA25267.1| hypothetical protein ASPNIDRAFT_54001 [Aspergillus niger
ATCC 1015]
>gi|146413148|ref|XP_001482545.1| heat shock protein 60, mitochondrial precursor
[Meyerozyma guilliermondii ATCC 6260]
>gi|154277022|ref|XP_001539356.1| heat shock protein 60, mitochondrial precursor
[Ajellomyces capsulatus NAm1] >gi|150414429|gb|EDN09794.1| heat shock protein 60,
mitochondrial precursor [Ajellomyces capsulatus NAm1]
>gi|154303540|ref|XP_001552177.1| heat shock protein 60 [Botryotinia fuckeliana B05.10]
>gi|347840915|emb|CCD55487.1| similar to heat shock protein 60 [Botryotinia fuckeliana]
>gi|156063938|ref|XP_001597891.1| heat shock protein 60, mitochondrial precursor
[Sclerotinia sclerotiorum 1980] >gi|154697421|gb|EDN97159.1| heat shock protein 60,
mitochondrial precursor [Sclerotinia sclerotiorum 1980 UF-70]
>gi|156844469|ref|XP_001645297.1| hypothetical protein KpoI_1037p35 [Vanderwaltozyma
polyspora DSM 70294] >gi|156115957|gb|EDO17439.1| hypothetical protein KpoI_1037p35
[Vanderwaltozyma polyspora DSM 70294]
>gi|16416029|emb|CAB91379.2| probable heat-shock protein hsp60 [Neurospora crassa]
>gi|350289516|gb|EGZ70741.1| putative heat-shock protein hsp60 [Neurospora tetrasperma
FGSC 2509]
>gi|169626377|ref|XP_001806589.1| hypothetical protein SNOG_16475 [Phaeosphaeria
nodorum SN15] >gi|111055053|gb|EAT76173.1| hypothetical protein SNOG_16475
[Phaeosphaeria nodorum SN15]
>gi|169783766|ref|XP_001826345.1| heat shock protein 60 [Aspergillus oryzae RIB40]
>gi|238493601|ref|XP_002378037.1| antigenic mitochondrial protein HSP60, putative
[Aspergillus flavus NRRL3357] >gi|83775089|dbj|BAE65212.1| unnamed protein product
[Aspergillus oryzae RIB40] >gi|220696531|gb|EED52873.1| antigenic mitochondrial protein
HSP60, putative [Aspergillus flavus NRRL3357] >gi|391869413|gb|EIT78611.1| chaperonin,
Cpn60/Hsp60p [Aspergillus oryzae 3.042]
>gi|189190432|ref|XP_001931555.1| heat shock protein 60, mitochondrial precursor
[Pyrenophora tritici-repentis Pt-1C-BFP] >gi|187973161|gb|EDU40660.1| heat shock protein
60, mitochondrial precursor [Pyrenophora tritici-repentis Pt-1C-BFP]
>gi|190348913|gb|EDK41467.2| heat shock protein 60, mitochondrial precursor [Meyerozyma
guilliermondii ATCC 6260]
>gi|225554633|gb|EEH02929.1| hsp60-like protein [Ajellomyces capsulatus G186AR]
>gi|238880068|gb|EEQ43706.1| heat shock protein 60, mitochondrial precursor [Candida
albicans WO-1]
>gi|239613490|gb|EEQ90477.1| chaperonin GroL [Ajellomyces dermatitidis ER-3]
>gi|240276977|gb|EER40487.1| hsp60-like protein [Ajellomyces capsulatus H143]
>gi|241958890|ref|XP_002422164.1| heat shock protein 60, mitochondrial precursor, putative
[Candida dubliniensis CD36] >gi|223645509|emb|CAX40168.1| heat shock protein 60,
mitochondrial precursor, putative [Candida dubliniensis CD36]
>gi|254572906|ref|XP_002493562.1| Tetradecameric mitochondrial chaperonin [Komagataella
pastoris GS115] >gi|238033361|emb|CAY71383.1| Tetradecameric mitochondrial chaperonin
[Komagataella pastoris GS115]
>gi|254579947|ref|XP_002495959.1| ZYRO0C07106p [Zygosaccharomyces rouxii]
>gi|238938850|emb|CAR27026.1| ZYRO0C07106p [Zygosaccharomyces rouxii]
>gi|255712781|ref|XP_002552673.1| KLTH0C10428p [Lachancea thermotolerans]
>gi|238934052|emb|CAR22235.1| KLTH0C10428p [Lachancea thermotolerans CBS 6340]
>gi|255721795|ref|XP_002545832.1| heat shock protein 60, mitochondrial precursor [Candida
tropicalis MYA-3404] >gi|240136321|gb|EER35874.1| heat shock protein 60, mitochondrial
precursor [Candida tropicalis MYA-3404]
>gi|255941288|ref|XP_002561413.1| Pc16g11070 [Penicillium chrysogenum Wisconsin 54-
1255] >gi|211586036|emb|CAP93777.1| Pc16g11070 [Penicillium chrysogenum Wisconsin 54-
1255]
>gi|259148241|emb|CAY81488.1| Hsp60p [Saccharomyces cerevisiae EC1118]
>gi|260950325|ref|XP_002619459.1| heat shock protein 60, mitochondrial precursor
[Clavispora lusitaniae ATCC 42720] >gi|238847031|gb|EEQ36495.1| heat shock protein 60,
mitochondrial precursor [Clavispora lusitaniae ATCC 42720]
>gi|261194577|ref|XP_002623693.1| chaperonin GroL [Ajellomyces dermatitidis SLH14081]
>gi|239588231|gb|EEQ70874.1| chaperonin GroL [Ajellomyces dermatitidis SLH14081]
>gi|327355067|gb|EGE83924.1| chaperonin GroL [Ajellomyces dermatitidis ATCC 18188]
>gi|296422271|ref|XP_002840685.1| hypothetical protein [Tuber melanosporum Mel28]
>gi|295636906|emb|CAZ84876.1| unnamed protein product [Tuber melanosporum]
>gi|296809035|ref|XP_002844856.1| heat shock protein 60 [Arthroderma otae CBS 113480]
>gi|238844339|gb|EEQ34001.1| heat shock protein 60 [Arthroderma otae CBS 113480]
>gi|302308696|ref|NP_985702.2| AFR155Wp [Ashbya gossypii ATCC 10895]
>gi|299790751|gb|AAS53526.2| AFR155Wp [Ashbya gossypii ATCC 10895]
>gi|374108933|gb|AEY97839.1| FAFR155Wp [Ashbya gossypii FDAG1]
>gi|302412525|ref|XP_003004095.1| heat shock protein [Verticillium albo-atrum VaMs. 102]
>gi|261356671|gb|EEY19099.1| heat shock protein [Verticillium albo-atrum VaMs. 102]
>gi|302505585|ref|XP_003014499.1| hypothetical protein ARB_07061 [Arthroderma
benhamiae CBS 112371] >gi|291178320|gb|EFE34110.1| hypothetical protein ARB_07061
[Arthroderma benhamiae CBS 112371]
>gi|302656385|ref|XP_003019946.1| hypothetical protein TRV_05992 [Trichophyton
verrucosum HKI 0517] >gi|291183723|gb|EFE39322.1| hypothetical protein TRV_05992
[Trichophyton verrucosum HKI 0517]
>gi|302915513|ref|XP_003051567.1| predicted protein [Nectria haematococca mpVI 77-13-4]
>gi|256732506|gb|EEU45854.1| predicted protein [Nectria haematococca mpVI 77-13-4]
>gi|310794550|gb|EFQ30011.1| chaperonin GroL [Glomerella graminicola M1.001]
>gi|315048491|ref|XP_003173620.1| chaperonin GroL [Arthroderma gypseum CBS 118893]
>gi|311341587|gb|EFR00790.1| chaperonin GroL [Arthroderma gypseum CBS 118893]
>gi|320580028|gb|EFW94251.1| Tetradecameric mitochondrial chaperonin [Ogataea
parapolymorpha DL-1]
>gi|320586014|gb|EFW98693.1| heat shock protein mitochondrial precursor [Grosmannia
clavigera kw1407]
>gi|322692465|gb|EFY84374.1| Heat shock protein 60 precursor (Antigen HIS-62)
[Metarhizium acridum CQMa 102]
>gi|322705285|gb|EFY96872.1| Heat shock protein 60 (Antigen HIS-62) [Metarhizium
anisopliae ARSEF 23]
>gi|323303806|gb|EGA57589.1| Hsp60p [Saccharomyces cerevisiae FostersB]
>gi|323307999|gb|EGA61254.1| Hsp60p [Saccharomyces cerevisiae FostersO]
>gi|323332364|gb|EGA73773.1| Hsp60p [Saccharomyces cerevisiae AWRI796]
>gi|326468648|gb|EGD92657.1| heat shock protein 60 [Trichophyton tonsurans CBS 112818]
>gi|326479866|gb|EGE03876.1| chaperonin GroL [Trichophyton equinum CBS 127.97]
>gi|330915493|ref|XP_003297052.1| hypothetical protein PTT_07333 [Pyrenophora teres f.
teres 0-1] >gi|311330479|gb|EFQ94847.1| hypothetical protein PTT_07333 [Pyrenophora teres
f. teres 0-1]
>gi|336271815|ref|XP_003350665.1| hypothetical protein SMAC_02337 [Sordaria macrospora
k-hell] >gi|380094827|emb|CCC07329.1| unnamed protein product [Sordaria macrospora k-
hell]
>gi|336468236|gb|EGO56399.1| hypothetical protein NEUTE1DRAFT_122948 [Neurospora
tetrasperma FGSC 2508]
>gi|340522598|gb|EGR52831.1| hsp60 mitochondrial precursor-like protein [Trichoderma
reesei QM6a]
>gi|341038907|gb|EGS23899.1| mitochondrial heat shock protein 60-like protein [Chaetomium
thermophilum var. thermophilum DSM 1495]
>gi|342886297|gb|EGU86166.1| hypothetical protein FOXB_03302 [Fusarium oxysporum
Fo5176]
>gi|344230084|gb|EGV61969.1| chaperonin GroL [Candida tenuis ATCC 10573]
>gi|344303739|gb|EGW33988.1| hypothetical protein SPAPADRAFT_59397 [Spathaspora
passalidarum NRRL Y-27907]
>gi|345560428|gb|EGX43553.1| hypothetical protein AOL_s00215g289 [Arthrobotrys
oligospora ATCC 24927]
>gi|346323592|gb|EGX93190.1| heat shock protein 60 (Antigen HIS-62) [Cordyceps militaris
CM01]
>gi|346975286|gb|EGY18738.1| heat shock protein [Verticillium dahliae VdLs.17]
>gi|354545932|emb|CCE42661.1| hypothetical protein CPAR2_203040 [Candida parapsilosis]
>gi|358369894|dbj|GAA86507.1| heat shock protein 60, mitochondrial precursor [Aspergillus
kawachii IFO 4308]
>gi|358386867|gb|EHK24462.1| hypothetical protein TRIVIDRAFT_79041 [Trichoderma virens
Gv29-8]
>gi|358399658|gb|EHK48995.1| hypothetical protein TRIATDRAFT_297734 [Trichoderma
atroviride IMI 206040]
>gi|363750488|ref|XP_003645461.1| hypothetical protein Ecym_3140 [Eremothecium
cymbalariae DBVPG#7215]
>gi|356889095|gb|AET38644.1| Hypothetical protein Ecym_3140 [Eremothecium cymbalariae
DBVPG#7215]
>gi|365759369|gb|EHN01160.1| Hsp60p [Saccharomyces cerevisiae × Saccharomyces
kudriavzevii VIN7]
>gi|365764091|gb|EHN05616.1| Hsp60p [Saccharomyces cerevisiae × Saccharomyces
kudriavzevii VIN7]
>gi|365985626|ref|XP_003669645.1| hypothetical protein NDAI_0D00880 [Naumovozyma
dairenensis CBS 421]
>gi|343768414|emb|CCD24402.1| hypothetical protein NDAI_0D00880 [Naumovozyma
dairenensis CBS 421]
>gi|366995970|ref|XP_003677748.1| hypothetical protein NCAS_0H00890 [Naumovozyma
castellii CBS 4309]
>gi|342303618|emb|CCC71399.1| hypothetical protein NCAS_0H00890 [Naumovozyma
castellii CBS 4309]
>gi|367005154|ref|XP_003687309.1| hypothetical protein TPHA_0J00520 [Tetrapisispora
phaffii CBS 4417] >gi|357525613|emb|CCE64875.1| hypothetical protein TPHA_0J00520
[Tetrapisispora phaffii CBS 4417]
>gi|367017005|ref|XP_003683001.1| hypothetical protein TDEL_0G04230 [Torulaspora
delbrueckii] >gi|359750664|emb|CCE93790.1| hypothetical protein TDEL_0G04230
[Torulaspora delbrueckii]
>gi|367035486|ref|XP_003667025.1| hypothetical protein MYCTH_2097570 [Myceliophthora
thermophila ATCC 42464]
>gi|347014298|gb|AEO61780.1| hypothetical protein MYCTH_2097570 [Myceliophthora
thermophila ATCC 42464]
>gi|367055018|ref|XP_003657887.1| hypothetical protein THITE_127923 [Thielavia terrestris
NRRL 8126] >gi|347005153|gb|AEO71551.1| hypothetical protein THITE_127923 [Thielavia
terrestris NRRL 8126]
>gi|378728414|gb|EHY54873.1| heat shock protein 60 [Exophiala dermatitidis NIH/UT8656]
>gi|380494593|emb|CCF33032.1| heat shock protein 60 [Colletotrichum higginsianum]
>gi|385305893|gb|EIF49836.1| heat shock protein 60 [Dekkera bruxellensis AWRI1499]
>gi|389638386|ref|XP_003716826.1| heat shock protein 60 [Magnaporthe oryzae 70-15]
>gi|351642645|gb|EHA50507.1| heat shock protein 60 [Magnaporthe oryzae 70-15]
>gi|440474658|gb|ELQ43388.1| heat shock protein 60 [Magnaporthe oryzae Y34]
>gi|440480475|gb|ELQ61135.1| heat shock protein 60 [Magnaporthe oryzae P131]
>gi|393243142|gb|EJD50658.1| chaperonin GroL [Auricularia delicata TFB-10046 SS5]
>gi|396494741|ref|XP_003844378.1| similar to heat shock protein 60 [Leptosphaeria maculans
JN3] >gi|312220958|emb|CBY00899.1| similar to heat shock protein 60 [Leptosphaeria
maculans JN3]
>gi|398393428|ref|XP_003850173.1| chaperone ATPase HSP60 [Zymoseptoria tritici IPO323]
>gi|339470051|gb|EGP85149.1| hypothetical protein MYCGRDRAFT_75170 [Zymoseptoria
tritici IPO323]
>gi|401624479|gb|EJS42535.1| hsp60p [Saccharomyces arboricola H-6]
>gi|401842294|gb|EJT44530.1| HSP60-like protein [Saccharomyces kudriavzevii IFO 1802]
>gi|402076594|gb|EJT72017.1| heat shock protein 60 [Gaeumannomyces graminis var. tritici
R3-111a-1]
>gi|403213867|emb|CCK68369.1| hypothetical protein KNAG_0A07160 [Kazachstania
naganishii CBS 8797]
>gi|406606041|emb|CCH42514.1| Heat shock protein 60, mitochondrial [Wickerhamomyces
ciferrii]
>gi|406863285|gb|EKD16333.1| heat shock protein 60 [Marssonina brunnea f. sp.
‘multigermtubi’ MB_m1]
>gi|407922985|gb|EKG16075.1| Chaperonin Cpn60 [Macrophomina phaseolina MS6]
>gi|408399723|gb|EKJ78816.1| hypothetical protein FPSE_00959 [Fusarium
pseudograminearum CS3096]
>gi|410083028|ref|XP_003959092.1| hypothetical protein KAFR_0I01760 [Kazachstania
africana CBS 2517] >gi|372465682|emb|CCF59957.1| hypothetical protein KAFR_0I01760
[Kazachstania africana CBS 2517]
>gi|444315528|ref|XP_004178421.1| hypothetical protein TBLA_0B00580 [Tetrapisispora
blattae CBS 6284] >gi|387511461|emb|CCH58902.1| hypothetical protein TBLA_0B00580
[Tetrapisispora blattae CBS 6284]
>gi|448090588|ref|XP_004197110.1| Piso0_004347 [Millerozyma farinosa CBS 7064]
>gi|448095015|ref|XP_004198141.1| Piso0_004347 [Millerozyma farinosa CBS 7064]
>gi|359378532|emb|CCE84791.1| Piso0_004347 [Millerozyma farinosa CBS 7064]
>gi|359379563|emb|CCE83760.1| Piso0_004347 [Millerozyma farinosa CBS 7064]
>gi|448526196|ref|XP_003869293.1| Hsp60 heat shock protein [Candida orthopsilosis Co 90-
125] >gi|380353646|emb|CCG23157.1| Hsp60 heat shock protein [Candida orthopsilosis]
>gi|46123737|ref|XP_386422.1| HS60_AJECA Heat shock protein 60, mitochondrial precursor
(Antigen HIS-62) [Gibberella zeae PH-1]
>gi|50292099|ref|XP_448482.1| hypothetical protein [Candida glabrata CBS 138]
>gi|49527794|emb|CAG61443.1| unnamed protein product [Candida glabrata]
>gi|50310975|ref|XP_455510.1| hypothetical protein [Kluyveromyces lactis NRRL Y-1140]
>gi|49644646|emb|CAG98218.1| KLLA0F09449p [Kluyveromyces lactis]
>gi|50422027|ref|XP_459575.1| DEHA2E05808p [Debaryomyces hansenii CBS767]
>gi|49655243|emb|CAG87802.1| DEHA2E05808p [Debaryomyces hansenii CBS767]
>gi|50555023|ref|XP_504920.1| YALI0F02805p [Yarrowia lipolytica]
>gi|49650790|emb|CAG77725.1| YALI0F02805p [Yarrowia lipolytica CLIB122]
>gi|6323288|ref|NP_013360.1| Hsp60p [Saccharomyces cerevisiae S288c]
>gi|123579|sp|P19882.1|HSP60_YEAST RecName: Full = Heat shock protein 60, mitochondrial;
AltName: Full = CPN60; AltName: Full = P66; AltName: Full = Stimulator factor I 66 kDa
component; Flags: Precursor >gi|171720|gb|AAA34690.1| heat shock protein 60 (HSP60)
[Saccharomyces cerevisiae] >gi|577181|gb|AAB67380.1| Hsp60p: Heat shock protein 60
[Saccharomyces cerevisiae] >gi|151941093|gb|EDN59473.1| chaperonin [Saccharomyces
cerevisiae YJM789] >gi|190405319|gb|EDV08586.1| chaperonin [Saccharomyces cerevisiae
RM11-1a] >gi|207342889|gb|EDZ70518.1| YLR259Cp-like protein [Saccharomyces cerevisiae
AWRI1631] >gi|256271752|gb|EEU06789.1| Hsp60p [Saccharomyces cerevisiae JAY291]
>gi|285813676|tpg|DAA09572.1| TPA: chaperone ATPase HSP60 [Saccharomyces cerevisiae
S288c] >gi|323353818|gb|EGA85673.1| Hsp60p [Saccharomyces cerevisiae VL3]
>gi|349579966|dbj|GAA25127.1| K7_Hsp60p [Saccharomyces cerevisiae Kyokai no. 7]
>gi|392297765|gb|EIW08864.1| Hsp60p [Saccharomyces cerevisiae CEN.PK113-7D]
>gi|226279|prf∥1504305A mitochondrial assembly factor
>gi|68485963|ref|XP_713100.1| heat shock protein 60 [Candida albicans SC5314]
>gi|68486010|ref|XP_713077.1| heat shock protein 60 [Candida albicans SC5314]
>gi|6016258|sp|O74261.1|HSP60_CANAL RecName: Full = Heat shock protein 60,
mitochondrial; AltName: Full = 60 kDa chaperonin; AltName: Full = Protein Cpn60; Flags:
Precursor >gi|3552009|gb|AAC34885.1| heat shock protein 60 [Candida albicans]
>gi|46434552|gb|EAK93958.1| heat shock protein 60 [Candida albicans SC5314]
>gi|46434577|gb|EAK93982.1| heat shock protein 60 [Candida albicans SC5314]
>gi|71001164|ref|XP_755263.1| antigenic mitochondrial protein HSP60 [Aspergillus fumigatus
Af293] >gi|66852901|gb|EAL93225.1| antigenic mitochondrial protein HSP60, putative
[Aspergillus fumigatus Af293] >gi|159129345|gb|EDP54459.1| antigenic mitochondrial protein
HSP60, putative [Aspergillus fumigatus A1163]
>gi|90970323|gb|ABE02805.1| heat shock protein 60 [Rhizophagus intraradices]

The recombinant yeast cell preferably comprises, respectively functionally expresses, a GroES chaperone and a GroEL chaperone. Preferably a 10 kDa chaperone (“GroES”) from Table 4 is combined with a matching 60 kDa chaperone (“GroEL”) from Table 5 of the same organism genus or species for expression in the recombinant yeast cell.

For instance: >gi|189189366|ref|XP_001931022.1|:71-168 10 kDa chaperonin [Pyrenophora tritici-repentis]expressed together with matching >gi|189190432|ref|XP_001931555.1|heat shock protein 60, mitochondrial precursor [Pyrenophora tritici-repentis Pt-1C-BFP]. All other combinations from Table 4 and 5 similarly made with same organism source are also available to the skilled person for expression. Furthermore, one may combine a chaperone from Table 4 from one organism with a chaperone from Table 5 from another organism, or one may combine GroES with a chaperone from Table 5, or one may combine GroEL with a chaperone from Table 4.

Preferably the molecular chaperone(s) thus comprise or consist of:
an amino acid sequence of SEQ ID NO: 5 and/or SEQ ID NO:7; or
one or more functional homologue(s) of SEQ ID NO: 5 and/or SEQ ID NO:7, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of respectively SEQ ID NO: 5 and/or SEQ ID NO:7; or
one or more functional homologue(s) of SEQ ID NO: 5 and/or SEQ ID NO:7, having one or more mutations, substitutions, insertions and/or deletions when compared with the amino acid sequence of respectively SEQ ID NO: 5 and/or SEQ ID NO:7, more preferably one or more functional homologue(s) that has/have no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions when compared with the amino acid sequence of respectively SEQ ID NO: 5 and/or SEQ ID NO:7.

Preferable the nucleic acid sequence(s) encoding the molecular chaperones comprise or consist of:
a nucleic acid sequence of SEQ ID NO: 6 and/or SEQ ID NO: 8; or
one or more functional homologue(s) of SEQ ID NO: 6 and/or SEQ ID NO: 8, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of respectively SEQ ID NO: 6 and/or SEQ ID NO: 8; or
one or more functional homologue(s) of SEQ ID NO: 6 and/or SEQ ID NO: 8, having one or more mutations, substitutions, insertions and/or deletions when compared with the nucleic acid sequence of respectively SEQ ID NO: 6 and/or SEQ ID NO: 8, more preferably one or more functional homologue(s) of that has/have no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions when compared with the nucleic acid sequence of respectively SEQ ID NO: 6 and/or SEQ ID NO: 8.

The nucleic acid sequence(s) encoding for the molecular chaperones may suitably be incorporated in the genome of the recombinant yeast cell, for example as described in the examples of WO2014/129898, herein incorporated by reference.

Deletion or Disruption of Glycerol 3-Phosphate Phosphohydrolase and/or Glycerol 3-Phosphate Dehydrogenase

The recombinant yeast cell further may or may not comprise a deletion or disruption of one or more endogenous nucleotide sequence encoding a glycerol 3-phosphate phosphohydrolase gene and/or encoding a glycerol 3-phosphate dehydrogenase gene.

Preferably enzymatic activity needed for the NADH-dependent glycerol synthesis in the yeast cell is reduced or deleted. The reduction or deletion of the enzymatic activity of glycerol 3-phosphate phosphohydrolase and/or glycerol 3-phosphate dehydrogenase can be achieved by modifying one or more genes encoding a NAD-dependent glycerol 3-phosphate dehydrogenase (GPD) and/or one or more genes encoding a glycerol phosphate phosphatase (GPP), such that the enzyme is expressed considerably less than in the wild-type or such that the gene encodes a polypeptide with reduced activity. Such modifications can be carried out using commonly known biotechnological techniques, and may in particular include one or more knock-out mutations or site-directed mutagenesis of promoter regions or coding regions of the structural genes encoding GPD and/or GPP. Alternatively, yeast strains that are defective in glycerol production may be obtained by random mutagenesis followed by selection of strains with reduced or absent activity of GPD and/or GPP. S. cerevisiae GPD1, GPD2, GPP1 and GPP2 genes are shown in WO2011010923, and are disclosed in SEQ ID NO: 24-27 of that application.

Preferably the recombinant yeast is a recombinant yeast that further comprises a deletion or disruption of a glycerol-3-phosphate dehydrogenase (GPD) gene. The one or more of the glycerol phosphate phosphatase (GPP) genes may or may not be deleted or disrupted.

More preferably the recombinant yeast is a recombinant yeast that comprises a deletion or disruption of a glycerol-3-phosphate dehydrogenase 1 (GPD1) gene. The glycerol-3-phosphate dehydrogenase 2 (GPD2) gene may or may not be deleted or disrupted.

Most preferably the recombinant yeast is a recombinant yeast that comprises a deletion or disruption of a glycerol-3-phosphate dehydrogenase 1 (GPD1) gene, whilst the glycerol-3-phosphate dehydrogenase 2 (GPD2) gene and/or the glycerol phosphate phosphatase (GPP) genes remain(s) active and/or intact. Preferably therefore, only one of the S. cerevisiae GPD1, GPD2, GPP1 and GPP2 genes is disrupted and deleted, whereas most preferably only GPD1 is chosen from the group consisting of GPD1, GPD2, GPP1 and GPP2 genes to be disrupted or deleted.

Without wishing to be bound to any kind of theory it is believed that a recombinant yeast according to the invention wherein the GPD1 gene, but not the GPD2 gene, is deleted or disrupted, can be advantageous when applied in a fermentation process wherein the fermentation medium comprises, at least during part of the process, a concentration of glucose that is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L.

Preferably at least one gene encoding a GPD and/or at least one gene encoding a GPP is entirely deleted, or at least a part of the gene is deleted that encodes a part of the enzyme that is essential for its activity. Good results can be achieved with a S. cerevisiae cell, wherein the open reading frames of the GPD1 gene and/or of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. Suitably, good results can be been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that is designed to be deleted, for example by a diagnostic polymerase chain reaction or Southern hybridization.

Thus, in the recombinant yeast cells of the invention, glycerol 3-phosphate phosphohydrolase activity in the cell and/or glycerol 3-phosphate dehydrogenase activity in the cell can be advantageously reduced.

### Glycerol Re-Uptake

The recombinant yeast cell may or may not further comprise one or more additional nucleic acid sequences that are part of a glycerol re-uptake pathway. That is, the recombinant yeast cell may or may not further comprise:
one or more heterologous nucleic acid sequences encoding for a glycerol dehydrogenase; and/or
one or more homologous or heterologous nucleic acid sequences encoding for a dihydroxyacetone kinase; and/or
one or more heterologous nucleic acid sequences encoding for a glycerol transporter.

Thus, in one preferred embodiment the recombinant yeast cell is a recombinant yeast cell functionally expressing:
one or more heterologous nucleic acid sequences encoding for a ribulose-1,5-phosphate carboxylase/oxygenase (EC4.1.1.39; Rubisco), and optionally one or more nucleic acid sequences encoding for molecular chaperones for Rubisco;
one or more heterologous nucleic acid sequences encoding for phosphoribulokinase (EC2.7.1.19; PRK);
one or more nucleic acid sequences encoding for a transketolase (EC 2.2.1.1), wherein the transketolase is under control of a promoter (the “TKL promoter”) which has a TKL expression ratio _{anaerobic/aerobic} of 2 or more;
one or more heterologous nucleic acid sequences encoding for a glycerol dehydrogenase;
one or more homologous or heterologous nucleic acid sequences encoding for a dihydroxyacetone kinase; and
optionally one or more heterologous nucleic acid sequences encoding for a glycerol transporter.

Without wishing to be bound by any kind of theory it is believed that a recombinant yeast cell that further comprises a combination of glycerol dehydrogenase, dihydroxyacetone kinase and optionally a glycerol transporter has an improved overall performance in the form of higher ethanol yields.

In an alternative preferred embodiment the recombinant yeast cell is a recombinant yeast cell that does not functionally express:
one or more heterologous nucleic acid sequences encoding for a glycerol dehydrogenase; and/or
one or more heterologous nucleic acid sequences encoding for a dihydroxyacetone kinase; and/or
one or more heterologous nucleic acid sequences encoding for a glycerol transporter.

Without wishing to be bound by any kind of theory it is believed that in the absence of one or more of these features of such a glycerol re-uptake pathway, a recombinant yeast cell is obtained that has a very low accumulation of glucose and/or other sugars and has an improved robustness when applied in a medium comprising a high amount of sugars. The application of a recombinant yeast cell that does not comprise one or more of a, heterologous and/or homologous, glycerol dehydrogenase; heterologous and/or homologous dihydroxyacetone kinase and/or heterologous and/or homologous glycerol transporter can therefore be advantageous when applied in a fermentation process where the glucose at the start of or during the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L.

Most preferably, the recombinant yeast is therefore a recombinant yeast that is functionally expressing:
one or more heterologous nucleic acid sequences encoding for a ribulose-1,5-phosphate carboxylase/oxygenase (EC4.1.1.39; Rubisco), and optionally one or more nucleic acid sequences encoding for molecular chaperones for Rubisco;
one or more heterologous nucleic acid sequences encoding for phosphoribulokinase (EC2.7.1.19; PRK);
one or more nucleic acid sequences encoding for a transketolase (EC 2.2.1.1), wherein the transketolase is under control of a promoter (the “TKL promoter”) which has a TKL expression ratio _{anaerobic/aerobic} of 2 or more;
   wherein the recombinant yeast cell does not functionally express
one or more heterologous nucleic acid sequences encoding for a glycerol dehydrogenase; and/or
one or more heterologous nucleic acid sequences encoding for a dihydroxyacetone kinase; and/or
one or more heterologous nucleic acid sequences encoding for a glycerol transporter.

### Glycerol Dehydrogenase

As indicated above, the recombinant yeast cell may or may not functionally express
a nucleic acid sequence encoding for a protein having glycerol dehydrogenase activity (E.C. 1.1.1.6);
a nucleic acid sequence encoding a protein having dihydroxyacetone kinase activity (E.C. 2.7.1.28 or E.C. 2.7.1.29); and
optionally a nucleic acid sequence encoding a protein having glycerol transporter activity.

Thus the recombinant yeast cell may or may not functionally express one or more, preferably heterologous, nucleic acid sequences encoding for a glycerol dehydrogenase.

If a glycerol dehydrogenase is present, the recombinant yeast cell may comprise a NAD⁺ linked glycerol dehydrogenase (EC 1.1.1.6) and/or a NADP⁺ linked glycerol dehydrogenase (EC 1.1.1.72). That is, the recombinant yeast cell may or may not comprise a nucleic acid sequence encoding a protein having NAD⁺ dependent glycerol dehydrogenase activity (EC 1.1.1.6) and/or a nucleic acid sequence encoding a protein having NADP⁺ dependent glycerol dehydrogenase activity (EC 1.1.1.72).

In one embodiment the protein having glycerol dehydrogenase activity is preferably a protein having NAD+ dependent glycerol dehydrogenase activity (EC 1.1.1.6) and preferably the recombinant yeast cell functionally expresses a nucleic acid sequence encoding a protein having NAD⁺ dependent glycerol dehydrogenase activity (EC 1.1.1.6). Such protein may be from bacterial origin or for instance from fungal origin. An example is gldA from E. coli.

In an alternative or additional embodiment, a NADP⁺ dependent glycerol dehydrogenase can be present (EC 1.1.1.72).

If a glycerol dehydrogenase is present, a NAD⁺ linked glycerol dehydrogenase is preferred.

A protein having glycerol dehydrogenase activity is herein also referred to as “glycerol dehydrogenase protein”, “glycerol dehydrogenase enzyme” or simply as “glycerol dehydrogenase”. In analogy thereto a protein having NAD+ dependent glycerol dehydrogenase activity is herein also referred to as “NAD+ dependent glycerol dehydrogenase protein”, “NAD+ dependent glycerol dehydrogenase enzyme” or simply as “NAD+ dependent glycerol dehydrogenase”. The glycerol dehydrogenase is abbreviated as GLD.

Preferences for a glycerol dehydrogenase and the nucleic sequences encoding for such are as described in WO2015028582, incorporated herein by reference.

NAD+ dependent glycerol dehydrogenase (EC 1.1.1.6) is an enzyme that catalyzes the chemical reaction:

glycerol+NAD⁺ [symbol] glycerone+NADH+H⁺

Thus, the two substrates of this enzyme are glycerol and NAD⁺, whereas its three products are glycerone, NADH, and H⁺. Glyceron and dihydroxyacetone are herein synonyms.

The glycerol dehydrogenase enzyme belongs to the family of oxidoreductases, specifically those acting on the CH—OH group of donor with NAD⁺ or NADP⁺ as acceptor. The systematic name of this enzyme class is glycerol:NAD⁺ 2-oxidoreductase. Other names in common use include glycerin dehydrogenase, and NAD⁺-linked glycerol dehydrogenase. This enzyme participates in glycerolipid metabolism. A glycerol dehydrogenase protein may be further defined by its amino acid sequence. Likewise a glycerol dehydrogenase protein may be further defined by a nucleotide sequence encoding the glycerol dehydrogenase protein. As explained in detail above under definitions, a certain glycerol dehydrogenase protein that is defined by a nucleotide sequence encoding the enzyme, includes (unless otherwise limited) the nucleotide sequence hybridising to such nucleotide sequence encoding the glycerol dehydrogenase protein.

The nucleic acid sequence encoding the protein having glycerol dehydrogenase activity can be a heterologous nucleic acid sequence. The protein having glycerol dehydrogenase activity can be a heterologous protein having NAD+ dependent glycerol dehydrogenase activity.

If the recombinant yeast cell comprises one or more heterologous nucleic acid sequences encoding for a glycerol dehydrogenase, the recombinant yeast cell preferably further comprises suitable co-factors to enhance the activity of the glycerol dehydrogenase. For example, the recombinant yeast cell may comprise zinc, zinc ions or zinc salts and/or one or more pathways to include such in the cell.

Suitable examples of heterologous proteins having glycerol dehydrogenase activity include the glycerol dehydrogenase proteins of respectively Klebsiella pneumoniae, Enterococcus aerogenes, Yersinia aldovae, and Escherichia coli. Their amino acid sequences of such proteins have been illustrated respectively by SEQ ID NO: 33, SEQ ID NO: 34, SEQ ID NO: 35 and SEQ ID NO: 36.

The recombinant yeast cell therefore may or may not include one or more, suitably heterologous, glycerol dehydrogenase proteins having an amino acid sequence of SEQ ID NO: 33, SEQ ID NO: 34, SEQ ID NO: 35 and/or SEQ ID NO: 36; and/or functional homologues thereof comprising an amino acid sequence having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 33, SEQ ID NO: 34, SEQ ID NO: 35 and/or SEQ ID NO: 36; and/or functional homologues thereof comprising an amino acid sequence having one or more mutations, substitutions, insertions and/or deletions as compared to the amino acid sequence of SEQ ID NO: 33, SEQ ID NO: 34, SEQ ID NO: 35 and/or SEQ ID NO: 36, wherein more preferably the amino acid sequence of such functional homologues has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions as compared to the amino acid sequence of SEQ ID NO: 33, SEQ ID NO: 34, SEQ ID NO: 35 and/or SEQ ID NO: 36.

A preferred glycerol dehydrogenase protein is the glycerol dehydrogenase protein encoded by the gldA gene from E. coli. SEQ ID NO: 36 shows the amino acid sequence of this preferred NAD+ dependent glycerol dehydrogenase protein, encoded by the gldA gene from E. coli. The nucleic acid sequence of the gldA gene of E. coli is illustrated by SEQ ID NO: 37.

If the recombinant yeast cell comprises one or more heterologous nucleic acid sequences encoding for a glycerol dehydrogenase, the recombinant yeast cell therefore most preferably comprises a heterologous nucleotide sequence encoding a protein having NAD+ dependent glycerol dehydrogenase activity (E.C. 1.1.1.6) derived from E. Coli, optionally codon-optimized for the host cell, as exemplified by the nucleic acid sequence shown in SEQ ID NO:37.

Preferable the nucleic acid sequence encoding the protein having glycerol dehydrogenase activity thus comprises or consists of:
a nucleic acid sequence of SEQ ID NO: 37; or
a functional homologue of SEQ ID NO: 37, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 37; or
a functional homologue of SEQ ID NO: 37, having one or more mutations, substitutions, insertions and/or deletions when compared with the nucleic acid sequence of SEQ ID NO:37, more preferably a functional homologue that has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions when compared with the nucleic acid sequence of SEQ ID NO: 37.

If the recombinant yeast cell comprises one or more heterologous nucleic acid sequences encoding for a glycerol dehydrogenase, the recombinant yeast cell therefore most preferably comprises one or more nucleotide sequence encoding a glycerol dehydrogenase (E.C. 1.1.1.6) derived from E. Coli, optionally codon-optimized for the host cell. Such heterologous nucleic acid sequence (e.g. the gene) encoding for the glycerol dehydrogenase protein may suitably be incorporated in the genome of the recombinant yeast cell, for example as described in the examples of WO2015/028583, herein incorporated by reference.

Further examples of suitable glycerol dehydrogenases are listed in Table 6(a) to 6(d). At the top of each table the gldA that is BLASTED is mentioned.

TABLE 6(a)
BLAST Query - gldA from Escherichia coli

| Description | Identity (%) | Accession number |
|---|---|---|
| glycerol dehydrogenase, NAD [Escherichia coli | 100 | NP_418380.4 |
| str. K-12 substr. MG1655] | | |
| glycerol dehydrogenase [Escherichia coli | 99 | YP_002331714.1 |
| O127:H6 str. E2348/69] | | |
| glycerol dehydrogenase [Citrobacter youngae] | 94 | WP_006686227.1 |
| glycerol dehydrogenase [Citrobacter freundii] | 92 | WP_003840533.1 |

TABLE 6(b)
BLAST Query - gldA from Klebsiella pneumoniae

| Description | Identity (%) | Accession number |
|---|---|---|
| glycerol dehydrogenase [Klebsiella pneumoniae 342] | 100 | YP_002236495.1 |
| glycerol dehydrogenase [Citrobacter freundii] | 93 | WP_003024745.1 |
| Glycerol dehydrogenase (EC 1.1.1.6) | 92 | YP_004590977.1 |
| [Enterobacter aerogenes EA1509E] | | |
| glycerol dehydrogenase [Escherichia coli] | 91 | WP_016241524.1 |
| glycerol dehydrogenase [Enterococcus aerogenes] | 87 | See examples |
| | | herein strains |
| | | with CAS15 |
| glycerol dehydrogenase [Yersinia aldovae] | 74 | WP_004701845.1 |
| glycerol dehydrogenase [Enterobacteriaceae bacterium | 61 | WP_017375113.1 |
| LSJC7] | | |
| glycerol dehydrogenase [Citrobacter youngae] | 60 | WP_006686227.1 |

TABLE 6(c)
BLAST Query - gldA from Enterococcus aerogenes

| Description | Identity (%) | Accession number |
|---|---|---|
| glycerol dehydrogenase [Enterobacter aerogenes | 100 | YP_004591726.1 |
| KCTC 2190] | | |
| Glycerol dehydrogenase (EC 1.1.1.6) | 99 | YP_007390021.1 |
| [Enterobacter aerogenes EA1509E] | | |
| glycerol dehydrogenase [Klebsiella pneumoniae] | 92 | WP_004203683.1 |
| glycerol dehydrogenase [Escherichia coli] | 88 | WP_001322519.1 |
| | | See examples |
| | | herein strains |
| | | with CAS13 |
| glycerol dehydrogenase [Enterobacter cloacae | 87 | YP_003615506.1 |
| subsp. cloacae ATCC 13047] | | |

TABLE 6(d)
BLAST Query - gldA from Yersinia aldovae

| Description | Identity (%) | Accession number |
|---|---|---|
| glycerol dehydrogenase [Yersinia aldovae] | 100 | WP_004701845.1 |
| glycerol dehydrogenase [Yersinia intermedia] | 95 | WP_005189747.1 |
| glycerol dehydrogenase [Serratia liquefaciens | 81 | YP_008232202.1 |
| ATCC 27592] | | |
| glycerol dehydrogenase [Escherichia coli] | 76 | WP_016241524.1 |
| | | See examples |
| | | herein strains |
| | | with CAS13. |
| hypothetical protein EAE_03845 [Enterobacter | 75 | YP_004590977.1 |
| aerogenes KCTC 2190] | | |
| glycerol dehydrogenase [Aeromonas hydrophila] | 65 | WP_017410769.1 |

### Dihydroxyacetone Kinase

As indicated above, the recombinant yeast cell may or may not functionally express
a nucleic acid sequence encoding for a protein having glycerol dehydrogenase activity (E.C. 1.1.1.6);
a nucleic acid sequence encoding a protein having dihydroxyacetone kinase activity (E.C. 2.7.1.28 or E.C. 2.7.1.29); and
optionally a nucleic acid sequence encoding a protein having glycerol transporter activity.

That is, the recombinant yeast cell may or may not functionally express one or more, homologous or heterologous, nucleic acid sequences encoding for dihydroxyacetone kinase (E.C. 2.7.1.28 or E.C. 2.7.1.29),

A protein having dihydroxyacetone kinase activity is herein also referred to as “dihydroxyacetone kinase protein”, “dihydroxyacetone kinase enzyme” or simply as “dihydroxyacetone kinase”. The dihydroxyacetone kinase is abbreviated herein as DAK.

Preferences for a dihydroxyacetone kinase and the nucleic sequences encoding for such are as described in WO2015028582, incorporated herein by reference.

The protein having dihydroxy kinase activity may suitably belong to the enzyme categories of E.C. 2.7.1.28 and/or E.C. 2.7.1.29. The recombinant yeast cell thus suitably functionally expresses a nucleic acid sequence encoding a protein having dihydroxyacetone kinase activity (E.C. 2.7.1.28 and/or E.C. 2.7.1.29).

A dihydroxyacetone kinase is preferably herein understood as an enzyme that catalyzes the chemical reaction (EC 2.7.1.29):

ATP+glycerone↔ADP+glycerone phosphate

and/or the chemical reaction (EC 2.7.1.28):

ATP+D-glyceraldehyde↔ADP+D-glyceraldehyde 3-phosphate.

Other names in common use for a dihydroxyacetone kinase include glycerone kinase, ATP:glycerone phosphotransferase and (phosphorylating) acetol kinase. It is further understood that glycerone and dihydroxyacetone are the same molecule. A dihydroxyacetone kinase protein may be further defined by its amino acid sequence. Likewise a dihydroxyacetone kinase protein may be further defined by a nucleotide sequence encoding the dihydroxyacetone kinase protein. As explained in detail above under definitions, a certain dihydroxyacetone kinase protein that is defined by a nucleotide sequence encoding the enzyme, includes (unless otherwise limited) the nucleotide sequence hybridising to such nucleotide sequence encoding the dihydroxyacetone kinase protein.

If present, the recombinant yeast cell preferably functionally expresses a nucleic acid sequence encoding a native protein having dihydroxyacetone kinase activity. More preferably, the nucleic acid sequence encoding the protein having dihydroxyacetone kinase activity is a native nucleic acid sequence.

Yeast comprises two native isozymes of dihydroxyacetone kinase (DAK1 and DAK2). These native dihydroxyacetone kinase enzymes are preferred according to the invention. Preferably the host cell is a Saccharomyces cerevisiae cell and preferably the above native dihydroxyacetone kinase enzymes are the native dihydroxyacetone kinase enzymes of a Saccharomyces cerevisiae yeast cell. The amino acid sequences of the native dihydroxyacetone kinase proteins of Saccharomyces cerevisiae, DAK1 and DAK2, have been illustrated respectively by SEQ ID NO: 38 and SEQ ID NO: 39. The nucleic acid sequences coding for these native dihydroxyacetone kinase proteins DAK1 and DAK2 have been illustrated respectively by SEQ ID NO: 43 and SEQ ID NO: 44.

It is also possible for the recombinant yeast cell to functionally express a nucleic acid sequence encoding a protein having dihydroxyacetone kinase activity, where the nucleic acid sequence is a heterologous nucleic acid sequence, respectively wherein the protein is a heterologous protein. In an embodiment the recombinant yeast cell comprises a heterologous gene encoding a dihydroxyacetone kinase. Suitable heterologous genes include the genes encoding dihydroxyacetone kinases from Saccharomyces kudriavzevii, Zygosaccharomyces bailii, Kluyveromyces lactis, Candida glabrata, Yarrowia lipolytica, Klebsiella pneumoniae, Enterobacter aerogenes, Escherichia coli, Yarrowia lipolytica, Schizosaccharomyces pombe, Botryotinia fuckeliana, and Exophiala dermatitidis. Preferred heterologous proteins having dihydroxyacetone kinase activity include those derived from respectively Klebsiella pneumoniae, Yarrowia lipolytica and Schizosaccharomyces pombe, as illustrated respectively by SEQ ID NO: 40, SEQ ID NO: 41 and SEQ ID NO: 42.

The recombinant yeast cell may or may not comprise a genetic modification that causes overexpression of a dihydroxyacetone kinase, for example by overexpression of a nucleic acid sequence encoding a protein having dihydroxyacetone kinase activity. The nucleotide sequence encoding the dihydroxyacetone kinase may be native or heterologous to the cell. Nucleic acid sequences that may be used for overexpression of dihydroxyacetone kinase in the cells of the invention are for example the dihydroxyacetone kinase genes from S. cerevisiae (DAK1) and (DAK2) as e.g. described by Molin et al., “Dihydroxy-acetone kinases in Saccharomyces cerevisiae are involved in detoxification of dihydroxyacetone” (2003), J. Biol. Chem., vol. 278: pages 1415-1423, incorporated herein by reference. In a preferred embodiment a codon-optimised (see above) nucleotide sequence encoding the dihydroxyacetone kinase is overexpressed, such as e.g. a codon optimised nucleotide sequence encoding the dihydroxyacetone kinase of SEQ ID NO: 38, SEQ ID NO: 39, SEQ ID NO: 40, SEQ ID NO: 41 or SEQ ID NO: 42.

As indicated above, the native nucleic acid sequences encoding dihydroxyacetone kinase proteins in Saccharomyces cerevisiae, DAK1 and DAK2, have been illustrated respectively by SEQ ID NO: 43 and SEQ ID NO: 44.

Preferably the recombinant yeast cell does comprise a genetic modification that increases the specific activity of any dihydroxyacetone kinase in the cell. For example, the recombinant yeast cell may comprise one or more native and/or heterologous nucleic acid sequence encoding one or more native and/or heterologous dihydroxyacetone kinase protein(s), such as DAK1 and/or DAK2, that is/are overexpressed. A native dihydroxyacetone kinase, such as DAK1 and/or DAK2, may for example be overexpressed via one or more genetic modifications resulting in more copies of the gene encoding for the dihydroxy acetone kinase than present in the non-genetically modified cell, and/or a non-native promoter may be applied.

Preferably the recombinant yeast cell is a recombinant yeast cell, wherein the expression of the nucleic acid sequence encoding the protein having dihydroxyacetone kinase activity is under control of a promoter. The promoter can for example be a promoter that is native to another gene in the host cell.

For overexpression of the nucleotide sequence encoding the dihydroxyacetone kinase, the nucleotide sequence (to be overexpressed) can be placed in an expression construct wherein it is operably linked to suitable expression regulatory regions/sequences to ensure overexpression of the dihydroxyacetone kinase enzyme upon transformation of the expression construct into the host cell of the invention (see above). Suitable promoters for (over)expression of the nucleotide sequence coding for the enzyme having dihydroxyacetone kinase activity include promoters that are preferably insensitive to catabolite (glucose) repression, that are active under anaerobic conditions and/or that preferably do not require xylose or arabinose for induction. Examples of such promoters are given above. A dihydroxyacetone kinase that is overexpressed, is preferably overexpressed by at least a factor 1.1, 1.2, 1.5, 2, 5, 10 or 20 as compared to a strain which is genetically identical except for the genetic modification causing the overexpression. Preferably, the dihydroxyacetone kinase is overexpressed under anaerobic conditions by at least a factor 1.1, 1.2, 1.5, 2, 5, 10 or 20 as compared to a strain which is genetically identical except for the genetic modification causing the overexpression. It is to be understood that these levels of overexpression may apply to the steady state level of the enzyme's activity (specific activity in the cell), the steady state level of the enzyme's protein as well as to the steady state level of the transcript coding for the enzyme in the cell. Overexpression of the nucleotide sequence in the host cell produces a specific dihydroxyacetone kinase activity of at least 0.002, 0.005, 0.01, 0.02 or 0.05 U min-1 (mg protein)-1, determined in cell extracts of the transformed host cells at 30° C. as described e.g. in the Examples of WO2013/081456.

A most preferred dihydroxyacetone kinase protein is the dihydroxyacetone kinase protein encoded by the Dak1 gene from Saccharomyces cerevisiae. SEQ ID NO: 38 shows the amino acid sequence of a suitable dihydroxyacetone kinase protein, encoded by the Dak1 gene from Saccharomyces cerevisiae. SEQ ID NO: 43 illustrates the nucleic acid sequence of the Dak1 gene itself.

If the recombinant yeast cell comprises one or more overexpressed nucleic acid sequences encoding for a dihydroxyacetone kinase, the recombinant yeast cell therefore most preferably comprises one or more overexpressed nucleotide sequence encoding a dihydroxyacetone kinase derived from Saccharomyces cerevisiae, as exemplified by the nucleic acid sequence shown in SEQ ID NO: 43.

Preferably the protein having dihydroxy acetone kinase activity thus comprises or consists of:
an amino acid sequence of SEQ ID NO: 38, SEQ ID NO: 39, SEQ ID NO: 40, SEQ ID NO: 41 or SEQ ID NO: 42; or
a functional homologue of SEQ ID NO: 38, SEQ ID NO: 39, SEQ ID NO: 40, SEQ ID NO: 41 or SEQ ID NO: 42, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 38, SEQ ID NO: 39, SEQ ID NO: 40, SEQ ID NO: 41 or SEQ ID NO: 42; or
a functional homologue of SEQ ID NO: 38, SEQ ID NO: 39, SEQ ID NO: 40, SEQ ID NO: 41 or SEQ ID NO: 42, having one or more mutations, substitutions, insertions and/or deletions when compared with the amino acid sequence of SEQ ID NO: 38, SEQ ID NO: 39, SEQ ID NO: 40, SEQ ID NO: 41 or SEQ ID NO: 42, more preferably a functional homologue that has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions when compared with the amino acid sequence of SEQ ID NO: 38, SEQ ID NO: 39, SEQ ID NO: 40, SEQ ID NO: 41 or SEQ ID NO: 42.
   The protein having an amino acid sequence of SEQ ID NO: 38 and functional homologues thereof are most preferred.

Preferable the nucleic acid sequence encoding the protein having dihydroxy acetone kinase activity comprises or consists of:
a nucleic acid sequence of SEQ ID NO: 43 or SEQ ID NO: 44; or
a functional homologue of SEQ ID NO: 43 or SEQ ID NO: 44, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the nucleic acid sequence of SEQ ID NO: 43 or SEQ ID NO: 44; or
a functional homologue of SEQ ID NO: 43 or SEQ ID NO: 44, having one or more mutations, substitutions, insertions and/or deletions when compared with the nucleic acid sequence of SEQ ID NO: 43 or SEQ ID NO: 44, more preferably a functional homologue that has no more than 300, no more than 250, no more than 200, no more than 150, no more than 100, no more than 75, no more than 50, no more than 40, no more than 30, no more than 20, no more than 10 or no more than 5 nucleic acid mutations, substitutions, insertions and/or deletions when compared with the nucleic acid sequence of SEQ ID NO: 43 or SEQ ID NO: 44.

The nucleic acid sequence (e.g. the gene) encoding for the dihydroxy acetone kinase protein may suitably be incorporated in the genome of the recombinant yeast cell.

Examples of suitable dihydroxyacetone kinases are listed in Table 7(a) to 7(d). At the top of each table the DAK's used in the examples and that is BLASTED is mentioned.

TABLE 7(a)
BLAST Query - DAK1 from Saccharomyces cerevisiae

| Description | Identity (%) | Accession number |
|---|---|---|
| Dak1p [Saccharomyces cerevisiae S288c] | 100 | NP_013641.1 |
| dihydroxyacetone kinase [Saccharomyces cerevisiae YJM789] | 99 | EDN64325.1 |
| DAK1-like protein [Saccharomyces kudriavzevii IFO 1802] | 95 | EJT44075.1 |
| ZYBAOS11-03576g1_1 [Zygosaccharomyces bailii CLIB 213] | 77 | CDF91470.1 |
| hypothetical protein [Kluyveromyces lactis NRRL Y-1140] | 70 | XP_451751.1 |
| hypothetical protein [Candida glabrata CBS 138] | 63 | XP_449263.1 |
| Dak2p [Saccharomyces cerevisiae S288c] | 44 | NP_116602.1 |
| DAK1 [Yarrowia lipolytica] | 41 | See examples |
| | | herein strains |
| | | with CAS23 |

TABLE 7(b)
BLAST Query - dhaK from Klebsiella pneumoniae

| | Identity | |
|---|---|---|
| Description | (%) | Accession number |
| dihydroxyacetone kinase subunit DhaK | 100 | YP_002236493.1 |
| [Klebsiella pneumoniae 342] | | |
| dihydroxyacetone kinase subunit K | 99 | WP_004149886.1 |
| [Klebsiella pneumoniae] | | |
| dihydroxyacetone kinase subunit K | 96 | WP_020077889.1 |
| [Enterobacter aerogenes] | | |
| dihydroxyacetone kinase subunit DhaK | 88 | YP_002407536.1 |
| [Escherichia coli IAI39] | | |
| dihydroxyacetone kinase, DhaK subunit | 87 | WP_001398949.1 |
| [Escherichia coli] | | |

TABLE 7(c)
BLAST Query - DAK1 from Yarrowia lipolytica

| Description | Identity (%) | Accession number |
|---|---|---|
| YALI0F09273p [Yarrowia lipolytica] | 100 | XP_505199.1 |
| dihydroxyacetone kinase [Schizosaccharomyces pombe] | 46 | AAC83220.1 |
| dihydroxyacetone kinase Dak1 [Schizosaccharomyces | 45 | NP_593241.1 |
| pombe 972h-] | | |
| dihydroxyacetone kinase [Saccharomyces cerevisiae | 44 | EDV12567.1 |
| RM11-1a] | | |
| Dak2p [Saccharomyces cerevisiae JAY291] | 44 | EEU04233.1 |
| BN860_19306g1_1 [Zygosaccharomyces bailii CLIB 213] | 44 | CDF87998.1 |
| Dak1p [Saccharomyces cerevisiae CEN.PK113-7D] | 42 | EIW08612.1 |
| | | See examples |
| | | herein strains |
| | | with CAS21 |

TABLE 7(d)
BLAST Query - DAK1 from Schizosaccharomyces pombe

| Description | Identity (%) | Accession number |
|---|---|---|
| dihydroxyacetone kinase Dak1 [Schizosaccharomyces | 100 | NP_593241.1 |
| pombe 972h-] | | |
| putative dihydroxyacetone kinase protein | 48 | EMR88164.1 |
| [Botryotinia fuckeliana BcDW1] | | |
| Dihydroxyacetone kinase 1 [Fusarium oxysporum | 48 | ENH64704.1 |
| f. sp. cubense race 1] | | |
| Dak1p [Saccharomyces cerevisiae CEN.PK113-7D] | 46 | EIW08612.1 |
| Dak2p [Saccharomyces cerevisiae JAY291] | 44 | EEU04233.1 |
| dihydroxyacetone kinase [Exophiala dermatitidis | 42 | EHY55064.1 |
| NIH/UT8656] | | |

### Glycerol Transporter

The recombinant yeast cell can optionally, i.e. may or may not, comprise a nucleotide sequence encoding a glycerol transporter. Such a glycerol transporter can allow any glycerol that is externally available in the medium (e.g. from the backset in corn mash) or secreted after internal cellular synthesis to be transported into the cell and converted to ethanol.

If a glycerol transporter is present, the recombinant yeast preferably comprises one or more nucleic acid sequences encoding a heterologous glycerol transporter represented by amino acid sequence SEQ ID NO: 45, SEQ ID NO: 46 or a functional homologue thereof having an amino acid sequence identity of at least 50%, preferably at least 60%, 70%, 75%, 80%, 85%, 90%, 95%, 98%, or 99% with the amino acid sequence of SEQ ID NO: 45 and/or SEQ ID NO: 46.

In an embodiment the recombinant yeast can further comprise a deletion or disruption of one or more endogenous nucleotide sequences encoding a glycerol exporter (e.g. FPS1).

### Glucoamylase

Preferably, the recombinant yeast cell further functionally expresses a nucleic acid sequence encoding for a glucoamylase (EC 3.2.1.20 or 3.2.1.3).

A protein having glucoamylase activity is herein also referred to as “glucoamylase enzyme”, “glucoamylase protein” or simply “glucoamylase”. Glucoamylase has herein been abbreviated as “GA”.

Glucoamylase, also referred to as amyloglucosidase, alpha-glucosidase, glucan 1,4-alpha glucosidase, maltase glucoamylase, and maltase-glucoamylase, catalyses at least the hydrolysis of terminal 1,4-linked alpha-D-glucose residues from non-reducing ends of amylose chains to release free D-glucose. A glucoamylase may be further defined by its amino acid sequence.

Likewise a glucoamylase may be further defined by a nucleotide sequence encoding the glucoamylase. As explained in detail above under definitions, a certain glucoamylase that is defined by a nucleotide sequence encoding the enzyme, includes (unless otherwise limited) the nucleotide sequence hybridising to such nucleotide sequence encoding the glucoamylase.

Preferably the protein having glucoamylase activity comprises or consists of:
an amino acid sequence of SEQ ID NO: 47, SEQ ID NO: 48 or SEQ ID NO: 49; or
a functional homologue of SEQ ID NO: 47, SEQ ID NO: 48 or SEQ ID NO: 49, having at least 40%, at least 45%, at least 50%, at least 55%, at least 60%, at least 65%, at least 70%, at least 75%, at least 80%, at least 85%, at least 90% at least 95%, at least 98% or at least 99% sequence identity with the amino acid sequence of SEQ ID NO: 47, SEQ ID NO: 48 or SEQ ID NO: 49; or
a functional homologue of SEQ ID NO: 47, SEQ ID NO: 48 or SEQ ID NO: 49, having one or more mutations, substitutions, insertions and/or deletions when compared with the amino acid sequence of SEQ ID NO: 47, SEQ ID NO: 48 or SEQ ID NO: 49, more preferably a functional homologue that has no more than 300, no more than 250, no more than 200, no more than 150, no more than 100, no more than 75, no more than 50, no more than 40, no more than 30, no more than 20, no more than 10 or no more than 5 amino acid mutations, substitutions, insertions and/or deletions when compared with the amino acid sequence of SEQ ID NO: 47, SEQ ID NO: 48 or SEQ ID NO: 49.

The polypeptide of SEQ ID NO: 47 encodes a “mature glucoamylase”, referring to the enzyme in its final form after translation and any post-translational modifications, such as N-terminal processing, C-terminal truncation, glycosylation, phosphorylation, etc.

In an embodiment the nucleotide sequence encodes a polypeptide having an amino acid sequence of SEQ ID NO: 48 or a variant thereof having an amino acid sequence identity of at least 50%, preferably at least 60%, 70%, 75%, 80%, 85%, 90%, 95, 98%, or 99% with the amino acid sequence of SEQ ID NO: 48. Amino acids 1-17 of the SEQ ID NO: 48 may encode for a native signal sequence.

In another embodiment the nucleotide sequence allowing the expression of a glucoamylase encodes a polypeptide having an amino acid sequence of SEQ ID NO: 49 or a variant thereof having an amino acid sequence identity of at least 50%, preferably at least 60%, 70%, 75%, 80%, 85%, 90%, 95, 98%, or 99% with the amino acid sequence of SEQ ID NO: 49. Amino acids 1-19 of the SEQ ID NO: 49 may encode for a signal sequence.

A signal sequence (also referred to as signal peptide, targeting signal, localization signal, localization sequence, transit peptide, leader sequence or leader peptide) can be present at the N-terminus of a polypeptide (here, the glucoamylase) where it signals that the polypeptide is to be excreted, for example outside the cell and into the media.

### Recombinant Expression

The recombinant yeast cell is a recombinant cell. That is to say, a recombinant yeast cell comprises, or is transformed with or is genetically modified with a nucleotide sequence that does not naturally occur in the cell in question. Techniques for the recombinant expression of enzymes in a cell, as well as for the additional genetic modifications of a recombinant yeast cell are well known to those skilled in the art. Typically such techniques involve transformation of a cell with nucleic acid construct comprising the relevant sequence. Such methods are, for example, known from standard handbooks, such as Sambrook and Russel (2001) “Molecular Cloning: A Laboratory Manual”, (3rd edition), published by Cold Spring Harbor Laboratory Press, or F. Ausubel et al., eds., “Current protocols in molecular biology”, Green Publishing and Wiley Interscience, New York (1987). Methods for transformation and genetic modification of fungal host cells are known from e.g. EP-A-0635574, WO98/46772, WO 99/60102, WO00/37671, WO90/14423, EP-A-0481008, EP-A-0635574 and U.S. Pat. No. 6,265,186.

### Fermentation Process

The invention further provides a process for the production of ethanol, comprising converting a carbon source, preferably a carbohydrate or another organic carbon source, using a recombinant yeast cell as described in this specification, thereby forming ethanol.

The feed for this fermentation process suitably comprises one or more fermentable carbon sources. The fermentable carbon source preferably comprises or is consisting of one or more fermentable carbohydrates. More preferably, the fermentable carbon source comprises one or more mono-saccharides, disaccharides and/or polysaccharides. For example, the fermentable carbon source may comprise one or more carbohydrates selected from the group consisting of glucose, fructose, sucrose, maltose, xylose, arabinose, galactose, mannose and trehalose. The fermentable carbon source, preferably comprising or consisting of one or more carbohydrates, may suitably be obtained from starch, cellulose, hemicellulose lignocellulose, and/or pectin. Suitably the fermentable carbon source may be in the form of a, preferably aqueous, slurry, suspension, or a liquid.

The concentration of fermentable carbohydrate, such as for example glucose, during fermentation is preferably equal to or more than 80 g/L. That is, the initial concentration of glucose at the start of the fermentation, is preferably equal to or more than 80 g/L, more preferably equal to or more than 90 g/L, even more preferably equal to or more than 100 g/L, still more preferably equal to or more than 110 g/L, yet even more preferably equal to or more than 120 g/L, equal to or more than 130 g/L, equal to or more than 140 g/L, equal to or more than 150 g/L, equal to or more than 160 g/L, equal to or more than 170 g/L, or equal to or more than 180 g/L. The start of the fermentation may be the moment when the fermentable fermentable carbohydrate is brought into contact with the recombinant cell of the invention.

The fermentable carbon source may be prepared by contacting starch, lignocellulose, and/or pectin with an enzyme composition, wherein one or more mono-saccharides, disaccharides and/or polysaccharides are produced, and wherein the produced mono-saccharides, disaccharides and/or polysaccharides are subsequently fermented to give a fermentation product.

Before enzymatic treatment, the lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes. Subsequently the pretreated material can be subjected to enzymatic hydrolysis to release sugars that may be fermented according to the invention. This may be executed with conventional methods, e.g. contacting with cellulases, for instance cellobiohydrolase(s), endoglucanase(s), beta-glucosidase(s) and optionally other enzymes, The conversion with the cellulases may be executed at ambient temperatures or at higher temperatures, at a reaction time to release sufficient amounts of sugar(s). The result of the enzymatic hydrolysis is hydrolysis product comprising C5/C6 sugars, herein designated as the sugar composition.

Preferably at least part of the process according to the invention, such as for example at least part of the aerobic propagation step and/or at least part of the anaerobic fermentation step as described below, is carried out in the presence of a saccharolytic enzyme. By a saccharolytic enzyme is herein understood an enzyme that is capable of breaking up a oligosaccharide or polysaccharide. Examples of saccharolytic enzymes include glucoamylases, endoglucanase(s), beta-glucosidase(s). More preferably at least part of the process according to the invention is carried out in the presence of a glucoamylase. Such a glucoamylase can be externally added or it can be produced in-situ by the recombinant yeast cell itself. Most preferably the recombinant yeast cell is a recombinant yeast cell further comprising a, preferably heterologous, nucleic acid sequence encoding for a glucoamylase, such as for example exemplified in WO 2019/063543, herein incorporated by reference.

In one embodiment the fermentable carbohydrate is, or is comprised by a biomass hydrolysate, such as a corn stover or corn fiber hydrolysate. Such biomass hydrolysate may in its turn comprise, or be derived from corn stover and/or corn fiber.

By a “hydrolysate” is herein understood a polysaccharide-comprising material (such as corn stover, corn starch, corn fiber, or lignocellulosic material, which polysaccharides have been depolymerized through the addition of water to form mono and oligosaccharide sugars. Hydrolysates may be produced by enzymatic or acid hydrolysis of the polysaccharide-containing material.

A biomass hydrolysate may be a lignocellulosic biomass hydrolysate. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill iste, urban wood iste, municipal iste, logging iste, forest thinnings, short-rotation woody crops, industrial iste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal istes, lawn clippings, cotton, seaweed, algae (including macroalgae and microalgae), trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid iste, iste paper, yard iste, herbaceous material, agricultural residues, forestry residues, municipal solid iste, iste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic iste material generated from an agricultural process, forestry wood iste, or a combination of any two or more thereof. Algae, such as macroalgae and microalgae have the advantage that they may comprise considerable amounts of sugar alcohols such as sorbitol and/or mannitol. Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. In addition, pectins and other pectic substances such as arabinans may make up considerably proportion of the dry mass of typically cell walls from non-woody plant tissues (about a quarter to half of dry mass may be pectins). Lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

The process for the production of ethanol may comprise an aerobic propagation step and an anaerobic fermentation step. More preferably the process according to the invention is a process comprising an aerobic propagation step wherein a recombinant yeast cell population is formed; and an anaerobic fermentation step wherein the carbon source is converted to ethanol by using the recombinant yeast cell population.

By propagation is herein understood a process of recombinant yeast cell growth that leads to increase of an initial recombinant yeast cell population. Main purpose of propagation is to increase the population of the recombinant yeast cell using the recombinant yeast cell's natural reproduction capabilities as living organisms. That is, propagation is directed to the production of biomass and is not directed to the production of ethanol. The conditions of propagation may include adequate carbon source, aeration, temperature and nutrient additions. Propagation is an aerobic process, thus the propagation tank must be properly aerated to maintain a certain level of dissolved oxygen. Adequate aeration is commonly achieved by air inductors installed on the piping going into the propagation tank that pull air into the propagation mix as the tank fills and during recirculation. The capacity for the propagation mix to retain dissolved oxygen is a function of the amount of air added and the consistency of the mix, which is why water is often added at a ratio of between 50:50 to 90:10 mash to water. “Thick” propagation mixes (80:20 mash-to-water ratio and higher) often require the addition of compressed air to make up for the lowered capacity for retaining dissolved oxygen. The amount of dissolved oxygen in the propagation mix is also a function of bubble size, so some ethanol plants add air through spargers that produce smaller bubbles compared to air inductors. Along with lower glucose, adequate aeration is important to promote aerobic respiration during propagation, making the environment during propagation different from the anaerobic environment during fermentation.

By an anaerobic fermentation process is herein understood a fermentation step run under anaerobic conditions.

The anaerobic fermentation is preferably run at a temperature that is optimal for the cell. Thus, for most recombinant yeast cells, the fermentation process is performed at a temperature which is less than about 50° C., less than about 42° C., or less than about 38° C. For recombinant yeast cell or filamentous fungal host cells, the fermentation process is preferably performed at a temperature which is lower than about 35, about 33, about 30 or about 28° C. and at a temperature which is higher than about 20, about 22, or about 25° C.

The ethanol yield, based on xylose and/or glucose, in the process according to the invention is preferably at least about 50, about 60, about 70, about 80, about 90, about 95 or about 98%. The ethanol yield is herein defined as a percentage of the theoretical maximum yield.

The process according to the invention, and the propagation step and/or fermentation step suitably comprised therein can be carried out in batch, fed-batch or continuous mode. A separate hydrolysis and fermentation (SHF) process or a simultaneous saccharification and fermentation (SSF) process may also be applied.

The recombinant yeast and process according to the invention advantageously allow for a more robust process. Advantageously the process, or any anaerobic fermentation during the process can be carried out in the presence of high concentrations of carbon source. The process, respectively any anaerobic fermentation step therein, is therefore preferably carried out in the presence of a glucose concentration of 25 g/L or more, 30 g/L or more, 35 g/L or more, 40 g/L or more, 45 g/L or more, 50 g/L or more, 55 g/L or more, 60 g/L or more, 65 g/L or more, 70 g/L or more, 75 g/L or more, 80 g/L or more, 85 g/L or more, 90 g/L or more, 95 g/L or more, 100 g/L or more, 110 g/L or more, 120 g/L or more or may for example be in the range of 25 g/L-250 g/L, 30 gl/L-200 g/L, 40 g/L-200 g/L, 50 g/L-200 g/L, 60 g/L-200 g/L, 70 g/L-200 g/L, 80 g/L-200 g/L, or 90 g/L-200 g/L.

For the recovery of the fermentation product existing technologies are used. For different fermentation products different recovery processes are appropriate. Existing methods of recovering ethanol from aqueous mixtures commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol. In an embodiment in addition to the recovery of fermentation product, the yeast may be recycled.

The invention thus also provides a process for the production of ethanol, comprising converting a carbon source, preferably a carbohydrate, using a recombinant yeast cell as described herein before.

Preferably this process is at least partly carried out in a medium comprising glucose in a glucose concentration of 25 g/L or more, 30 g/L or more, 35 g/L or more, 40 g/L or more, 45 g/L or more, 50 g/L or more, 55 g/L or more, 60 g/L or more, 65 g/L or more, 70 g/L or more, 75 g/L or more, 80 g/L or more, 85 g/L or more, 90 g/L or more, 95 g/L or more, 100 g/L or more, 110 g/L or more, or 120 g/L or more.

Preferably this process is at least partly carried out in the presence of a saccharolytic enzyme, such as a glucoamylase.

As indicated above, the process preferably comprises an aerobic propagation step wherein a recombinant yeast cell population is formed; and an anaerobic fermentation step wherein the carbon source is converted to ethanol by using the recombinant yeast cell population. More preferably the anaerobic fermentation step is at least partly carried out in a medium comprising glucose in a glucose concentration of 25 g/L or more, 30 g/L or more, 35 g/L or more, 40 g/L or more, 45 g/L or more, 50 g/L or more, 55 g/L or more, 60 g/L or more, 65 g/L or more, 70 g/L or more, 75 g/L or more, 80 g/L or more, 85 g/L or more, 90 g/L or more, 95 g/L or more, 100 g/L or more, 110 g/L or more, or 120 g/L or more. In addition, the anaerobic fermentation step is preferably at least partly carried out in the presence of a saccharolytic enzyme, such as glucoamylase.

All patent and literature references cited in the present specification are hereby incorporated by reference in their entirety.

The following examples are offered for illustrative purposes only, and are not intended to limit the scope of the present invention in any way.

### EXAMPLES

### General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### HPLC Analysis

HPLC analysis is typically conducted as described in “Determination of sugars, byproducts and degradation products in liquid fraction in process sample”; Laboratory Analytical Procedure (LAP, Issue date: Dec. 8, 2006; by A. Sluiter, B. Hames, R. Ruiz, C. Scarlata, J. Sluiter, and D. Templeton; Technical Report (NREL/TP-51042623); January 2008; National Renewable Energy Laboratory.

After fermentation, samples for HPLC analysis were separated from yeast biomass and insoluble components (corn mash) by passing the clear supernatant after centrifugation through a 0.2 μm pore size filter

### Starter Strains

Strains were prepared using Ethanol Red® as starting strain. Ethanol Red® is a commercial Saccharomyces cerevisiae strain, available from Lesaffre.

A strain construction approach that can be followed is described in WO2013/144257A1 and WO2015/028582, incorporated herein by reference.

Expression cassettes from various genes of interest can be recombined in vivo into a pathway at a specific locus upon transformation of this yeast (U.S. Pat. No. 9,738,890 B2). The promoter, ORF and terminator sequences are assembled into expression cassettes with Golden Gate technology, as described by Engler et al (2011) and ligated into Bsal-digested backbone vectors that decorated the expression cassettes with the connectors for the in vivo recombination step. The expression cassettes including connectors are amplified by PCR. In addition, a 5′- and a 3′-DNA fragment of the up- and downstream part of the integration locus was amplified using PCR and decorated by a connector sequence. Upon transformation of yeast cells with these DNA fragments, in vivo recombination and integration into the genome takes place at the desired location. CRISPR-Cas9 technology is used to make a unique double stranded break at the integration locus to target the pathway to this specific locus (DiCarlo et al., 2013, Nucleic Acids Res 41:4336-4343) and WO16110512 and US2019309268. The gRNA was expressed from a multi-copy yeast shuttling vector that contains a natMX marker which confers resistance to the yeast cells against the antibiotic substance nourseothricin (NTC). The backbone of this plasmid is based on pRS305 (Sikorski and Hieter, Genetics 1989, vol. 122, pp. 19-27), including a functional 2 micron ORI sequence. The Streptococcus pyogenes CRISPR-associated protein 9 (Cas9) was expressed from a pRS414 plasmid (Sikorski and Hieter, 1989) with kanMX marker which confers resistance to the yeast cells against the antibiotic substance geneticin (G418). The guide RNA and protospacer sequences were designed with a gRNA designer tool (https://www.atum.bio/eCommerce/cas9/input).

### Example 1: Construction of “Rubisco” Strain (Intermediate Strain IX1)

In the current example the starter strain was transformed with the cbbMgene encoding the single subunit of ribulose-1,5-biphosphate-carboxylase (RuBisCO) from Thiobacfflus denitrificans and the genes encoding chaperonins GroEL and GroES from E. coli to aid in the proper folding of the RuBisCO protein in the cytosol of S. cerevisiae.

An expression cassette with S. cerevisiae TDH3 promoter, cbbM gene and S. cerevisiae CYC1 terminators was obtained from plasmid pBTWW002, the expression cassettes with S. cerevisiae TEF1 promoter, groEL and S. cerevisiae ACT1 terminator was obtained from plasmid pUD232, and the expression cassettes with S. cerevisiae TPI1 promoter, groES gene and S. cerevisiae PG11 terminator was obtained from plasmid pUD233, as described by Guadalupe-Medina et al., Biotechnol, Biofuels, 2013, vol. 6, p. 125) and US2019309268.

These three cassettes were integrated in the INT1 locus between NTR1 (YOR071c) and GYP1 (YOR070c) located on chromosome XV of S. cerevisiae with CRISPR-Cas9 using the gRNA expression cassette (as described by Di Carlo et al., Nucleic Acids Res. 2013; pp. 1-8). The cassette was ordered as synthetic DNA cassette (gBLOCK) at Integrated DNA Technologies (Leuven, Belgium) as INT1 gBLOCK and homologous recombination with PCR fragment (5′-INT1) generated with primers BoZ-783 and DBC-18463 genomic DNA of strain CEN.PK113-7D as template and a PCR fragment (3′-INT1) generated with primers DBC-18464 and BoZ-788 using genomic DNA of strain CEN.PK113-7D as template as described in WO2018/114762.

Diagnostic PCR was performed to confirm the correct assembly and integration at the INT1 locus of the GroES-cbbM-GroEL cassettes. Cas9 and gRNA plasmids were removed with twice a o/n liquid culture with YEPhD, dilution plated on YEPhD plate afterwards for single colonies. Streaks of cells made of the single colonies of the YEPhD plate and re-streaked on YEPhD and YEPhD+G418 (400 mg/ml) and YEPhD+NTC (200 mg/ml). Plasmid free colonies selected which have no growth on the antibiotic plates.

The above example resulted in intermediate strain IX1 which contained two copies of the corresponding cassette (see Table 8 for the detailed genotype).

### Example 2: Construction of Reference “PRK-Rubisco” Strain (Reference Strain RX2)

The intermediate strain IX1, as obtained in example 1, was transformed with phosphoribulokinase (prk) from S. oleacera (as described by Guadalupe-Medina et al., Biotechnol, Biofuels, 2013, vol. 6, p. 125) and ribulose-1,5-biphosphate-carboxylase (RuBisCO) from Thiobacfflus denitrificans encoded by the cbbM gene. The prk gene was expressed by the S. cerevisiae ANB1 promoter as described in U.S. Ser. No. 10/689,670 (illustrated by SEQ ID NO: 31) and the S. cerevisiae EB01 terminator (Sc_ENO1t) (illustrated in SEQ ID NO: 56).

The cbbMgene was cloned between the S. cerevisiae ENO2 promoter (illustrated by SEQ ID NO: 57) and S. cerevisiae YOX1 terminator (illustrated by SEQ ID NO: 58).

Cassettes were assembled into a pathway “prk-cbbM” and integrated in the INT14.02 locus, a non-coding region between ORF YNL179c and RPS3 (YNL178W) on chromosome XIV from S. cerevisiae using CRISPR-Cas9 with the INT14.02 protospacer (illustrated by SEQ ID NO: 59) and the following two sequences for homologous integration: Sc_INT14.02_FLANK5 (illustrated by SEQ ID NO: 60) and Sc_INT14.02_FLANK3 (illustrated by SEQ ID NO: 61).

Diagnostic PCR was performed to confirm the correct assembly and integration at the INT14.02 locus of the prk-cbbM cassettes, and the gRNA and Cas9 plasmids were removed as described above. This resulted in reference strain RX2 which contained two copies of prk, groEL and groES and 4 copies of cbbM (see Table 8 for detailed genotypes).

### Example 3: Construction of New Strains NX3 and NX4

New strains NX3 and NX4 were constructed by transforming the reference strain RX2 obtained in example 2 as follows:

A DNA fragment was compiled comprising the S. cerevisiae ANB1 promoter (illustrated by SEQ ID NO: 31), Pichia pastoris TKL1 gene (illustrated by SEQ ID NO: 26) and the S. cerevisiae TDH1 terminator. The DNA fragment was named “fragmentA” (illustrated by SEQ ID NO: 50). An expression cassette was assembled using Golden Gate Cloning (as described for example by Engler et al., “Generation of Families of Construct Variants Using Golden Gate Shuffling”, (2011), published in chapter 11 of Chaofu Lu et al. (eds.), cDNA Libraries: Methods and Applications, Methods in Molecular Biology, vol. 729, pages 167-180, incorporated herein by reference) and integrated in the INT95 locus between SOD1 (YJR104C) and ADO1 (YJR105W) located on chromosome X of S. cerevisiae reference strain RX2 using CRISPR-Cas9 and INT95 protospacer (illustrated by SEQ ID NO: 62) and the following two sequences for homologous integration: Sc_INT95B_FLANK5 (illustrated by SEQ ID NO: 63) and Sc_INT95B_FLANK3 (illustrated by SEQ ID NO: 64).

Diagnostic PCR was performed to confirm the correct assembly and integration at the INT95 locus of the promoted Pichia TKL1 expression cassette. Plasmid free colonies were selected and this resulted in new strains NX3 and NX4 which contains two copies of the promoted Pichia TKL1 expression cassette (see Table 8 for detailed genotypes).

### Example 4: Construction of New Strains NX5 and NX6

New strains NX5 and NX6 were constructed by transforming reference strain RX2 obtained in example 2 with four expression cassettes:

The first the cassette contains the DNA fragment named “fragmentA”, comprising the S. cerevisiae ANB1 promoter, Pichia pastoris TKL1 orf and the S. cerevisiae TDH1 terminator as referred to in example 3. (illustrated by SEQ ID NO: 50)

The second cassette contains a DNA fragment named “fragmentB”, comprising the S. cerevisiae MYO4 promoter (Sc_MYO4.pro), S. cerevisiae DAK1 orf (Sc_DAK1.orf) and S. cerevisiae GPM1 terminator (Sc_GPM1.ter). The nucleic acid sequence of the DNA fragment is illustrated in SEQ ID NO: 51.

The third cassette contains a DNA fragment named “fragmentC”, comprising the S. cerevisiae HHF2 promoter (Sc_HHF2.pro), E. coli gldA orf (Ec_gldA.orf) and S. cerevisiae EFM1 terminator (Sc_EFM1.ter). The nucleic acid sequence of the DNA fragment is illustrated in SEQ ID NO: 52.

The fourth cassette contains a DNA fragment named “fragmentD”, comprising the S. cerevisiae ANB1 promoter (Sc_ANB1.pro_0001), Zygosaccharomyces rouxiiorf encoding glycerol transporter GLYT (ZYROOE01210) (Zrou_T5.orf) and S. cerevisiae terminator (Sc_TEF1.ter_0001). The nucleic acid sequence of the DNA fragment is illustrated in SEQ ID NO: 53.

These four cassettes were integrated in the INT95 locus between SOD1 (YJR104C) and ADO1 (YJR105W) located on chromosome X of S. cerevisiae reference strain RX2 using CRISPR-Cas9 using the following sequences for homologous integration as described above with Sc_INT95B_FLANK5 (illustrated by SEQ ID NO: 63) and Sc_INT95B_FLANK3 (illustrated by SEQ ID NO: 64).

Diagnostic PCR was performed to confirm the correct assembly and integration at the INT95 locus of the four expression cassettes. Plasmid free colonies were selected which resulted in new strains NX5 and NX6 (see Table 8 for detailed genotypes).

### Example 5: Construction of New Strains NX7 and NX8

New strains NX7 and NX8 were constructed by transforming reference strain RX2 obtained in example 2 with the cassette containing the DNA fragment comprising S. cerevisiae ANB1 promoter, Pichia pastoris TKL1 orf and the S. cerevisiae TDH1 terminator, i.e. “fragmentA” (illustrated by SEQ ID NO: 50) as referred to in example 3, in the INT95 locus using CRISPR-Cas9 using Sc_INT95B_FLANK5.acc (SEQ ID NO: 63) and Sc_INT95B_FLANK3.acc (SEQ ID NO: 64) for homologous integration as also illustrated in example 3.

The GPD2 was deleted using CRISPR-Cas9 and GPD2 protospacer (illustrated by SEQ ID NO: 65) and for and a repair DNA fragment (illustrated by SEQ ID NO: 55) for homologous recombination and deleting GPD2 (illustrated by SEQ ID NO: 60). The INT95 integration and GPD2 deletion was checked by diagnostic PCR. Plasmid free colonies were selected which resulted in new strains NX7 and NX8 (see Table 8 for detailed genotypes).

### Example 6: Construction of New Strains NX9 and NX10

New strains NX9 and NX10 were constructed by transforming reference strain RX2 with three expression cassettes:

The first the cassette contains the DNA fragment named “fragmentA”, comprising the S. cerevisiae ANB1 promoter, Pichia pastoris TKL1 orf and the S. cerevisiae TDH1 terminator as referred to in example 3. (illustrated by SEQ ID NO: 50)

The second cassette contains a DNA fragment named “fragmentE” comprising S. cerevisiae PFY1promoter (Sc_PFY1.pro), E. coli gldA orf (Ec_gldA.orf) and S. cerevisiae EFM1 terminator (Sc_EFM1.ter). The nucleic acid sequence of the DNA fragment is illustrated in SEQ ID NO: 54

The third cassette contains the DNA fragment named “fragmentD”, comprising the S. cerevisiae ANB1 promoter (Sc_ANB1.pro_0001), Zygosaccharomyces rouxiiorf encoding glycerol transporter GLYT (ZYROOE01210) (Zrou_T5.orf) and S. cerevisiae terminator (Sc_TEF1.ter_0001). The nucleic acid sequence of the DNA fragment is illustrated in SEQ ID NO: 53.

These three cassettes were integrated in the INT95 locus using CRISPR-Cas9 using INT95B_FLANK5 (SEQ ID NO: 63) and INT95B_FLANK3 (SEQ ID NO: 64) for homologous integration. Diagnostic PCR was performed to confirm the correct assembly and integration at the INT95 locus of the three expression cassettes. Plasmid free colonies were selected which resulted in strain NX9 and NX10 (see Table 8 for detailed genotypes).

TABLE 8
S. cerevisiae strains used in this study

| Strain name | Genotype |
|---|---|
| Parental strain | Wildtype |
| intermediate strain IX1 | INT1::GroES-cbbM-GroEL |
| reference strain RX2 | INT1::GroES-cbbM-GroEL |
| | INT70::PRK-cbbM |
| NX3 | INT1::GroES-cbbM-GroEL |
| | INT70::PRK-cbbM |
| | INT95::pp_TKL1 |
| NX4 | INT1::GroES-cbbM-GroEL |
| | INT70::PRK-cbbM |
| | INT95::pp_TKL1 |
| NX5 | INT1::GroES-cbbM-GroEL |
| | INT70::PRK-cbbM |
| | INT95::ppTKL1-DAK1-gldA-T5 |
| NX6 | INT1::GroES-cbbM-GroEL |
| | INT70::PRK-cbbM |
| | INT95::ppTKL1-DAK1-gldA-T5 |
| NX7 | INT1::GroES-cbbM-GroEL |
| | INT70::PRK-cbbM |
| | INT95::ppTKL1-DAK1-gldA-T5 |
| | medium ΔGPD2 |
| NX8 | INT1::GroES-cbbM-GroEL |
| | INT70::PRK-cbbM |
| | INT95::ppTKL1-DAK1-gldA-T5 |
| | medium ΔGPD2 |
| NX9 | INT1::GroES-cbbM-GroEL |
| | INT70::PRK-cbbM |
| | INT95::ppTKL1-gldA-T5 |
| NX10 | INT1::GroES-cbbM-GroEL |
| | INT70::PRK-cbbM |
| | INT95::ppTKL1-gldA-T5 |

### Example 7: Fermentations

Precultures of the above new “NX” strains were made as follows: Glycerol stocks (−80° C.) were thawed at room temperature and used to inoculate 0.2 L mineral medium [as described by Luttik, M L H. et al (2000) “The Saccharomyces cerevisiae ICL2 Gene Encodes a Mitochondrial 2-Methyl isocitrate Lyase Involved in Propionyl-Coenzyme A Metabolism”. J. Bacteriol. 182:7007-13] supplemented with 2% (w/v) glucose, at pH 6.0 (adjusted with 2M H2SO4/4N KOH), in an unbaffled 0.5 L shake-flask. The precultures were incubated for 18 hours at 32° C. and shaken at 200 RPM. After estimating of the yeast cell dry weight (CDW) through OD600 measurement (using an existing CDW vs OD600 calibration line), a quantity of preculture corresponding to the required 0.5 g CDW/L inoculum concentration for the propagation was centrifuged (3 min, 5300×g), washed once with one sample volume sterile demineralized water, centrifuged once more, and resuspended in propagation medium.

Propagation of the above NX strains was carried out as follows: A propagation step was performed in 500 mL shake flasks using 100 mL of filtered and diluted corn mash (70% v/v Corn mash: 30% v/v water) supplemented with 1.25 g/L urea and the antibiotics: neomycin and penicillin G with a final concentration of 50 Ng/mL and 100 Ng/mL respectively. After all additions, the pH was adjusted to 5.0 using 2M H2SO4/4N KOH. Glucoamylase (Achieve®T, Novozymes, was dosed at the start of the propagation at a concentration of 0.1 mL/L. All strains were propagated for 6 h at 32° C. and shaken at 200 RPM.

Main fermentations of the above NX strains was carried out as follows: A main fermentation step was performed using 200 ml medium in 500 ml Schott bottles equipped with pressure recording/releasing caps (Ankom Technology, Macedon NY, USA), while shaking at 140 rpm and 32° C. pH was not controlled during fermentation. Fermentations were executed with corn mash having increased dry solids content of 36% w/w DS. Subsequently, the corn mash was supplemented with 1.0 g/L urea, and the antibiotics: neomycin and penicillin G with a final concentration of 50 μg/mL and 100 μg/mL respectively; antifoam (Basildon, approximately 0.5 mL/L). After all additions, the pH was adjusted to 5.0 using 2M H2SO4/4N KOH. Glucoamylase (Achieve®T, Novozymes) was dosed at the start of the fermentation at a concentration of 0.24 mL/L. The required yeast pitch from propagation to fermentation was 1.5% on fermentation volume. All strains were tested under a condition of high solids, i.e. 36% w/w DS).

Sampling of the fermentation was carried out as follows: Samples were taken from the main fermentations only. Samples for HPLC analysis were taken at 18, 24, 42, 48, and 66 hours. Table 9 summarizes the ethanol production (g/l) at each point in time. Table 10 summarizes the remaining glucose concentration (g/l) at each point in time.

Conclusions were as follows: The remaining glucose concentration was an indicator for the robustness of the yeast strain. Due to the presence of glucoamylase, glucose was continuously produced. Without wishing to be limited by any kind of theory it is believed that less robust strains will become more inhibited towards the end of the fermentation and as a result a higher concentration of unconverted glucose will be identified in the sample. A more robust strain will become less inhibited towards the end of the fermentation and as a result a lower concentration of unconverted glucose will be identified in the sample.

As illustrated by Table 10, the yeasts according to the invention, NX3, NX4, NX5, NX6, NX7, NX8, NX9 and NX10, resulted in lower concentrations of unconverted glucose after 48 hours and 66 hours of fermentation than the reference strain.

TABLE 9
Ethanol concentration (g/l) in the fermentation medium

| Time (h)/Strain | RX2 | NX3 | NX4 | NX5 | NX6 | NX7 | NX8 | NX9 | NX10 |
|---|---|---|---|---|---|---|---|---|---|
| 0 hours | 0.395 | 0.37 | 0.363 | 0.363 | 0.378 | 0.372 | 0.364 | 0.364 | 0.342 |
| 18 hours | 61.80 | 61.80 | 61.20 | 61.80 | 58.80 | 61.80 | 63.00 | 56.60 | 59.00 |
| 24 hours | 86.80 | 86.00 | 79.00 | 82.00 | 80.20 | 177.2 | 56.00 | 81.00 | 82.60 |
| 42 hours | 134.20 | 137.40 | 137.60 | 135.30 | 132.80 | 137.20 | 137.60 | 133.20 | 135.70 |
| 48 hours | 133.30 | 143.70 | 145.30 | 140.20 | 141.40 | 141.30 | 143.00 | 139.10 | 138.60 |
| 66 hours | 141.20 | 150.10 | 143.40 | 143.70 | 149.60 | 144.90 | 143.70 | 142.80 | 144.10 |
| Percentage (%) increase | 0.0 | 7.8 | 9.0 | 5.2 | 6.1 | 6.0 | 7.3 | 4.4 | 4.0 |
| in ethanol yield (g/l) | | | | | | | | | |
| vis-à-vis RX2 at 48 hours. | | | | | | | | | |
| Percentage (%) increase | 0.0 | 6.3 | 1.6 | 1.8 | 5.9 | 2.6 | 1.8 | 1.1 | 2.1 |
| in ethanol yield (g/l) | | | | | | | | | |
| vis-à-vis RX2 at 66 hours. | | | | | | | | | |

TABLE 10
Remaining glucose (grams/liter) concentrations in the fermentation medium

| Time/Strain | RX2 | NX3 | NX4 | NX5 | NX6 | NX7 | NX8 | NX9 | NX10 |
|---|---|---|---|---|---|---|---|---|---|
| 0 hours | 40.82 | 40.80 | 40.81 | 40.88 | 40.86 | 40.90 | 40.92 | 40.89 | 40.94 |
| 18 hours | 59.80 | 51.80 | 50.80 | 61.80 | 60.60 | 46.20 | 45.60 | 64.60 | 58.00 |
| 24 hours | 50.00 | 37.80 | 37.80 | 52.60 | 48.60 | 70.2 | 24.50 | 52.00 | 44.00 |
| 42 hours | 15.00 | 6.44 | 5.22 | 13.50 | 17.90 | 5.72 | 5.18 | 15.50 | 9.79 |
| 48 hours | 13.80 | 6.33 | 4.55 | 9.42 | 10.71 | 5.42 | 4.56 | 10.13 | 7.10 |
| 66 hours | 18.80 | 13.16 | 10.01 | 14.30 | 14.50 | 11.76 | 11.25 | 14.60 | 12.73 |
| Percentage (%) decrease | 0.0 | −54.1 | −67.0 | −31.7 | −22.4 | −60.7 | −67.0 | −26.6 | −48.6 |
| in remaining glucose (g/l) | | | | | | | | | |
| vis-à-vis RX2 at 48 hours. | | | | | | | | | |
| Percentage (%) decrease | 0.0 | −30.0 | −46.8 | −23.9 | −22.9 | −37.4 | −40.2 | −22.3 | −32.3 |
| in remaining glucose (g/l) | | | | | | | | | |
| vis-à-vis RX2 at 66 hours. | | | | | | | | | |

### REFERENCES

Entian K D, Kötter P. Yeast genetic strain and plasmid collections. Method Microbiol. 2007; 629-66. Nijkamp J F, van den Broek M, Datema E, de Kok S, Bosman L, Luttik M A, Daran-Lapujade P, Vongsangnak W, Nielsen J, Heijne W H M, Klaassen P, Paddon C J, Platt D, Kötter P, van Ham R C, Reinders M J T, Pronk J T, de Ridder D, Daran J-M. De novo sequencing, assembly and analysis of the genome of the laboratory strain Saccharomyces cerevisiae CEN.PK113-7D, a model for modern industrial biotechnology. Microb Cell Fact. 2012; 11:36.
Verduyn C, Postma E, Scheffers W A, van Dijken J P. Effect of benzoic acid on metabolic fluxes in yeasts: A continuous-culture study on the regulation of respiration and alcoholic fermentation. Yeast. 1992; 8:501-17.
Mans R, van Rossum H M, Wijsman M, Backx A, Kuijpers N G, van den Broek M, Daran-Lapujade P, Pronk J T, van Maris A J A, Daran J-M. CRISPR/Cas9: a molecular Swiss army knife for simultaneous introduction of multiple genetic modifications in Saccharomyces cerevisiae. FEMS Yeast Res. 2015; 15:fov004.
DiCarlo J E, Norville J E, Mali P, Rios X, Aach J, Church G M. Genome engineering in Saccharomyces cerevisiae using CRISPR-Cas systems. Nucleic Acids Res. 2013; 1-8.
Mikkelsen M D, Buron L D, Salomonsen B, Olsen C E, Hansen B G, Mortensen U H, Halkier B A. Microbial production of indolylglucosinolate through engineering of a multi-gene pathway in a versatile yeast expression platform. Metab Eng. 2012; 14:104-11.
Knijnenburg T A, Daran J M, van den Broek M A, Daran-Lapujade P A, de Winde J H, Pronk J T, Reinders M J, Wessels L F. Combinatorial effects of environmental parameters on transcriptional regulation in Saccharomyces cerevisiae: A quantitative analysis of a compendium of chemostat-based transcriptome data. BMC Genomics. 2009; 10:53.
Mumberg D, Müller R, Funk M. Yeast vectors for the controlled expression of heterologous proteins in different genetic backgrounds. Gene. 1995; 156:119-22.
Gueldener U, Heinisch J, Koehler G J, Voss D, Hegemann J H. A second set of loxP marker cassettes for Cre-mediated multiple gene knockouts in budding yeast. Nucleic Acids Res. 2002; 30:e23.
Guadalupe-Medina V, Wisselink H, Luttik M, de Hulster E, Daran J-M, Pronk J T, van Maris A J A. Carbon dioxide fixation by Calvin-Cycle enzymes improves ethanol yield in yeast. Biotechnol Biofuels. 2013; 6:125.
Daniel Gietz R, Woods R A: Transformation of yeast by lithium acetate/single-stranded carrier DNA/polyethylene glycol method. Methods Enzymol. 2002:87-96.
Solis-Escalante D, Kuijpers N G A, Bongaerts N, Bolat I, Bosman L, Pronk J T, Daran J-M, Daran-Lapujade P. amdSYM, a new dominant recyclable marker cassette for Saccharomyces cerevisiae. FEMS Yeast Res. 2013; 13:126-39.
Guadalupe-Medina V, Almering M J H, van Maris A J A, Pronk J T. Elimination of glycerol production in anaerobic cultures of a Saccharomyces cerevisiae strain engineered to use acetic acid as an electron acceptor. Appl Environ Microb. 2010; 76:190-5.
Papapetridis I, van Dijk M, Dobbe A P, Metz B, Pronk J T, van Maris A J A. Improving ethanol yield in acetate-reducing Saccharomyces cerevisiae by cofactor engineering of 6-phosphogluconate dehydrogenase and deletion of ALD6. Microb Cell Fact. 2016; 15:1-16.
Heijnen J J, van Dijken J P. In search of a thermodynamic description of biomass yields for the chemotrophic growth of microorganisms. Biotechnol Bioeng. 1992; 39:833-58.
Postma E, Verduyn C, Scheffers W A, van Dijken J P. Enzymic analysis of the crabtree effect in glucose-limited chemostat cultures of Saccharomyces cerevisiae. Appl Environ Microbiol. 1989; 55:468-77.
Verduyn C, Postma E, Scheffers W A, van Dijken J P. Physiology of Saccharomyces cerevisiae in anaerobic glucose-limited chemostat cultures. J Gen Microbiol. 1990; 136:395-403.
Kwast et al. Genomic Analysis of Anaerobically induced genes in Saccharomyces cerevisiae: Functional roles of ROX1 and other factors in mediating the anoxic response, 2002, Journal of bacteriology vol 184, no 1 p 250-265.
Keng, T. 1992. HAP1 and ROX1 form a regulatory pathway in the repression of HEM13 transcription in Saccharomyces cerevisiae. Mol. Cell. Biol. 12: 2616-2623.
Labbe-Bois, R., and P. Labbe. 1990. Tetrapyrrole and heme biosynthesis in the yeast Saccharomyces cerevisiae, p. 235-285. In H. A. Dailey (ed.), Biosynthesis of heme and chlorophylls. McGraw-Hill, New York, N.Y.
Zitomer, R. S., and C. V. Lowry. 1992. Regulation of gene expression by oxygen in Saccharomyces cerevisiae. Microbiol. Rev. 56:1-11.
Zitomer, R. S., P. Carrico, and J. Deckert. 1997. Regulation of hypoxic gene expression in yeast. Kidney Int. 51:507-513.
Cohen et al., Induction and repression of DAN1 and the family of anaerobic mannoprotein genes in Saccharomyces cerevisiae occurs through a complex array of regulatory sites. Nucleic Acid Research, 2001 Vol. 29, No 3, 799-808
Ter Kinde and de Steensma, A microarray-assisted screen for potential Hap1 and Rox1 target genes in Saccharomyces cerevisiae, 2002, Yeast 19: 825-840.
Sertil et al. The DAN1 gene of S. cerevisiae is regulated in parallel with the hypoxic gene, but by a different mechanism, 1997, Gene Vol 192, pag 199-205.
Nissen et al., “Anaerobic and aerobic batch cultivations of Saccharomyces cerevisiae mutants impaired in glycerol Synthesis”, (2000), Yeast, vol. 16, pages 463-474.
Sambrook et al., Molecular Cloning-A Laboratory Manual, 2nd ed., Vol. 1-3 (1989), published by Cold Spring Harbor Publishing.
Kruskal et al, “An overview of sequence comparison: Time warps, string edits, and macromolecules”, (1983), Society for Industrial and Applied Mathematics (SIAM), Vol 25, No. 2, pages 201-237.
D. Sankoff and J. B. Kruskal, (ed.), Time warps, string edits and macromolecules: the theory and practice of sequence comparison, pp. 1-44 Addison-Wesley Publishing Company.
Needleman et al “A General Method Applicable to the Search for Similarities in the Amino Acid Sequence of Two Proteins” (1970) J. Mol. Biol. Vol. 48, pages 443-453.
Sherman, F., et al., Methods in Yeast Genetics, Cold Spring Harbor Laboratory (1986)
Rice et al, “EMBOSS: The European Molecular Biology Open Software Suite” (2000), Trends in Genetics vol. 16, (6) pages 276-277, http://emboss.bioinformatics.nl/
Neves et al., “Yeast orthologues associated with glycerol transport and metabolism”, (2004), FEMS Yeast Res. Vol. 5, pages 51-62.
Neves et al “New insights on glycerol transport in Saccharomyces cerevisiae”, (2004), FEBS Letters 565 (2004) 160-162.
Kwast et al., “Genomic Analysis of Anaerobically induced genes in Saccharomyces cerevisiae: Functional roles of ROX1 and other factors in mediating the anoxic response”, (2002), Journal of bacteriology vol 184, no 1 pages 250-265.
Molin et al (2003) “Dihydroxy-acetone kinases in Saccharomyces cerevisiae are involved in detoxification of dihydroxyacetone” (2003), J. Biol. Chem., vol. 278: pages 1415-1423.
Guadalupe-Medina et al., “Carbon dioxide fixation by Calvin-Cycle enzymes improves ethanol yield in yeast”, published in Biotechnol, Biofuels, 2013, vol. 6, p. 125 onwards.
Yébenes et al., “Chaperonins: two rings for folding” (2011), Trends in Biochemical Sciences, Vol. 36, No. 8, pages 424-432.
Zeilstra-Ryalls et al., “The universally conserved GroE (Hsp60) chaperonins”, published in Annu Rev Microbiol. (1991) vol. 45, pages 301-25.
Horwich et al., “Two Families of Chaperonin: Physiology and Mechanism”, (2007), Annu. Rev. Cell. Dev. Biol. Vol. 23, pages 115-45.
Sonderegger et al., “Metabolic Engineering of a Phosphoketolase Pathway for Pentose Catabolism in Saccharomyces cerevisiae”, (2004), Applied & Environmental Microbiology, vol. 70(5), pages 2892-2897.
Membrillo-Hernandez et al., “Evolution of the adhE Gene Product of Escherichia coli from a Functional Reductase to a Dehydrogenase”, (2000) J. Biol. Chem. 275: pages 33869-33875.
Tamarit et al. “Identification of the Major Oxidatively Damaged Proteins in Escherichia coli Cells Exposed to Oxidative Stress” (1998) J. Biol. Chem. 273: pages 3027-3032.
Smith et al. “Purification, Properties, and Kinetic Mechanism of Coenzyme A-Linked Aldehyde Dehydrogenase from Clostridium kluyveri” (1980) Arch. Biochem. Biophys. 203: pages 663-675.
Toth et al. “The ald Gene, Encoding a Coenzyme A-Acylating Aldehyde Dehydrogenase, Distinguishes Clostridium beijerinckii and Two Other Solvent-Producing Clostridia from Clostridium acetobutylicum”, (1999), Appl. Environ. Microbiol. 65: pages 4973-4980.
Powlowski and Shingler “Genetics and biochemistry of phenol degradation by Pseudomonas sp. CF600”, (1994), Biodegradation vol. 5, pages 219-236.
Shingler et al., “Nucleotide Sequence and Functional Analysis of the Complete Phenol/3,4-Dimethylphenol Catabolic Pathway of Pseudomonas sp. Strain CF600”, (1992), J. Bacteriol., Vol. 174, pages 711-724.
Ferrandez et al., “Genetic Characterization and Expression in Heterologous Hosts of the 3-(3-Hydroxyphenyl) Propionate Catabolic Pathway of Escherichia coli K-12′ (1997) J. Bacteriol. 179: pages 2573-2581.
Lutstorf and Megnet, “Multiple Forms of Alcohol Dehydrogenase in Saccharomyces Cerevisiae”, (1968), Arch. Biochem. Biophys., vol. 126, pages 933-944.
Ciriacy, “Genetics of Alcohol Dehydrogenase in Saccharomyces cerevisiae I. Isolation and genetic analysis of adh mutants”, (1975), Mutat. Res. 29, pages 315-326.
Engler et al., “Generation of Families of Construct Variants Using Golden Gate Shuffling”, (2011), published in chapter 11 of Chaofu Lu et al. (eds.), cDNA Libraries: Methods and Applications, Methods in Molecular Biology, vol. 729, pages 167-180.
DiCarlo et al., “Genome engineering in Saccharomyces cerevisiae using CRISPR-Cas systems”, (2013), Nucleic Acids Res Vol 41, pages 4336-4343.
Sikorski and Hieter, “A System of Shuttle Vectors and Yeast Host Strains Designed for Efficient Manipulation of DNA in Saccharomyces cerevisiae”, (1989), Genetics, vol. 122, pages 19-27

1. A recombinant yeast cell functionally expressing:
a) a heterologous nucleic acid sequence encoding a protein having ribulose-1,5-phosphate carboxylase/oxygenase activity (EC4.1.1.39; Rubisco), a heterologous nucleic acid sequence encoding a protein having phosphoribulokinase activity (EC2.7.1.19; PRK) and one or more nucleic acid sequences encoding for molecular chaperones for the protein having ribulose-1,5-phosphate carboxylase/oxygenase activity; and
b) a nucleic acid sequence encoding a protein having transketolase activity (EC 2.2.1.1),
wherein the expression of the nucleic acid sequence encoding the protein having transketolase activity is under control of a promoter (the “TKL promoter”), which TKL promoter has an anaerobic/aerobic expression ratio for the transketolase of 2 or more.

2. The recombinant yeast cell according to claim 1, wherein the TKL promoter is the promoter of a gene selected from the list consisting of: FET4, ANB1, YHR048W, DAN1, AAC3, TIR2, DIP5, HEM13, YNR014W, YAR028W, FUN 57, COX5B, OYE2, SUR2, FRDS1, PIS1, LAC1, YGR035C, YAL028W, EUG1, HEM14, ISU2, ERG26, YMR252C, SML1, TIR2, TIR4, TIR3, PAU7, PAU5, YLL064C, YGR294W, DAN3, YIL176C, YGL261C, YOL161C, PAU1, PAU6, DAN2, YDR542W, YIR041W, YKL224C, PAU3, YLL025W, YOR394W, YHL046C, YMR325W, YAL068C, YPL282C, PAU2, and PAU4.

3. The recombinant yeast strain according to claim 1, wherein the TKL promoter is a synthetic oligonucleotide.

4. The recombinant yeast cell according to claim 1, wherein a native nucleic acid sequence encoding for a protein having transketolase activity is under control of the TKL promoter.

5. The recombinant yeast cell according to claim 1, wherein the recombinant yeast cell functionally expresses a heterologous nucleic acid sequence encoding a protein having transketolase activity.

6. The recombinant yeast cell according to claim 5, wherein the protein having transketolase activity comprises or consists of:
an amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27; or
a functional homologue of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27 at least 70% sequence identity with the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27; or
a functional homologue of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27 having one or more mutations, substitutions, insertions and/or deletions when compared with the amino acid sequence of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 or SEQ ID NO: 27.

7. The recombinant yeast cell according to claim 5, wherein the heterologous nucleic acid sequence encoding for the protein having transketolase activity is under control of the TKL promoter.

8. The recombinant yeast cell according to claim 5, wherein the recombinant yeast cell is a recombinant Saccharomyces cerevisiae yeast cell, functionally expressing a heterologous nucleic acid sequence encoding a protein having transketolase activity, wherein:
the protein having transketolase activity comprises or consists of an amino acid sequence having in the range of equal to or more than 30% to equal to or less than 80% sequence identity with the amino acid sequence of SEQ ID NO: 9; or
the heterologous nucleic acid sequence comprises or consists of a nucleic acid sequence having in the range of equal to or more than 30% to equal to or less than 80% sequence identity with the nucleic acid sequence of SEQ ID NO: 10.

9. The recombinant yeast cell according to claim 5, wherein a native nucleic acid sequence encoding for a protein having transketolase activity has been disrupted or deleted.

10. The recombinant yeast cell according claim 5, wherein the recombinant yeast cell comprises the heterologous nucleic acid sequence encoding for the protein having transketolase activity in addition to a native nucleic acid sequence encoding for a protein having transketolase activity.

11. The recombinant yeast cell according to claim 1, wherein the nucleic acid sequence encoding for a ribulose-1,5-phosphate carboxylase/oxygenase is under a constitutive promoter.

12. The recombinant yeast cell according to claim 1, wherein the recombinant yeast cell comprises in the range from equal to or more than 1 to equal to or less than 7 copies of the nucleic acid sequence encoding for the ribulose-1,5-phosphate carboxylase/oxygenase.

13. The recombinant yeast cell according to claim 1, wherein the recombinant yeast cell further functionally expresses:
a nucleic acid sequences encoding for a glycerol dehydrogenase (E.C. 1.1.1.6);
a nucleic acid sequences encoding for a dihydroxyacetone kinase (E.C. 2.7.1.28 or E.C. 2.7.1.29); and
a nucleic acid sequences encoding for a glycerol transporter.

14. The recombinant yeast cell according to claim 1, wherein the recombinant yeast cell further functionally expresses a nucleic acid sequence encoding for a glucoamylase (EC 3.2.1.20 or 3.2.1.3).

15. A process for the production of ethanol, comprising converting a carbon source using a recombinant yeast cell according to claim 1.

16. The process according to claim 15, wherein the process is at least partly carried out in a medium comprising glucose in a glucose concentration of 25 g/L or more.

17. The process according to claim 15, wherein the process is at least partly carried out in the presence of a saccharolytic enzyme.

18. The process according to claim 17, wherein the saccharolytic enzyme is a glucoamylase.


SEQUENCE LISTING (65 sequences)

SEQ ID NO: 1 - protein, 459 aa, Thiobacillus denitrificans
  REGION 1..459: note = Ribulose-1,5-biphosphate carboxylase oxygenase (RuBisCO)
MDQSARYADLSLKEEDLIKGGRHILVAYKMKPKSGYGYLEAAAHFAAESSTGTNVEVSTTDDFTKGVDALVYYIDEASEDMRIAYPLELFDRNVTDGRFMLVSFLTLAIGNNQGMGDIEHAKMIDFYVPERCIQMFDGPATDISNLWRILGRPVVNGGYIAGTIIKPKLGLRPEPFAKAAYQFWLGGDFIKNDEPQGNQVFCPLKKVLPLVYDAMKRAQDDTGQAKLFSMNITADDHYEMCARADYALEVFGPDADKLAFLVDGYVGGPGMVTTARRQYPGQYLHYHRAGHGAVTSPSAKRGYTAFVLAKMSRLQGASGIHVGTMGYGKMEGEGDDKIIAYMIERDECQGPVYFQKWYGMKPTTPIISGGMNALRLPGFFENLGHGNVINTAGGGSYGHIDSPAAGAISLRQSYECWKQGADPIEFAKEHKEFARAFESFPKDADKLFPGWREKLGVHS

SEQ ID NO: 2 - DNA, 1380 bp, synthetic construct
  misc_feature 1..1380: note = synthetic; based on cbbM gene from Thiobacillus denitrificans -pBTWW002, codon optimized for S. cerevisiae
  source 1..1380: mol_type = other DNA
atggatcaatctgcaagatatgctgacttgtctttaaaggaagaagatttgattaaaggtggtagacatattttggttgcttacaaaatgaaaccaaaatctggttatggttatttggaagctgctgctcattttgctgctgaatcttctacaggtacaaatgttgaagtttctactacagatgattttacaaaaggtgttgatgctttagtttactacatcgatgaagcttcagaagatatgagaattgcttatccattggaattattcgacagaaatgttactgacggaagattcatgttagtttcttttttgactttggctattggtaacaatcaaggaatgggagatatagaacatgcaaaaatgatagatttttacgttccagaaagatgtattcaaatgtttgatggtccagctacagatatttctaatttgtggagaattttgggtagaccagtagttaatggtggttatattgctggtactattattaagccaaaattgggtttaagaccagaaccatttgctaaagctgcttatcaattttggttgggtggagattttatcaagaatgacgaaccacaaggtaatcaagttttttgtccattgaaaaaagttttgccattggtttacgatgctatgaaaagagcacaagatgatactggtcaagcaaaattgttttctatgaatattactgcagacgatcattatgaaatgtgtgcaagagctgattatgctttggaagttttcggtccagatgcagataaattggcttttttggtagatggttacgttggaggtccaggaatggttactactgctagaaggcaatatcctggtcaatatttgcattatcatagagcaggtcacggtgctgttacttctccatctgctaaaagaggttatactgcttttgttttggctaaaatgtctagattgcaaggcgcttcaggtattcatgttggtactatgggttatggaaaaatggaaggagaaggcgacgataagattattgcttatatgatagaaagggacgaatgtcaaggtccagtttattttcaaaaatggtacggtatgaaaccaactactccaattatctccggaggaatgaatgctttgagattgcctggttttttcgaaaatttgggtcatggtaacgttattaatactgcaggtggtggttcttacggtcatattgattctcctgctgctggtgctatttctttgagacaatcttacgaatgttggaaacaaggtgcagatccaattgaatttgctaaggaacataaggaatttgcaagagcttttgaatcttttccaaaagatgctgataagttatttccaggatggagagaaaaattgggagttcattcttaa

SEQ ID NO: 3 - protein, 352 aa, Spinacia oleracea
  REGION 1..352: note = S. oleracea phosphoribulokinase (prk)
MSQQQTIVIGLAADSGCGKSTFMRRLTSVFGGAAEPPKGGNPDSNTLISDTTTVICLDDFHSLDRNGRKVEKVTALDPKANDFDLMYEQVKALKEGKAVDKPIYNHVSGLLDPPELIQPPKILVIEGLHPMYDARVRELLDFSIYLDISNEVKFAWKIQRDMKERGHSLESIKASIESRKPDFDAYIDPQKQHADVVIEVLPTELIPDDDEGKVLRVRMIQKEGVKFFNPVYLFDEGSTISWIPCGRKLTCSYPGIKFSYGPDTFYGNEVTVVEMDGMFDRLDELIYVESHLSNLSTKFYGEVTQQMLKHQNFPGSNNGTGFFQTIIGLKIRDLFEQLVASRSTATATAAKA

SEQ ID NO: 4 - DNA, 1059 bp, synthetic construct
  misc_feature 1..1059: note = synthetic; based on prk gene from S. oleracea - codon optimizedfor S. cerevisiae
  source 1..1059: mol_type = other DNA
atgtcacaacaacaaacaattgtgattggtttagcagcagattcaggttgtggtaagagtacattcatgaggaggttaacaagtgttttcggtggcgcggccgagccaccaaagggtggtaacccagattcaaacacattgattagtgacactactactgttatctgtttggatgattttcattcccttgatagaaatggcaggaaagtggaaaaagttactgctttagacccaaaagctaatgattttgatcttatgtatgaacaagttaaggctttgaaagaaggtaaagctgttgataaacctatttataatcatgtttctggtttgttggaccctcctgagcttattcaacctcctaagatcttggtcattgaagggttacaccccatgtatgacgcacgtgtgagggaattgctagacttcagcatctacttggacattagcaatgaagttaaatttgcctggaaaattcagagagacatgaaagaaagaggacacagtcttgaaagcatcaaagccagtattgaatccagaaagccagattttgatgcttacattgacccacaaaagcagcatgctgatgtagtgattgaagtattgccaactgaactcattcctgatgatgatgaaggcaaagtgttgagagtaaggatgattcagaaagaaggagtcaagtttttcaacccagtttacttgtttgatgaaggatctaccatttcatggattccatgtggtagaaaattaacatgttcttaccctggtatcaaattttcctatggcccagacaccttctatggcaacgaggtgacagtagtagagatggatgggatgtttgacagattagacgaactaatctacgtcgaaagccatttgagcaatctatcaaccaagttttatggtgaagtcactcaacaaatgttgaagcaccaaaatttcccaggaagcaacaatggaactggtttcttccaaaccataattggattgaagatcagagacttgttcgagcagctcgttgctagcaggtctacagcaactgcaacagctgctaaagcctag

SEQ ID NO: 5 - protein, 548 aa, synthetic construct
  REGION 1..548: note = Translated protein sequence, based on GroEL from E. coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 6 - DNA, 1647 bp, synthetic construct
  misc_feature 1..1647: note = Synthetic, based on GroEL from E. coli - codon optimized
  source 1..1647: mol_type = other DNA
atggctgctaaggacgttaagttcggtaacgacgctagagttaagatgttgagaggtgttaacgttttggctgacgctgttaaggttactttgggtccaaagggtagaaacgttgttttggacaagtctttcggtgctccaactatcactaaggacggtgtttctgttgctagagaaatcgaattggaagacaagttcgaaaacatgggtgctcaaatggttaaggaagttgcttctaaggctaacgacgctgctggtgacggtactactactgctactgttttggctcaagctatcatcactgaaggtttgaaggctgttgctgctggtatgaacccaatggacttgaagagaggtatcgacaaggctgttactgctgctgttgaagaattgaaggctttgtctgttccatgttctgactctaaggctatcgctcaagttggtactatctctgctaactctgacgaaactgttggtaagttgatcgctgaagctatggacaaggttggtaaggaaggtgttatcactgttgaagacggtactggtttgcaagacgaattggacgttgttgaaggtatgcaattcgacagaggttacttgtctccatacttcatcaacaagccagaaactggtgctgttgaattggaatctccattcatcttgttggctgacaagaagatctctaacatcagagaaatgttgccagttttggaagctgttgctaaggctggtaagccattgttgatcatcgctgaagacgttgaaggtgaagctttggctactttggttgttaacactatgagaggtatcgttaaggttgctgctgttaaggctccaggtttcggtgacagaagaaaggctatgttgcaagacatcgctactttgactggtggtactgttatctctgaagaaatcggtatggaattggaaaaggctactttggaagacttgggtcaagctaagagagttgttatcaacaaggacactactactatcatcgacggtgttggtgaagaagctgctatccaaggtagagttgctcaaatcagacaacaaatcgaagaagctacttctgactacgacagagaaaagttgcaagaaagagttgctaagttggctggtggtgttgctgttatcaaggttggtgctgctactgaagttgaaatgaaggaaaagaaggctagagttgaagacgctttgcacgctactagagctgctgttgaagaaggtgttgttgctggtggtggtgttgctttgatcagagttgcttctaagttggctgacttgagaggtcaaaacgaagaccaaaacgttggtatcaaggttgctttgagagctatggaagctccattgagacaaatcgttttgaactgtggtgaagaaccatctgttgttgctaacactgttaagggtggtgacggtaactacggttacaacgctgctactgaagaatacggtaacatgatcgacatgggtatcttggacccaactaaggttactagatctgctttgcaatacgctgcttctgttgctggtttgatgatcactactgaatgtatggttactgacttgccaaagaacgacgctgctgacttgggtgctgctggtggtatgggtggtatgggtggtatgggtggtatgatgtaa

SEQ ID NO: 7 - protein, 97 aa, synthetic construct [sequence omitted]

SEQ ID NO: 8 - DNA, 294 bp, synthetic construct
  misc_feature 1..294: note = Synthetic, based on GroES from E. coli - codon optimized
  source 1..294: mol_type = other DNA
atgaacatcagaccattgcacgacagagttatcgttaagagaaaggaagttgaaactaagtctgctggtggtatcgttttgactggttctgctgctgctaagtctactagaggtgaagttttggctgttggtaacggtagaatcttggaaaacggtgaagttaagccattggacgttaaggttggtgacatcgttatcttcaacgacggttacggtgttaagtctgaaaagatcgacaacgaagaagttttgatcatgtctgaatctgacatcttggctatcgttgaagcttaa

SEQ ID NO: 9 - protein, 680 aa, Saccharomyces cerevisiae
  REGION 1..680: note = Transketolase 1 (TKL1) from Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 10 - DNA, 2059 bp, Saccharomyces cerevisiae
  misc_feature 1..2059: note = Transketolase 1 (TKL1) from Saccharomyces cerevisiae
  source 1..2059: mol_type = unassigned DNA
ggtctcgaatgactcaattcaccgatatcgacaaattggctgtctccaccattagaattttggctgttgacactgtttccaaggctaactctggtcatccaggtgccccattaggtatggctccagctgctcacgtcttatggtcccaaatgagaatgaacccaaccaacccagattggatcaacagagacagattcgttttgtctaacggtcacgctgttgctctattatactccatgttgcatttgactggttacgatttgtctatcgaagacttgaagcaattcagacaattgggttccagaactccaggtcaccctgaattcgaactaccaggtgtcgaagtcaccactggtccattaggtcaaggtatctccaatgctgtcggtatggctatggcccaagctaacttagctgccacttacaacaagccaggtttcaccttgtctgacaactacacttacgttttcttgggtgatggttgtttgcaagaaggtatttcttctgaagcttcttctttggctggtcacttgaaattgggtaacttgattgccatctacgatgacaacaagatcactatcgatggtgctacttccatttctttcgacgaagacgttgccaagagatacgaagcttacggttgggaagtcttgtacgttgaaaacggtaacgaagacttagccggtattgccaaggccattgctcaagctaagttgtccaaggacaagccaactttgatcaagatgaccaccaccattggttacggttctttgcacgctggttctcactccgttcacggtgctccattgaaggctgacgatgtcaagcaattgaagtccaagttcggtttcaacccagacaagtctttcgttgtcccacaagaagtctacgaccattatcaaaagaccatcttgaaaccaggtgttgaagctaacaacaaatggaacaaattgttttccgaataccaaaagaagttcccagaattgggtgctgaattggccagaagattgtctggtcaattgccagctaactgggaatccaagttgccaacctacactgccaaggactctgctgttgctaccagaaagttgtctgaaaccgttttggaagatgtctacaaccaattaccagaattgattggtggttctgctgatttgaccccatccaacttgaccagatggaaggaagctttggatttccaaccaccatcttccggttctggtaactactccggtagatacatcagatacggtatcagagaacacgctatgggtgccattatgaacggtatttctgccttcggtgctaactacaagccttacggtggtactttcttgaactttgtttcctacgctgctggtgctgttcgtttgtctgctttgtccggtcacccagtcatctgggtcgccactcacgactctatcggtgtcggtgaagacggtccaacccaccaaccaattgaaactttagctcacttcagatccttgccaaacatccaagtctggcgtccagctgacggtaatgaagtctctgctgcctacaagaactctttggaatctaagcacactccatctatcatcgctttgtccagacaaaacttgcctcaattggaaggttcttctatcgaatctgcttccaagggtggttacgtcttacaagatgttgctaacccagatatcattttggttgctactggttctgaagtttctctatccgtcgaagctgctaagactttggctgccaagaacatcaaggccagagttgtctctttgccagatttcttcaccttcgacaagcaaccattggaatacagattatctgttttgccagacaatgtcccaatcatgtccgttgaagttttggctaccacctgttggggtaagtacgctcaccaatctttcggtattgaccgtttcggtgcttctggtaaggctccagaagttttcaaattcttcggttttaccccagaaggtgttgctgaaagagctcaaaagactatcgctttctacaagggtgacaaattgatctccccattgaagaaagcgttttaaaggagacc

SEQ ID NO: 11 - protein, 707 aa, Komagataella phaffii
  REGION 1..707: note = Transketolase 1 (TKL1) from Komagataella phaffii 7435
MARIPKAVSYNDDIHDLVIKTFRCYVLDLVEQYGGGHPGSAMGMVAIGIALWKYQMKYAPNDPDYFNRDRFVLSNGHVCLFQYLFQHLTGLKEMTVKQLQSYHSSDYHSLTPGHPEIENPAVEVTTGPLGQGISNAVGMAIGSKNLAATYNRPGFPVVDNTIYAIVGDACLQEGPALESISLAGHLALDNLIVIYDNNQVCCDGSVDVNNTEDISAKFRAQNWNVIDIVDGSRDVATIVKAIDWAKAETERPTLINVRTEIGQDSAFGNHHAAHGSALGEEGIRELKTKYGFNPAQKFWFPKEVYDFFAEKPAKGDELVKNWKKLVDSYVKEYPREGQEFLSRVRGELPKNWRTYIPQDKPTEPTATRTSAREIVRALGKNLPQVIAGSGDLSVSILLNWDGVKYFFNPKLQTFCGLGGDYSGRYIEFGIREHSMCAIANGLAAYNKGTFLPITSTFYMFYLYAAPALRMAALQELKAIHIATHDSIGAGEDGPTHQPIALSSLFRAMPNFYYMRPADATEVAALFEVAVELEHSTLLSLSRHEVDQYPGKTSAQGAKRGGYVVEDCEGKPDVQLIGTGSELEFAIKTARLLRQQKGWKVRVLSFPCQRLFDEQSITYRRSVLRRGEVPTVVVEAYVAYGWERYATAGYTMNTFGKSLPVEDVYKYFGYTPEKIGERVVQYVNSIKASPQILYEFHDLKGKPKHDKL

SEQ ID NO: 12 - protein, 707 aa, Komagataella phaffii
  REGION 1..707: note = Transketolase 2 (TKL2) from Komagataella phaffii 7435
MARIPKAVSTQDDIHELVIKTFRCYVLDLVEQYGGGHPGSAMGMVAIGIALWKYQMKYAPNDPDYFNRDRFVLSNGHVCLFQYLFQHLTGLKEMTVKQLQSYHSSDYHSLTPGHPEIENPAVEVTTGPLGQGISNAVGMAIGSKNLAATYNRPGFPVVDNTIYAIVGDACLQEGPALESISLAGHLALDNLIVIYDNNQVCCDGSVDVNNTEDISAKFRAQNWNVIEVENGSRDVATLVKAIEWAKAENERPTLINVRTEIGQDSAFGNHHAAHGSALGEEGIRELKAKYGFDVARKFWFPQEVYDFFAEKPAEGDQLVANWKKLLDEYVKNYPQEGEELKARIRGELPKNWKSFIPQDKPTEPTATRTSAREIVRSLGQNLPQVIAGSGDLSVSILLNWGGVKYFFNPKLQTFCGLGGDYSGRYIEFGIREHSMCAIANGLAAYNKGTFLPITSTFYMFYLYAAPALRMAALQELKAIHIATHDSIGAGEDGPTHQPIALSSLFRAMPNFYYIRPADATEVAALFEVAVELEHSTLFSLSRHEVEQYPGKTSAEGAKRGGYVVEDCEGKPDVQLIGAGSELEFAVKTARLLRQQKGWKVRVLSFPCQRLFDQQSLAYRRSVLRRGEVPTVVVEAYVAYGWERYATAGYTMNTFGKSLPVEDVYKYFGYTPEKIGEKVAAYVNSIKASPQILYEFTDLKGKPKHDKL

SEQ ID NO: 13 - protein, 676 aa, Candida tenuis
  REGION 1..676: note = Transketolase from Candida tenuis (strain ATCC 10573)
MSTDQLAVNTIRLLAVDTVSKANSGHPGAPLGLAPAAHVVFQNMKFNPKDTKWINRDRFVLSNGHACALLYTMLFLYGYDYTVEDLKSFRQLHSKTPGHPESTDCPGVEVTTGPLGQGICNAVGLALAQKQFAATYNKPNYTLSDNFTYCFLGDGCLMEGVSSEASSLAGHLQLGNLIAFWDDNQISIDGSTEVAFTEDVIARYKAYAWHILEVEDGNSDLAGIAKAIEEAKKVTDKPTLIRIKTTIGYGSLAQGTHGVHGAPLKPDDVKQLKSKFGFDPEEFFAVPKEVTESYQKHVESNLKVQKEWEATFAAYKKEYPELGAEVQRRLDGKLPEGWEKALPSFTPADKSVATRKLSEGVINALSDILPEFIGGSADLTPSNLTKATGSVDFQPPSTGLGDYSGKYIRYGVREHGMGAIMNGIAAFGANYKNYGGTFLNFVSYAAGAVRLSALSGHPITWVATHDSIGLGEDGPTHQPIETLAHFRALPNCSVWRPADGNEVSAAYKSAVSSTSTPHILALSRQNLPQLEGSSIEKASKGGYVLKEASKPDVILVASGSEVEIAVNAAKLLEAEGKKVSVVSIPDFLTFDKQSDEYRLSVLPDGAPVVSIEVMSPFGWSKYSHEQFGLSRFGASGKAEDVYKYLEFTPEGVAERASKTITFYKGKEVLSPLTRAF

SEQ ID NO: 14 - protein, 678 aa, Debaryomyces sp.
  REGION 1..678: note = Transketolase from Debaryomyces
MSQDLDQLAISTIRLLSVDSVDKAQSGHPGAPLGLAPAAHAVWQKMKFNPKDTNWINRDRFILSNGHACALLYSLLVLYGYDLTVEDLKQFRQLNSKTPGHPEALDVPGCEVTTGPLGQGISNAVGLAMAQKQFGATYNKPDHKISDSYTYAFLGDGCLMEGVASETCSLAGHLQLNNLIAFWDDNHISIDGNTNVAFTEDVMLRFKAYGWHLIEIAEGDTDLDAIAKAIDEAKKVEDKPTLIRLTTTIGFGSLAQGTHGVHGAPLKADDIKQLKESMGFNPEETFVIPKEVTDYLKKHVEENQKVQEDWKKLFESYKQKYPELGAELQRRLDGKLPENWSDALPTYSPSDSAVATRKLSETVLTKILPVIPEIIGGSADLTGSNLTRAAGSVDFQPPATGLGDYSGRYIRYGVREHGMGAIMNGIAAFGANYKNYGGTFLNFVSYGAGAVRLSALSHHPVIWVATHDSIGLGEDGPTHQPIETLAHLRAIPNLSVWRPADGNETSAAYLQAIESTSTPSIIALTRQNLPQLEGSSIEKAAKGGYTLVKQDKPDVIIVSSGSEVSISADAAKKLESEGFKAGVVSMPDFLTFDKQPLDYRLSVLPDGVPIMSVEVMSSFGWSKYSHEQFGLNRFGASGKGPEIYKFFEFTPEGVADRAAKTIQFYKGKPLLSPLNRAF

SEQ ID NO: 15 - protein, 685 aa, Kuraishia capsulata
  REGION 1..685: note = Transketolase from Kuraishia capsulata CBS 1993
MSALDIKSINTIRVLAVDTVFKANSGHPGAPMGLAPAAHVLFQQMKFNPKNPDWINRDRFVLSNGHAVALLYTLLHLYGYSLTLDDLKSFRQINSKTPGHPEADIPGVEVTTGPLGQGIANAVGLAIAQAQFAATYNKPDFKISDNYTYAIFGDGCMMEGVASEACSLAGHLQLGNLIAFYDDNHISIDGDTNSAFTEDVTKRLESYGWEVLWVHNGNEDLAAIAAALEQAKKSSKPTCIRLTTTIGYGSLLQGSHSVHGSPLKADDIKQLKEKWGFDPSKTFDVPQEVYDSYAKTAAAGAAAEAEWNELFSKYQAAYPELGAELARRLKGELPAGWEKNLPTYTPADPAVASRKLSETVLTKIQAALPELVGGSADLTGSNLTRWPSAVDFQPPSTGLGEWSGRYFRFGVREHGMGAIMNGISAFGANYKAFGGTFLNFVSYAAGAVRLSALSGHPIIWVATHDSIGLGEDGPTHQPIETLAHFRAIPNLAVWRPADGNETSAAYYQALTSTHTPSIIALSRQNLPQLEGSSIEKASKGAYTLVAADKPDVIIAATGSEVYIAVDSVKVLAAQGIKASVVSVPDFHTFDRQTEEYQLSVFPDGVPILSVEVMSTAVWSKYSHEQFGINRFGISGKAADIYEYFGFTPEGIAERATKTVEFYKGKELLSPLHKAFYSAKYEFHKY

SEQ ID NO: 16 - protein, 682 aa, Candida arabinofermentans
  REGION 1..682: note = Transketolase from Candida arabinofermentans NRRL YB-2248
MSSELELKAVNTIRLLSVDAVSKANSGHPGAPMGMAPAAHALWTQMKFNPKNPEWINRDRFVLSNGHACALLYSLLHLFGYPLTLDDLKSFRQLGSKCPGHPEYETPGIEVTTGPLGQGICNAIGLAMAQAQFAATFNKPGFTLSDNYTYCFFGDGCMMEGVASEAMSLAGHLQLGNLIAFYDDNHISIDGDTNVAFTEDVAARLESYGWDMLYVKDADTDLNAISDAIAQAKKTSKPTCIKITTTIGYGSLLQGSHSVHGAPLKADDVKQLKKKWGFDPEKTFEVPQEVYDLYAKVGEAGAAAEAEWNTLLEKYTVEYPELGAELKRRLAGELPETFIKDLPVYSVDDDAVASRKLSEIVLSKIEKNLPELVGGSADLTGSNLTRWGAAVDFQPPSTGLGEYSGRYFRFGVREHGMGAIMNGFSAFGANYKVYGGTFLNFVSYAAGALRLSALSGHPVIWIATHDSIGLGEDGPTHQPIETLAHFRALPNLMVWRPADGNETSAAYLKAIESKHTPSVIALSRQNLPQLAGSSIEKAAKGGYVVYEASGDAQLILVSTGSEVAISIDGAKKLAEAGIPTTVVSMPDFHTFDKQSLEYRKSILPDGIPIMSVEVMSTAVWAKYSHVQFGINRFGASGKAPEIYKYFEFTPEGVASRGKKTVEFFKGSKLLSPLNTPFDSVTM

SEQ ID NO: 17 - protein, 677 aa, Metschnikowia pulcherrima
  REGION 1..677: note = Transketolase from Metschnikowia pulcherrima
MSDIDQLAISTIRLLAVDAVAKANSGHPGAPLGLAPAAHAVWKEMKFNPKNPDWVNRDRFVLSNGHACALLYAMLHLYGFDMSLDDLKQFRQLNSKTPGHPEKFEIPGAEVTTGPLGQGISNAVGLAIAQKQFAATFNKDDFAISDSYTYAFLGDGCLMEGVASEASSLAGHLQLNNLIAFWDDNKISIDGSTEVAFTEDVLKRYEAYGWDTLTIEKGDTDLEGVAQAIKTAKALKKPTLIRLTTIIGYGSLQQGTHGVHGAPLKPDDIKQLKEKFGFDPTKSFVVPQEVYDYYGTLVKKNQELESEWNKTVESYIQKFPEEGAVLARRLKGELPEDWAKCLPTYTADDKPLATRKLSEMALTKILDVVPELIGGSADLTGSNLTRAPDMVDFQPPQTGLGNYAGRYIRYGVREHGMGAIMNGIAGFGAGFRNYGGTFLNFVSYAAGAVRLSALSHLPVIWVATHDSIGLGEDGPTHQPIETLAHFRATPNISVWRPADGNEVSAAYKSAIESTSTPHILALTRQNLPQLAGSSVEKASTGGYTVYQTTDKPAVIIVASGSEVAISIDAAKKLEGEGIKANVVSLVDFHTFDKQPLDYRLSVLPDGVPIMSVEVMSSFGWSKYSHEQFGLNRFGASGKAEDLYKFFDFTPEGVADRAAKTVQFYKGKDLLSPLNRAF

SEQ ID NO: 18 - protein, 677 aa, Meyerozyma sp.
  REGION 1..677: note = Transketolase from Meyerozyma
MTDIDQLAIATIRLLSVDQVDAAQSGHPGAPLGLAPAAHAVFKKMKFNPKNTSWINRDRFILSNGHACALLYSMLCLYGYALNVDDLKRFRQVGSKTPGHPERLDVPGIEVTTGPLGQGISNAVGLALAQRQFAATYNKPDFPISDSYVYAFLGDGCLMEGVASEASSVAGHLQLGNLIAFWDDNKITIDGDTAVAFTEDVEARYRAYGWHTIHIKEGDSDLEGIAKAIEEAKKVTDKPTLIRLTTIIGFGSLQQGTHSVHGAALKADDIKQLKTKFGFNPDEKFVIPPEVTADYAKHVAENQRIEGEWNKLVADYKQKYPTEGAELARRLEGKLPDGWKDALPTYTPKDKTQATRALSQTVLEKLYPVIPELIGGSADLTGSNLTRATEAVDFQPPSSKLGDYSGRYIRYGVREHGMGAIMNGIAAFGANYKNFGGTFLNFVSYAAGAVRLSALSHHPVVWVATHDSIGLGEDGPTHQPVETLAHFRALPNMSVWRPADGNEVSAAYIAALETDSHPSIIALSRQGCPHLEGSSIEKALKGGYTVYEVEKPDVIIVSTGSEVNIAIEGAKQLNNDGIKANVVSMPDFFTFDQQPLSYQLSVLPDGVPIMSFEVMSTFGWSKYSHEHFGMNRFGMSGKGPEIYKALGFTPEGVADKAAKTVQFYKNKPVVSPLNKAI

SEQ ID NO: 19 - protein, 675 aa, Diutina rugosa
  REGION 1..675: note = Transketolase from Diutina rugosa
MSIEEKFINTIRLLAVDEVSKANSGHPGAPLGLAPTAHVIFKNMKFNPKEPNWPNRDRFVLSNGHACALLYAMLCLYGYKTTVDDLKQFRQVNSKTPGHPEADIDGVEVTTGPLGQGISNAVGLAIAQKQFAARFNTDKYTVSDNYTYCILGDGCLMEGVSSEASSLAGHLQLDNLIAFWDDNHITIDGDTNVAFTENVLERYKAYGWNVLHVANGDDDIDAIQKAIDEAKQTKGKPTLIKVTTVIGYGSLLQGTHGVHGAPLKADDVAQLKKKFGFDPEQSFVIPAEVEEFAAKHVTKNLKVYNDWINLVKNYVAEEKQGAELQRRIDGKLPADWTKALPTYTSSDKALATRKLSEMVLDNITAELPELIGGSADLTGSNLTKAKGTIDFQPPSTGLGDYDGVYIRYGVREHGMGAIMNGIHAFGAGYKAFGGTFLNFVSYASGAVRLSALSEFPVIWVATHDSIGLGEDGPTHQPIETLAHFRATPNLMVWRPADGNEVSAAYQAAIESTHTPSIIALTRQNLPQLEHSSIENALKGGYVVYPVENPDLIIVSSGSEVSLSIDTAKKMAGEGYKVRVVSMAEFLTFDKQPEDYKLSVLPDGVPIMSLEVMSTFGWSKYSHEQFGLNTFGASGKGPDVYKKFGFTVDGVSDKAAKVIEFYQGKQLVSPLKRAFN

SEQ ID NO: 20 - protein, 682 aa, Pachysolen tannophilus
  REGION 1..682: note = Transketolase from Pachysolen tannophilus NRRL Y-2460
MSDLDQLAINTIRLLAVDAVAKANSGHPGAPMGLAPAAHVLFQQMRFNPKDPAWLNRDRFVLSNGHACALLYSLLHLFGYDLGIEDLKQFRQLHSKTPGHPERGIPGVEVTTGPLGQGVGNAVGIAIAQKHVAAKFNKPQFSLIDGYTYCFFGDGCMMEGVASEAASLAGHLQLGNLICFYDDNHISIDGDTKFAFTEDVGKRLEAYGWHVITVKDANTDLAGIAAAIEEAKKVTDKPSCIRLVTTIGFGSLQAGTGSVHGSPLKPDDIIQVKKKFGFDPEKTFYVPKEVYELYAKTSKAGAAAEQEWQQLFAKYQQQYPELGEELARRLTDKLPQGWEKALPRYTPQDKALATRKLSEMVLTSISDIVPELIGGSADLTPSNLTRVSNAVDFQPPSLKIGDYSGRYIRFGVREHGMGAIMNGIAAFGSNLRPFGGTFLNFVSYAAGALRLAALSHHPVIWVATHDSIGLGEDGPTHQPIETLAHFRAIPNLMVWRPADGNEVSAAYYKSLSSEKTPSVIALSRQNLPQLEGSSIEKAAKGGYTLVSTDKPDVILVASGSEVSISVEAAKVLANKGIKANVVSIPDFFTFDRQPRDYQLSVLPNGVPSLSVEVMSTAGWSKYSHEQFGINTFGASGKAADIYKSFEFTPEGIADRAEKTYNFYKGHELLSPLNKAFESIHYF

SEQ ID NO: 21 - protein, 692 aa, Rasamsonia emersonii
  REGION 1..692: note = Transketolase from Rasamsonia emersonii CBS 393.64
MGYTELDQLAINTIRVLATNNSGVTQVDATSKANAGHPGAPMGLAPVAHVLFNKFMNFNPKNPDWVNRDRFVLSNGHGCMLQYALLHLFGYNVSIEDLKNFRQIGSITPGHPEAKETPGVEVTTGPLGQGFSNAVGLAIAQAHTAAVFNKPGYELFNNYTYTIFGDGCAMEGVACEAASMAGHLKLGNLIAIYDDNHISIDGDTKVAFTEDVLKRFEAYGWHTQWVKDGDHDLQAIEDAIRKAQEVKDKPSIIKLTTTIGYGSKLQGTAGVHGNPLKADDAQSVKRKFGFNPDQSFVVPQEVYDLYGKRAAEGAAKEQEWNALLEKYATEYKAEYDDLLRRLSGKLPEGWEKNLPTYKPTDAAVASRKLSETVLEKIHGVIPELLSGSADLTGSNNTRWKNAVDFQPPEYGIGDWSGRYLRYGVREHAMGAVMNGLAAYGTVIPAGGTFLNFVSYAAGAVRLSALSRVRVIWIATHDSIGLGEDGPTHQPIETLVHFRATPNLMVWRPADGNETSAAYYSALTAKNTPSILALTRQNLPQLQNSTIEAALKGGYVILEAPDAAITIVSTGSEVSLSIDAANYLKEKHGIVARVVSLPCWEVFDAQDKAYRLQVIPDGIPVLSVEAASTIGWERYSHEQFGLNRFGASGPYKELYKKFELTPEGISKRAIATIEYYKGQKVRSPINRAFEQVV

SEQ ID NO: 22 - protein, 679 aa, Kluyveromyces marxianus
  REGION 1..679: note = Transketolase from Kluyveromyces marxianus (strain DMKU3-1042 /BCC 29191 / NBRC 104275)
MSQYSDIDRLAINTIRLLSVDQVASANSGHPGAPLGLAPAAHVVWKQMRLNPKNPEWINRDRFVLSNGHACALLYSLLHLFGYDFSIEDLKQFRHLGSKTPGHPEFELPGVEVTTGPLGQGISNAVGLAIAQANFAATYNKPDFELSDSFTYVFLGDGCLQEGVSSEACSLAGHLKLKNLIAFYDDNKITIDGNTNVSFDEDVGKRYEAYGWEVLSVENGNDDLDSISKALEQAKRSDKPTLIKLVTTIGFGSLQAGTHAVHGAPLKADDIKQLKTKFGFNPEESFVVPQEVYDLYNKSTIEPGIEANNKWNALFEAYSAKFPELGAEIKRRLAGELPTGWENKLPTYTPKDSAVASRKLSELLLQNIFEDIPELIGGSADLTPSNLTRTKEVVDFQAPSSGLGDYTGRYIRYGVREHGMGAIMNGISAFGANYKPYGGTFLNFVSYAAGALRLSALSGHPVIWVATHDSIGLGEDGPTHQPIETLAHFRALPNLQVWRPADGNEVSAAYKVALKSKHTPAVIALSRQNLPQLEGSSIEKAAKGGYVLQDVEQPDIAIVSTGSEVGIAVEAAKVLAEKNIKARIVSLPDFHTFDQQPKEYQLSVLPDGVPILSVEVLSTSGWAKYSHQQFGLNRFGASGKGPAVYEKFDFTPQGIASRAEKTVEFYKGKQVYSPLNTAF

SEQ ID NO: 23 - protein, 676 aa, Ogataea parapolymorpha
  REGION 1..676: note = Transketolase from Ogataea parapolymorpha DL-1
MSQDIQTKAINTIRVLAADIVAKANSGHPGAPMGMAPAAHAVYTQMKFNPKNPHWINRDRFVLSNGHACALLYTLLHLFGYDLTMDDLKSFRQINSKTPGHPEVGIPGVEVTTGPLGQGISNAVGLAIAQANFAATYNKPGYTLSDNYTYCFFGDGCMMEGVASEAMSLAGHLQLGNLIAFYDDNKISIDGSTEVAFTEDVCGRLEKYGWDIFEVPDADTDVAAISEAIAKAKKTNKPSCIRIRTTIGYGSLNAGSHSVHGAPLKKDDIIQLKEKWGFDPEKSFVVPQEVYDYYHKISEAGAAAEAEWNKLFEAYQKEFPKEGAELARRLRGELPEGWQNVLPTYQPGDAAVASRKLSEVCLGKLQAVLPELVGGSADLTPSNLTRWSGAIDFQPESTGLGDYSGKYLRFGVREHGMGAIINGISAYGANYKSFGATFLNFVSYASGALRLSALSHHPVIWVATHDSIGLGEDGPTHQPIETLAHFRAIPNLMVWRPADGNETSAAYIKAISSTKTPSVLALSRQNLPQLPGSSIEKALKGGYTVHEVDNAKLILVATGSEVSLSIDAAKLLTEKGIPTAVVSIPDFFTFDQQPADYKLSVLPDGVPILSVEVMATSGWAKYAHAQFGLNRFGASGKTADVYKFFDFTPEGVAKRGEQTYEFFKGKNLISPLHTPF

SEQ ID NO: 24 - protein, 681 aa, synthetic construct
  REGION 1..681: note = Transketolase, synthetic, based on Komagataella phaffii
MSDLLAINTIRLLAVDTVEQYGGGHPGAPMGLAPAAHTLFKQMRFNPRNPAWINRDRFVLSNGHACALLYTMLFLYGYDYTIDDLQSYHSSDYHSLTPGHPEAELPGVEVTTGPLGQGIANAVGLAIAQAQLAATYNKPNYELFSNYTYAFLGDGCLQEGVAQEAISLAGHLGDGLKLIAFWDDNQISIDGDTNVSFTEDVPAKFRAQGWEVLSVKDGNDDLEGIAAALAKAKTTNKPTLIRLPTIIGYGSLQQGTHGVHGSPLKPDDIKQLKKKFGFDPEQNFVVPREVTESYAKHVADNQQVEVEWNKLLTAYTKEYPELGQELHRRLDGKLPENWQKALPTYTVDDKPVASRKLSEIVLTSIEKELPELVGGSADLTGSNLTRWPDAVDFQPKSTGLGDFSGRYFRFGVREHGMGAIINGISAYGANFKAYGGTFLNFVSYASGAVRLSALSGHPIIWVATHDSIGLGEDGPTHQPIETLAHLRALPNLMVWRPADGNETSAAYLRAIESKHTPSIIALTRQNLPQLEHSSIEKAAKGGYTVYPVENPDIILVASGSEVSIAIDGAKKLGTEGVKASVVSIPDFFTFDSQARSYQLSVLPDGVPIMSVEVMSTFGWSKYSHEQFGINRFGISGPGPEIYKFFEFTAEGVADRASKVVQFYKGKELLSPLNKAFESVHA

SEQ ID NO: 25 - protein, 679 aa, Komagataella phaffii
  REGION 1..679: note = Transketolase 1 (TKL1) from Komagataella phaffii
MSDLLAINTIRLLAVDTVAKANSGHPGAPMGLAPAAHTLFKQMRFNPRNPAWINRDRFVLSNGHACALLYTMLFLYGYDYTIDDLKSFRQLNSKTPGHPEAELPGVEVTTGPLGQGIANAVGLAIAQAQLAATYNKPNYELFSNYTYAFLGDGCLQEGVAQEAISLAGHLGDGLKLIAFWDDNQISIDGDTNVSFTEDVPAKFRAQGWEVLSVKDGNDDLEGIAAALAKAKTTNKPTLIRLPTIIGYGSLQQGTHGVHGSPLKPDDIKQLKKKFGFDPEQNFVVPREVTESYAKHVADNQQVEVEWNKLLTAYTKEYPELGQELHRRLDGKLPENWQKALPTYTVDDKPVASRKLSEIVLTSIEKELPELVGGSADLTGSNLTRWPDAVDFQPKSTGLGDFSGRYFRFGVREHGMGAIINGISAYGANFKAYGGTFLNFVSYASGAVRLSALSGHPIIWVATHDSIGLGEDGPTHQPIETLAHLRALPNLMVWRPADGNETSAAYLRAIESKHTPSIIALTRQNLPQLEHSSIEKAAKGGYTVYPVENPDIILVASGSEVSIAIDGAKKLGTEGVKASVVSIPDFFTFDSQARSYQLSVLPDGVPIMSVEVMSTFGWSKYSHEQFGINRFGISGPGPEIYKFFEFTAEGVADRASKVVQFYKGKELLSPLNKAFESVHA

SEQ ID NO: 26 - DNA, 2056 bp, Komagataella phaffii
  misc_feature 1..2056: note = Transketolase 1 (TKL1)
  source 1..2056: mol_type = unassigned DNA
ggtctcgaatgtctgatttgttggccatcaacaccatcagattgttggctgttgacactgttgccaaggctaactctggtcacccaggtgctccaatgggtttggctccagctgctcacactttgttcaagcaaatgagattcaacccaagaaacccagcttggatcaacagagacagatttgttttgtccaatggtcacgcttgtgctttgctatacaccatgttgttcttgtacggttacgactacaccattgatgacttgaaatccttccgtcaattgaactctaagactccaggtcatccagaagctgaattaccaggtgttgaagtcactaccggtccattgggtcaaggtattgctaacgctgtcggtttggctattgctcaagctcaattggctgctacctacaacaagcctaactacgaattattctccaactacacttacgctttcttaggtgacggttgtttgcaagaaggtgttgctcaagaagccatctccttggctggtcacttgggtgatggtttgaaattaattgctttctgggacgacaaccaaatctctattgacggtgacactaacgtctccttcactgaagatgttccagctaagttcagagcccaaggttgggaagtcttgtccgtcaaggacggtaacgacgacttggaaggtatcgctgccgctttggctaaggccaagactaccaacaagccaactttgatcagattgcctaccatcatcggttacggttctttgcaacaaggtactcacggtgttcacggttctccattgaagccagatgatatcaagcaattgaagaagaagtttggtttcgacccagaacaaaactttgttgttccaagagaagttaccgaatcttacgccaagcatgttgctgacaaccaacaagtcgaagttgaatggaacaaattgttgactgcttacaccaaggaatacccagaattgggccaagaattgcacagaagattggacggtaagttgccagaaaactggcaaaaggccttgccaacctacaccgtcgatgacaagccagttgcctctcgtaaattatctgaaatcgtcttaacttccattgaaaaggaattgccagaattagtcggtggttctgctgacttgaccggttccaacttgactagatggccagatgctgtcgacttccaaccaaagtctaccggtttaggtgacttctccggtcgttatttcagatttggtgttagagaacacggtatgggtgccattattaacggtatctctgcctacggtgctaacttcaaggcttacggtggtactttcttgaactttgtttcctacgcttctggtgctgttagattgtccgctttgtccggtcatccaatcatctgggttgctactcacgattccatcggtttgggtgaagacggtccaactcaccaaccaatcgaaactttggctcacttaagagctttgccaaacttgatggtctggcgtccagctgatggtaatgaaacctctgctgcctacttgagagccattgaatccaagcacactccatctatcatcgctttgaccagacaaaacttgcctcaattggaacactcttctatcgaaaaagctgccaagggtggttacaccgtctacccagttgaaaaccctgatattattttggttgcttctggttctgaagtttccattgccattgatggtgccaagaagttgggtaccgaaggtgtcaaggcttctgttgtctccatcccagatttcttcaccttcgactctcaagctagatcttaccaattgtctgttttgccagatggtgtcccaatcatgtccgtcgaagttatgtctactttcggttggtctaagtactcccacgaacaattcggtattaacagattcggaatttctggtccaggtccagaaatctacaaattcttcgaattcaccgctgaaggtgtcgctgacagagcttctaaggttgtccaattctacaagggtaaggaactattatctccattaaacaaggctttcgaatccgtccacgcgtaaaggagacc

SEQ ID NO: 27 - protein, 679 aa, Kluyveromyces lactis
  REGION 1..679: note = Transketolase from Kluyveromyces lactis
MSQYTDIDRLAVSTIRLLAVDQVSAANSGHPGAPLGLAPAAHVIWKQMRLNPKNPEWINRDRFVLSNGHACALLYSLLHLFGYDMSIEDLKHFRHLGSKTPGHPEFELPGVEVTTGPLGQGISNAVGMAIAQANFAATYNKPDYELSDSYTYVFLGDGCLQEGVSSEASSLAGHLRLKNLIAFYDDNQITIDGNINVSFDEDVSKRYEAYGWEVLHVENGNDDLDAISKALEQAKLSDRPTLIKLTTTIGFGSLNAGSHSVHGAPLKADDVKQLKVKFGFNPEESFVVPQEVYDLYNKSTIEPGIEANKQWDALLDAYVGQFPELGAEVKRRLAGEFPEGWESKLPTYTPEDSAVASRKLSEIVLDNVFDTLPELLGGSADLTPSNLTRSKGAVDFQPPITGLGDYSGRYIRYGVREHGMGAIMNGISAFGANYRPYGGTFLNFVSYASGAVRLSALSGHPVIWVATHDSIGLGEDGPTHQPIETLAHFRAIPNLQVWRPADGNEVTAAYKVALTNKHTPAIIALSRQNLPQLQGSSVEKAVKGGYILQDVDQPDLAIVSTGSEVGIAVEAAKVLAEKNIKARIVSLPDFHSFGQQSKEYQLSVFPDGVPILSVEVLATSGWSKYAHQSFGLDRFGASGKGPAVYEKFEFTPQGIATRAEKTVEFYKGKQVISPLNTAF

SEQ ID NO: 28 - DNA, 2056 bp, Kluyveromyces lactis
  misc_feature 1..2056: note = Transketolase from Kluyveromyces lactis
  source 1..2056: mol_type = unassigned DNA
ggtctcgaatgtctcaatacactgatatcgatcgtttggctgtctctactatcagattgttggctgtcgaccaagtctctgctgctaactctggtcaccctggtgccccattgggtttggctccagctgctcacgtcatctggaaacaaatgagattaaacccaaagaacccagaatggatcaacagagacagattcgttttgtccaacggtcacgcttgtgctctattatactctttgttgcatttgttcggttacgacatgtctatcgaagatttgaagcacttcagacatttgggttccaagaccccaggtcacccagaatttgaattgccaggtgttgaagttaccactggtccattgggtcaaggtatctccaatgctgttggtatggctattgcccaagctaacttcgccgctacttacaacaagccagactacgaattgtctgattcttacacctacgtcttcttgggtgacggttgtttgcaagaaggtgtttcctctgaagcttcttctttagctggtcatttgagattgaagaacttgattgctttctacgacgacaaccaaatcaccattgatggtaacatcaacgtctctttcgatgaagatgtttccaagagatacgaagcctacggttgggaagtcttgcacgttgaaaacggtaatgatgatttggatgctatctccaaggctttggaacaagccaagttgtctgacagacctaccttgatcaaattgaccaccaccatcggtttcggttctttgaacgctggttctcactctgttcacggtgctccattaaaggccgatgacgttaagcaattgaaggtcaaattcggtttcaacccagaagaatctttcgttgttcctcaagaagtctacgacttgtacaacaagtctactattgaaccaggtatcgaagctaacaagcaatgggatgccttgttggacgcttacgttggtcaattcccagaattgggtgctgaagtcaagagaagattggccggtgaatttccagaaggttgggaatctaagttgccaacttacactccagaagattctgccgtcgcttccagaaagttgtccgaaattgtcttggacaacgttttcgacactttgccagaattgctaggtggttctgctgacttgactccatccaacttaaccagatccaagggtgctgtcgatttccaaccaccaatcactggtttgggtgactactccggtcgttacatcagatacggtgttagagaacacggtatgggtgctatcatgaacggtatctctgccttcggtgctaactacagaccatacggtggtactttcttgaacttcgtttcttacgcttctggtgctgttagattatccgctttgtctggtcatccagttatctgggtcgccactcacgactctattggtctaggtgaagacggtccaactcaccaaccaatcgaaaccttagcccacttcagagctatcccaaacttacaagtttggcgtccagccgacggtaacgaagtcactgctgcttacaaggttgctttgaccaacaagcacactccagctatcattgctttatctagacaaaacttgcctcaattgcaaggttcctccgtcgaaaaggctgtcaaaggtggttacattttgcaagacgtcgaccaaccagacttggctatcgtttccaccggttctgaagttggtattgctgtcgaagctgccaaggttttggccgaaaagaacattaaggctagaattgtttctttaccagacttccactcctttggtcaacaatccaaggaataccaattgtctgttttcccagacggtgtcccaattttgtccgttgaagttttggctacctccggttggtccaaatacgctcaccaatctttcggtttggatcgtttcggtgcttccggtaaaggtccagctgtctacgaaaagttcgaattcaccccacaaggtattgctaccagagctgaaaagaccgttgaattctacaagggtaagcaagtcatttctccattgaacactgccttttaaaggagacc

SEQ ID NO: 29 - skipped

SEQ ID NO: 30 - DNA, 12 bp, synthetic construct
  misc_feature 1..12: note = Promoter motif
  source 1..12: mol_type = other DNA
aaaaattgttga

SEQ ID NO: 31 - DNA, 622 bp, Saccharomyces cerevisiae
  misc_feature 1..622: note = Promoter ANB1 from Saccharomyces cerevisiae
  source 1..622: mol_type = genomic DNA
ggtctcggtgcagatcggtgaaaacatcttgatcttgctcccgggaattttagattcaggtaggaaattgattacatcaatactgttaccctgaatcatattcgacgatgtcgtctcacacggaaatataattcatttcttggttttccaaaaaaattttcattttttttcacttttttgtttcgtcctcctttttttttttttttttttattttttttcctgtgttcacctttttttttttcagttgacatctttctgcattcttttctgtgtttttttttttttttttcgtttttccattgttcgttcgttgcctgttttttcgccctattgttctcgagcctaaaaattttttcctttcctgctttcctttcttcgttcaaagtttcctattccattgttctctttggtaaactcattgttgtcggaactcagatatattcaggtcaatttactgtacttcaattgacttttttcttgaaatttcaacttgccttttcaacttgttcttcttttttaatcttattctacactttagttcccttaccttgttcctaattattgtctagcaaaaagaaaacatacacctatttcattcacacactaaaacaatgggagacc

SEQ ID NO: 32 - DNA, 825 bp, Saccharomyces cerevisiae
  misc_feature 1..825: note = Promoter DAN1 from Saccharomyces cerevisiae
  source 1..825: mol_type = unassigned DNA
agaatgcaagggcaaacaggcctgaggtaactttaaaatataatgtaccttgaaaactatgtatacattgcatttattaagcagtagggtattttgtttgcaagaaaataaagctcaaaatatctttggagtttgacaatagtatagaaaaaatagctcagcaaccttaataaaaaaagggtcattgggcaaagcgttttaaaaagatttaagtggtagtgtttaaagtgaaagagattgatatagttaaaaattgttgagctcaattcacgctggattcggcgatccgttttcttcaatcctcacgtgctttcttcgtttgagtgcaaaagttcatatgatgctatctcccgcttatcttattagtcgaaaatggggagaatttcctattttatctgtcgtttagcacatacggccaggaaaatacataagatttcgccgaacgacggggtcaattcgtcctttttgtacacatcgtttaatttatgaggaaaaattgatgaacgtatcctccgtagacgctcctctgaaaagtttcatgtttcctgcgcgttcctttgataggcaataaaacaatacaacgcgtgcctttgaaaatgccaagatctatacgaggcctctaacaaaacatcgttcaggaacagagaatgctaaaaatgcaaaagggtccctgggtactcattgaatagaaatgattgaaaatactgcgtataaaatagcacgactaaatgatactatttttatgtcgacacggtactatttcttcttttgcagataaaagtgtagcagataaaagtgtagcatactaaatatataccccaagta

SEQ ID NO: 33 - protein, 365 aa, Klebsiella pneumoniae
  REGION 1..365: note = K. pneumoniae glycerol dehydrogenase (Kpne_gldA)
MLKVIQSPAKYLQGPDAAVLFGQYAKNLAESFFVIADDFVMKLAGEKVVNGLQSHDIRCHAERFNGECSHAEINRLMAILQKQGCRGVVGIGGGKTLDTAKAIGYYQKLPVVVIPTIASTDAPTSALSVIYTEAGEFEEYLIYPKNPDMVVMDTAIIAKAPVRLLVSGMGDALSTWFEAKACYDARATSMAGGQSTEAALSLARLCYDTLLAEGEKARLAAQAGVVTEALERIIEANTYLSGIGFESSGLAAAHAIHNGFTILEECHHLYHGEKVAFGTLAQLVLQNSPMDEIETVLGFCQRVGLPVTLAQMGVKEGIDAKIAAVAKATCAEGETIHNMPFAVTPESVHAAILTADLLGQQWLAR

SEQ ID NO: 34 - protein, 367 aa, Enterococcus aerogenes
  REGION 1..367: note = E. aerogenes glycerol dehydrogenase (Eaer_gldA)
MDRIIQSPGKYIQGAGAIKRLGEYLKPLAERWLIIGDKFVLGFAEEQLRTSLGGAGLVAEIAPFGGECSQNEINRLRDIASSAQCHAVLGIGGGKTLDTAKALAHYMHLPVVVAPTIASTDAPCSALSVIYTDDGEFESYLMLPHNPNMVVVDTQIVAAAPARLLAAGIGDALATWFEARACSRSGATTMAGGKCTQAALALAELCYNTLVEEGEKAMLAAEQHVVTPALERVIEANTYLSGVGFESGGLAAAHAIHNGLTAIPDAHHFYHGEKVAFGTLTQLVLENAPVEEIETAAALCHSVGLPITLAQLDIKGDIPAKMRTVAEAACAEGETIHNMPGGACADQVYAALLVADQYGQRFLQEWE

SEQ ID NO: 35 - protein, 364 aa, Yersinia aldovae
  REGION 1..364: note = Y. aldovae glycerol dehydrogenase
MLKVIQSPSKYIQGANALQSIGEFAKLLANNYFIIADDFVMKLTADTVGTSLQTCELKSHFSRFNGECSRQEIERLTVELKKYGCNGVIGIGGGKTLDTAKAIAHYQHIPVVVVPTIASTDAPTSALSVIYTEQGEFAEYLIYPKNPDIVLMDTTIIAKAPVRLLVAGMGDALSTYFEAQACFDAKAISMAGGASTLAAITLARLCYETLLAEGYKAKLAVEAGVVTEAVERIIEANTYLSGIGFESSGLAAAHAIHNGFTVLEECHHLYHGEKVAFGTLTQLVLQNSSMEEIETVLSFCQQLGLPITLAEMGVTQDLECKIRAVAQASCAEGETIHNMPFKVTADSVYAAIIVADRLGQAFLN

SEQ ID NO: 36 - protein, 367 aa, Escherichia coli
  REGION 1..367: note = E. coli glycerol dehydrogenase (Ec_gldA)
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 37 - DNA, 1111 bp, synthetic construct
  misc_feature 1..1111: note = nucleotide seqeunce encoding E.coli gldA codon-optimised
  source 1..1111: mol_type = other DNA
atggacagaatcatccaatctccaggtaagtacatccaaggtgctgacgttatcaacagattgggtgaatacttgaagccattggctgaaagatggttggttgttggtgacaagttcgttttgggtttcgctcaatctaccgttgaaaagtctttcaaggacgctggtttggttgttgaaatcgctccattcggtggtgaatgttctcaaaacgaaatcgacagattgagaggtatcgctgaaaccgctcaatgtggtgctatcttgggtatcggtggtggtaagaccttggacaccgctaaggctttggctcacttcatgggtgttccagttgctatcgctccaaccatcgcttctaccgacgctccatgttctgctttgtctgttatctacaccgacgaaggtgaattcgacagatacttgttgttgccaaacaacccaaacatggttatcgttgacaccaagatcgttgctggtgctccagctagattgttggcagctggtatcggtgacgctttggctacctggttcgaagctagagcttgttctagatctggtgctaccaccatggctggtggtaagtgtacccaagctgctttggctttggctgaattgtgttacaacaccttgttggaagaaggtgaaaaggctatgttggctgctgaacaacacgttgttaccccagctttggaaagagttatcgaagctaacacctacttgtctggtgttggtttcgaatctggtggtttggctgctgctcacgctgttcacaacggtttgaccgctatcccagacgctcaccactactaccacggtgaaaaggttgctttcggtaccttgacccaattggttttggaaaacgctccagttgaagaaatcgaaaccgttgctgctttgtctcacgctgttggtttgccaatcaccttggctcaattggacatcaaggaagacgttccagctaagatgagaatcgttgctgaagctgcttgtgctgaaggtgaaaccatccacaacatgccaggtggtgctaccccagaccaagtttacgctgctttgttggttgctgaccaatacggtcaaagattcctacaagaatgggaataaggcgcgc

SEQ ID NO: 38 - protein, 584 aa, Saccharomyces cerevisiae
  REGION 1..584: note = S. cerevisiae dihydroxyacetone kinase (S. cerevisiae DAK1)
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 39 - protein, 591 aa, Saccharomyces cerevisiae
  REGION 1..591: note = S. cerevisiae dihydroxyacetone kinase (S. cerevisiae DAK2)
MSHKQFKSDGNIVTPYLLGLARSNPGLTVIKHDRVVFRTASAPNSGNPPKVSLVSGGGSGHEPTHAGFVGEGALDAIAAGAIFASPSTKQIYSAIKAVESPKGTLIIVKNYTGDIIHFGLAAERAKAAGMKVELVAVGDDVSVGKKKGSLVGRRGLGATVLVHKIAGAAASHGLELAEVAEVAQSVVDNSVTIAASLDHCTVPGHKPEAILGENEYEIGMGIHNESGTYKSSPLPSISELVSQMLPLLLDEDEDRSYVKFEPKEDVVLMVNNMGGMSNLELGYAAEVISEQLIDKYQIVPKRTITGAFITALNGPGFGITLMNASKAGGDILKYFDYPTTASGWNQMYHSAKDWEVLAKGQVPTAPSLKTLRNEKGSGVKADYDTFAKILLAGIAKINEVEPKVTWYDTIAGDGDCGTTLVSGGEALEEAIKNHTLRLEDAALGIEDIAYMVEDSMGGTSGGLYSIYLSALAQGVRDSGDKELTAETFKKASNVALDALYKYTRARPGYRTLIDALQPFVEALKAGKGPRAAAQAAYDGAEKTRKMDALVGRASYVAKEELRKLDSEGGLPDPGAVGLAALLDGFVTAAGY

SEQ ID NO: 40 - protein, 572 aa, Klebsiella pneumoniae
  REGION 1..572: note = K. pneumoniae dihydroxyacetone kinase (Kpne_dhaK)
MTTKQFQFDSDPLNSALAATAEASGLAYLPKSKVIYYPLTNDKVTLISGGGAGHEPAQTGFVGPGLLDAAVSGQIFASPSTKQIIAGVNAVKSQRGSIIIVMNYTGDVIHFGMAAEQLRSRYDYHAELVSIGDDISVNKKAGRRGLAGTVLVHKIAGHLARDGWDVGVLAEALRTTAANLATVAASLEHCTVPGRKFETELAADEMEIGMGIHNEPGVKTIKIGKVESLLDELVDKFEPSKQDFVPFNKGDEVVLLVNSLGGVSSLELHAIANIAQTKFEKVLGVKTVRLIVGNFMAAFNGPGFSLTLLNVTTTAKKGNFDVLGALDAPVSTAAWPSLQQKDKPANGGVQEEKETDSDKPAEPTGIKADGKLFKAMIESAVDDLKKEEPQITKYDTIAGDGDCGETLLAGGDGILDAIKNKKIDLDDAAGVADISHIVENSMGGTSGGLYSIFFSGLVVGIKETKAKELSVDVFAKACETALETLSKYTQARVGDRTLMDALVPFVETLSKTKDFAKAVEAARKGADETSKLPANFGRASYVNEEGLENIPDPGALGLAVIFEGLLKAWEKK

SEQ ID NO: 41 - protein, 580 aa, Yarrowia lipolytica
  REGION 1..580: note = Y. lipolytica dihydroxyacetone kinase (Ylip_DAK1)
MDKHFINDPEVLVLDGLKSLADMNKTLTVHEEGKFIYFHDYNKKNVSVISGGGAGHEPTHSSFVGKGMLTAAVSGSIFASPSSKQIYTGIKQVESEAGTLVICKNYTGDILHFGMALEKQRTAGKKAELIAVADDVSVGRKKSGKVGRRGLSGTVLVHKIAGAAAARGLPLEAVTTIAKAAIDNLVSIGASLAHVHVPGHEPIAKEDEMKHDEMELGMGIHNEPGCKRISPIPSIDDLIAQMLKQMLDQSDKDRAYVKIEGDDEVVLLMNNLGGLSMLEFSAISHKVKEALAKEYKINPVRIFAGPFTTSLNGLGFGITLLRTTDRVKVEGEEYSLVDLIDQPVEAIGWPLCQPSDLKSKNKIGNVSIEEGQKDVKSPVTVDKEKVRQAIVNSMENLIKAEPKITKFDTMAGDGDCGTTLKRGAEGVLKFVKSDKFSDDPIRIVRDIADVIEDNMDGTSGALYAIFFHGFAKGMKDTLEKSKDISSKTWAAGLKVALDTLFKYTPARPGDSTMCDALVPFVETFVKTNDLNAAVEEARKGADATADMQAKLGRAVYVGDDVKVPDAGALGVVAIVEGFTK

SEQ ID NO: 42 - protein, 580 aa, Schizosaccharomyces pombe; identical to SEQ ID NO: 41
  REGION 1..580: note = S. pombe dihydroxyacetone kinase (Spom_DAK1)

SEQ ID NO: 43 - DNA, 1755 bp, Saccharomyces cerevisiae
  misc_feature 1..1755: note = S. cerevisiae dihydroxyacetone kinase (S. cerevisiae DAK1)
  source 1..1755: mol_type = genomic DNA
atgtccgctaaatcgtttgaagtcacagatccagtcaattcaagtctcaaagggtttgcccttgctaacccctccattacgctggtccctgaagaaaaaattctcttcagaaagaccgattccgacaagatcgcattaatttctggtggtggtagtggacatgaacctacacacgccggtttcattggtaagggtatgttgagtggcgccgtggttggcgaaatttttgcatccccttcaacaaaacagattttaaatgcaatccgtttagtcaatgaaaatgcgtctggcgttttattgattgtgaagaactacacaggtgatgttttgcattttggtctgtccgctgagagagcaagagccttgggtattaactgccgcgttgctgtcataggtgatgatgttgcagttggcagagaaaagggtggtatggttggtagaagagcattggcaggtaccgttttggttcataagattgtaggtgccttcgcagaagaatattctagtaagtatggcttagacggtacagctaaagtggctaaaattatcaacgacaatttggtgaccattggatcttctttagaccattgtaaagttcctggcaggaaattcgaaagtgaattaaacgaaaaacaaatggaattgggtatgggtattcataacgaacctggtgtgaaagttttagaccctattccttctaccgaagacttgatctccaagtatatgctaccaaaactattggatccaaacgataaggatagagcttttgtaaagtttgatgaagatgatgaagttgtcttgttagttaacaatctcggcggtgtttctaattttgttattagttctatcacttccaaaactacggatttcttaaaggaaaattacaacataaccccggttcaaacaattgctggcacattgatgacctccttcaatggtaatgggttcagtatcacattactaaacgccactaaggctacaaaggctttgcaatctgattttgaggagatcaaatcagtactagacttgttgaacgcatttacgaacgcaccgggctggccaattgcagattttgaaaagacttctgccccatctgttaacgatgacttgttacataatgaagtaacagcaaaggccgtcggtacctatgactttgacaagtttgctgagtggatgaagagtggtgctgaacaagttatcaagagcgaaccgcacattacggaactagacaatcaagttggtgatggtgattgtggttacactttagtggcaggagttaaaggcatcaccgaaaaccttgacaagctgtcgaaggactcattatctcaggcggttgcccaaatttcagatttcattgaaggctcaatgggaggtacttctggtggtttatattctattcttttgtcgggtttttcacacggattaattcaggtttgtaaatcaaaggatgaacccgtcactaaggaaattgtggctaagtcactcggaattgcattggatactttatacaaatatacaaaggcaaggaagggatcatccaccatgattgatgctttagaaccattcgttaaagaatttactgcatctaaggatttcaataaggcggtaaaagctgcagaggaaggtgctaaatccactgctacattcgaggccaaatttggcagagcttcgtatgtcggcgattcatctcaagtagaagatcctggtgcagtaggcctatgtgagtttttgaagggggttcaaagcgccttgtaa

SEQ ID NO: 44 - DNA, 1776 bp, Saccharomyces cerevisiae
  misc_feature 1..1776: note = S. cerevisiae dihydroxyacetone kinase (S. cerevisiae DAK2)
  source 1..1776: mol_type = genomic DNA
atgtctcacaaacaattcaaatcagatggaaacatcgttactccctacctacttggccttgctcgaagcaatcccggccttacagtgattaagcacgacagagtggttttccggactgcgtcagctcctaattcagggaaccctcctaaagtttcattggtttctggaggtggcagtggtcatgagccaacgcatgccggttttgttggtgaaggtgccttagatgcgattgcagcaggtgccatttttgcttctccttcaactaaacagatctattctgctattaaagctgttgaatctcctaagggtaccttgatcattgtaaaaaattacaccggtgatattatacattttggtctcgctgctgaaagagctaaagctgctggaatgaaagtcgaactggttgctgtaggagatgatgtctctgtcggtaagaagaaaggttctttagtcgggcgtcgaggtctcggagccaccgtattggtgcataaaattgctggggcagccgcttctcatggactggagttggcagaagttgccgaagttgctcagtcagtagttgacaatagtgtcacaattgcggcatctcttgatcactgcacggttcctggccacaaacctgaagccattttgggcgagaatgagtatgaaatcggtatgggtattcataacgagtctggtacctataagtcttctccgctgccatcgatttctgagctcgtttcccagatgcttcctcttcttctcgatgaggatgaagaccgttcttatgtgaagtttgagcccaaagaggacgtagttcttatggttaacaacatgggtggtatgtctaatctagaattgggttatgctgcagaggtcatttctgaacaattgattgataagtatcaaattgtgcccaagagaacgattactggagcattcattactgcattgaatggtcctgggtttggtatcactcttatgaacgcttcaaaagctggtggcgatatccttaagtatttcgattatcctaccacagcgagtggatggaatcaaatgtaccattctgccaaagattgggaggtacttgccaaagggcaggttcccaccgccccctctttaaagacattgaggaatgaaaaaggttcgggtgtgaaagctgattatgacacttttgctaaaattttgcttgctgggattgcaaaaattaacgaggttgaaccaaaggttacttggtacgataccattgcaggagatggtgattgcggaactactcttgtgtccggtggtgaagcattggaagaagctattaaaaaccatacgttgcgcctcgaggatgctgctcttggtatcgaagatattgcgtatatggttgaggattctatgggtggtacgtccggtggtctgtactctatctatctttctgctctcgcacaaggagttagggattctggggacaaggaacttactgcggaaactttcaaaaaggcatcaaacgttgcactagatgctttgtataagtatacgagagcccgtcctggttacaggactctgatcgatgctctgcaaccttttgtcgaagcgctgaaagccgggaagggtcccagagccgccgcccaagctgcttatgatggtgccgaaaagacaaggaagatggatgcccttgttgggcgtgcttcttacgtggctaaggaggagctgagaaaactcgacagcgaaggtggattaccagatccaggagcagttggtcttgctgcactactcgatggatttgttacagctgctgggtactag

SEQ ID NO: 45 - protein, 291 aa, Danio rerio
  REGION 1..291: note = D. rerio aquaporin 9 (Drer_T3)
MEYLENIRNLRGRCVLRRDIIREFLAELLGTFVLILFGCGSVAQTVLSREAKGQLLTIHFGFTLGVMLAVYMAGGVSGGHVNPAVSLAMVVLRKLPLKKFPVYVLAQFLGAFFGSCAVYCLYYDAFTEFANGELAVTGPNVTAGIFASYPREGLSLLNGFIDQVIGAGALVLCILAVVDKKNIGAPKGMEPLLVGLSILAIGVSMALNCGYPINPARDLGPRLFTAIAGWGLTVFSAGNGWWWVPVVGPMVGGVVGAAIYFLMIEMHHPENDKNLEDDNSLKDKYELNTVN

SEQ ID NO: 46 - protein, 592 aa, Zygosaccharomyces rouxii
  REGION 1..592: note = Z. rouxii ZYRO0E01210p (Zrou_T5)
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

SEQ ID NO: 47 - protein, 561 aa, Punctularia strigosozonata
  REGION 1..561: note = P. strigosozonata glucoamylase
SSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 48 - protein, 578 aa, Punctularia strigosozonata
  REGION 1..578: note = P. strigosozonata glucoamylase with native signal sequence FGA09
MLSSLIVSGLLASGVCASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 49 - protein, 580 aa, synthetic construct
  REGION 1..580: note = P. strigosozonata glucoamylase with Sc alpha mating factor signalsequence
MRFPSIFTAVLFAASSALASSKSVSAYISSESPIAHSKLLDNIGPDGAKAPGAFPGVVVASPSTDNPNYYYSWIRDSSLVFKTLIDDYVNGKNTSKSLRSLIDDFVTASSVFQQTPNPSGNVSTGGLGEPKFYVNETAFLDSWGRPQRDGPALRSTALITYANYLLDNDNTTWVKDTLWPIIELDVNYVSDFWNYTTFDLWEEVASSSFFTTAVQHRALRQASKLAKTLDKTDNIDSWNTQADNVLCFLQSYWNGSAIIANTGGGRSGIDANTVLASIHTFDSSAGCDATTFQPCSDKALSNLKVYVDAFRSIYEINSGIDPNAAVATGRYPEDVFYDGNPWYLATAAVAEQLYDALYVWNTTGSLEITDISLPFFQQFDSDVKTGTYSDDDTFDSLISSIQSFADGFLEIHAKYTPDDGALSEEFSKTDGSQTSAADLTWSYAAALTAFDARSRDAAVKWGAKGLQVPDGTCKTNEGGDDGLGVPVTFLVKDAETVEGQSVYITGSIATLKSWSPDDALLMSPSDYPTWTLTVNLSASESVQYKYIKKDTAGTVIWESDPNNSLLVPSGGSVTTDDTWR

SEQ ID NO: 50 - DNA, 3050 bp, synthetic construct
  misc_feature 1..3050: note = DNA fragment comprising Sc_ANB1p, Pp_TKL1 gene and Sc_TDH1t
  source 1..3050: mol_type = other DNA
ctactttcgacggaatttgcaatctcccacaatatctccctcctgttctcgtgcagatcggtgaaaacatcttgatcttgctcccgggaattttagattcaggtaggaaattgattacatcaatactgttaccctgaatcatattcgacgatgtcgtctcacacggaaatataattcatttcttggttttccaaaaaaattttcattttttttcacttttttgtttcgtcctcctttttttttttttttttttattttttttcctgtgttcacctttttttttttcagttgacatctttctgcattcttttctgtgtttttttttttttttttcgtttttccattgttcgttcgttgcctgttttttcgccctattgttctcgagcctaaaaattttttcctttcctgctttcctttcttcgttcaaagtttcctattccattgttctctttggtaaactcattgttgtcggaactcagatatattcaggtcaatttactgtacttcaattgacttttttcttgaaatttcaacttgccttttcaacttgttcttcttttttaatcttattctacactttagttcccttaccttgttcctaattattgtctagcaaaaagaaaacatacacctatttcattcacacactaaaacaatgtctgatttgttggccatcaacaccatcagattgttggctgttgacactgttgccaaggctaactctggtcacccaggtgctccaatgggtttggctccagctgctcacactttgttcaagcaaatgagattcaacccaagaaacccagcttggatcaacagagacagatttgttttgtccaatggtcacgcttgtgctttgctatacaccatgttgttcttgtacggttacgactacaccattgatgacttgaaatccttccgtcaattgaactctaagactccaggtcatccagaagctgaattaccaggtgttgaagtcactaccggtccattgggtcaaggtattgctaacgctgtcggtttggctattgctcaagctcaattggctgctacctacaacaagcctaactacgaattattctccaactacacttacgctttcttaggtgacggttgtttgcaagaaggtgttgctcaagaagccatctccttggctggtcacttgggtgatggtttgaaattaattgctttctgggacgacaaccaaatctctattgacggtgacactaacgtctccttcactgaagatgttccagctaagttcagagcccaaggttgggaagtcttgtccgtcaaggacggtaacgacgacttggaaggtatcgctgccgctttggctaaggccaagactaccaacaagccaactttgatcagattgcctaccatcatcggttacggttctttgcaacaaggtactcacggtgttcacggttctccattgaagccagatgatatcaagcaattgaagaagaagtttggtttcgacccagaacaaaactttgttgttccaagagaagttaccgaatcttacgccaagcatgttgctgacaaccaacaagtcgaagttgaatggaacaaattgttgactgcttacaccaaggaatacccagaattgggccaagaattgcacagaagattggacggtaagttgccagaaaactggcaaaaggccttgccaacctacaccgtcgatgacaagccagttgcctctcgtaaattatctgaaatcgtcttaacttccattgaaaaggaattgccagaattagtcggtggttctgctgacttgaccggttccaacttgactagatggccagatgctgtcgacttccaaccaaagtctaccggtttaggtgacttctccggtcgttatttcagatttggtgttagagaacacggtatgggtgccattattaacggtatctctgcctacggtgctaacttcaaggcttacggtggtactttcttgaactttgtttcctacgcttctggtgctgttagattgtccgctttgtccggtcatccaatcatctgggttgctactcacgattccatcggtttgggtgaagacggtccaactcaccaaccaatcgaaactttggctcacttaagagctttgccaaacttgatggtctggcgtccagctgatggtaatgaaacctctgctgcctacttgagagccattgaatccaagcacactccatctatcatcgctttgaccagacaaaacttgcctcaattggaacactcttctatcgaaaaagctgccaagggtggttacaccgtctacccagttgaaaaccctgatattattttggttgcttctggttctgaagtttccattgccattgatggtgccaagaagttgggtaccgaaggtgtcaaggcttctgttgtctccatcccagatttcttcaccttcgactctcaagctagatcttaccaattgtctgttttgccagatggtgtcccaatcatgtccgtcgaagttatgtctactttcggttggtctaagtactcccacgaacaattcggtattaacagattcggaatttctggtccaggtccagaaatctacaaattcttcgaattcaccgctgaaggtgtcgctgacagagcttctaaggttgtccaattctacaagggtaaggaactattatctccattaaacaaggctttcgaatccgtccacgcgtaaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctccttccaactgtcctgtgcatatccgcccttgctgctcctttcgatatctt

SEQ ID NO: 51 - DNA, 2717 bp, synthetic construct
  misc_feature 1..2717: note = DNA fragment comprising Sc_MYO4p, Sc_DAK1 and Sc_GPM1t
  source 1..2717: mol_type = other DNA
atccgtacacgtttgtgccgaacccgaagtattgcaacaggtcgaagttcgtgcataacaatagaacaatagctttttgaccttccccttattttatttcaaaaggtaacagttagggctattcttaaactttctttagggtacacaatgaaaacatcaagattgagttaaaacccttaaaataaaactaggtctttaagaaactcactctccgggttatccatagaatgtttaccattctttagggttctgaactaatgcgaaaaaaaaaaaaggaaaagactgaaaaattcgaaaatatctcgaagtttagcagtagtagatgagaatagggtgtcttttatcgaaaacctgcaattgtaatacaacctttttttgaaagtcagcctttattctgattctgcatactcaattcctcaattcctacggagttatcacctttttttttaccttctcttcttttcttattgttctagctgaaaacattgttaccagtttggcgagacaatttattttcaatacgatacccttttgttcttctttttataattcaatctaattctaaaacacaaaaaaacaaaaaaaatcctataaccagttctcccgaatgtccgctaaatctttcgaagttaccgacccagtcaactcttctttgaagggttttgctttggccaacccatccattactttggtcccagaagaaaagatcttattcagaaagactgactctgacaaaattgctttgatctccggtggtggttccggtcacgaaccaacccacgctggtttcatcggtaagggtatgttgtccggtgctgtcgttggtgaaatctttgcttctccatccaccaagcaaatcttgaatgctatcagattagtcaacgaaaacgcttctggtgtcttgttgattgtcaagaactacactggtgacgtcttgcatttcggtttatctgctgaaagagctagagctttgggtattaactgtagagttgccgtcatcggtgacgatgttgctgtcggtcgtgaaaagggtggtatggttggtagacgtgctttggctggtactgtcttggttcacaagattgttggtgctttcgctgaagaatactcctccaagtacggtttagatggtactgctaaggttgccaagatcatcaacgacaacttggttaccatcggttcttctttggaccactgtaaggttccaggtagaaagttcgaatctgaattgaacgaaaagcaaatggaattgggtatgggtatccacaacgaaccaggtgttaaggtcttggacccaattccatccactgaagatttgatttccaaatacatgttgccaaagttgctagacccaaacgacaaggacagagctttcgttaagttcgatgaagatgacgaagttgttttgttggtcaacaacttgggtggtgtttctaacttcgtcatctcttctattacctccaagaccaccgatttcttaaaggaaaactacaacatcactccagtccaaaccattgccggtactttgatgacctctttcaacggtaacggtttctccatcaccttgttgaatgccaccaaagctaccaaggctttgcaatctgatttcgaagaaatcaaatccgtcttagatttgttgaacgccttcaccaacgccccaggttggccaattgctgacttcgaaaagacctctgctccatctgttaacgatgacttgttgcacaacgaagttactgccaaggccgtcggtacttacgatttcgacaaattcgctgaatggatgaagtctggtgctgaacaagtcatcaaatctgaaccacacatcactgaattggacaaccaagttggtgatggtgactgtggttacactttggttgctggtgtcaagggtatcactgaaaacttggacaaattgtccaaggactctttgtctcaagctgttgctcaaatttctgatttcattgaaggttccatgggtggtacttctggtggtttgtactccatcttgttgtctggtttctcccacggtttgatccaagtttgtaagtccaaggatgaacctgtcaccaaggaaattgttgccaagtctctaggtattgctttggacactttatacaagtacaccaaggccagaaagggttcttccaccatgatcgatgctttggaaccatttgtcaaggaattcactgcttctaaggacttcaacaaggctgttaaggctgctgaagaaggtgccaagtccactgctactttcgaagctaagttcggtagagcttcttacgttggtgactcttctcaagttgaagatccaggtgctgttggtttatgtgaattcttgaagggtgtccaatctgcgctttaaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatacctcacccgatcccgaagaaagtccaactccagttcgattccctgctttcgagt

SEQ ID NO: 52 - DNA, 1837 bp, synthetic construct
  misc_feature 1..1837: note = DNA fragment comprising Sc_HHF2p, Ec_gldA and Sc_EFM1t
  source 1..1837: mol_type = other DNA
acccgatcccgaagaaagtccaactccagttcgattccctgctttcgagtgtgcacaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctcaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaataatggacagaatcatccaatctccaggtaagtacatccaaggtgctgatgttatcaacagattaggtgaatacttgaagccattggctgaaagatggttagtcgtcggtgacaaattcgttttgggtttcgctcaatccaccgtcgaaaagtctttcaaggatgctggtttggttgttgaaatcgctccattcggtggtgaatgttctcaaaatgaaattgaccgtttgagaggtattgctgaaactgctcaatgtggtgccatcttgggtattggtggtggtaagactttggacactgccaaggctttggcccacttcatgggtgttccagttgccattgctccaaccattgcttctaccgatgctccatgttctgctttgtccgttatctacaccgacgaaggtgaatttgaccgttacttgttgttgccaaacaacccaaacatggtcattgtcgacaccaagatcgttgccggtgctccagccagattattggctgccggtatcggtgatgctttggctacctggttcgaagccagagcttgttccagatctggtgctactaccatggccggtggtaaatgtactcaagctgctttagctttggctgaattgtgttacaacactttgttggaagaaggcgaaaaggctatgttggctgctgaacaacacgttgttactccagctttggaaagagtcattgaagccaacacctacttgtccggtgttggattcgaatctggtggtttagctgccgctcatgccgtccacaacggtttgactgccatcccagatgctcaccactactaccacggtgaaaaggttgctttcggtactttgactcaattagtcttggaaaacgctccagtcgaagaaatcgaaaccgttgctgctctatcccacgctgtcggtttgcctatcactttggctcaattggacatcaaggaagatgtcccagctaagatgagaattgttgctgaagctgcttgtgctgaaggtgaaaccattcacaacatgccaggtggtgccaccccagaccaagtctacgctgctttgttggttgctgaccaatacggtcaaagattcttgcaagaatgggagtaaatttgatctgtagcctaagtataaaattctacgtatgtatatatttacatgcaattttttctttttccaattcatgcctctcctcggatcggccagttgggagcaaggtggtacttctgtcgtcgtattt

SEQ ID NO: 53 - DNA, 2789 bp, synthetic construct
  misc_feature 1..2789: note = DNA fragment comprising Sc_ANB1p0001, ZYRO0E01210, Zrou_T5,Sc_TEF1t0001
  source 1..2789: mol_type = other DNA
tcctcggatcggccagttgggagcaaggtggtacttctgtcgtcgtatttgtgcagatcggtgaaaacatcttgatcttgctcccgggaattttagattcaggtaggaaattgattacatcaatactgttaccctgaatcatattcgacgatgtcgtctcacacggaaatataattcatttcttggttttccaaaaaaattttcattttttttcacttttttgtttcgtcctcctttttttttttttttttttattttttttcctgtgttcacctttttttttttcagttgacatctttctgcattcttttctgtgtttttttttttttttttcgtttttccattgttcgttcgttgcctgttttttcgccctattgttctcgagcctaaaaattttttcctttcctgctttcctttcttcgttcaaagtttcctattccattgttctctttggtaaactcattgttgtcggaactcagatatattcaggtcaatttactgtacttcaattgacttttttcttgaaatttcaacttgccttttcaacttgttcttcttttttaatcttattctacactttagttcccttaccttgttcctaattattgtctagcaaaaagaaaacatacacctatttcattcacacactaaaacaatgggtaagagaactcaaggtttcatggactacgttttctccagaacttctactgctggtttgaaaggtgccagattgagatacactgctgctgccgttgctgttatcggtttcgctttattcggttacgaccaaggtttgatgtctggtttgattaccggtgaccaattcaacaaggaattccctccaaccaaatctaacggtgacaacgacagatacgcctccgtcatccaaggtgctgtcactgcttgttacgaaattggttgtttcttcggttctttgttcgtcttattctttggtgatgccatcggtagaaagccattaatcattttcggtgccatcatcgtcatcattggtactgttatctccactgctccattccaccacgcttggggtttgggtcaatttgttgtcggtagagttatcactggtgtcggtactggtttcaacacttccaccatcccagtctggcaatctgaaatgaccaagccaaacatcagaggtgctatgattaacttggatggttccgtcattgctttcggtactatgattgcttactggttggatttcggtttctctttcatcaactcttctgttcaatggagattcccagtttccgttcaaatcatctttgctttggtcttattgttcggtattgtcagaatgccagaatctccaagatggttgatggccaagaagcgtccagctgaagccagatacgttttggcttgtttgaatgacttgccagaaaacgacgatgccatcttggccgaaatgacttctttgcacgaagctgtcaacagatcttctaaccaaaagtctcaaatgaaatctttgttctccatgggtaagcaacaaaacttctccagagctttgattgcttcctctacccaattcttccaacaattcaccggttgtaatgctgccatctactactccactgttttgttccaaaccaccgttcaattagatagattgttggctatgatcttgggtggtgtctttgctaccgtttacaccttgtccactttgccatctttctacttggttgaaaaggttggtcgtcgtaagatgttcttcttcggtgctttaggtcaaggtatttctttcattattacctttgcttgtttggtcaacccaaccaagcaaaacgctaagggtgctgctgtcggtttatacttgttcatcatctgtttcggtttggctatcttggaattgccatggatctacccaccagaaattgcttccatgagagtcagagctgctaccaacgctatgtccacctgtaccaactgggttaccaactttgccgttgtcatgttcaccccagtctttatccaaacttctcaatggggttgttacttgttcttcgctgtcatgaacttcatctacttgcctgttatcttcttcttctacccagaaactgccggtcgttctttggaagaaatcgatattattttcgctaaggctcatgttgacggtactctaccatggatggttgctcacagattaccaaagttgtccatgactgaagtcgaagattactctcaatctttgggtttgcacgacgatgaaaacgaaaaggaagaatacgacgaaaaagaagctgaagctaacgctgccttgttccaagttgaaacctcttccaagtctccatcctccaacagaaaggatgacgacgctccaattgaacacaacgaagttcaagaatctaacgacaactcctctaactcttctaacgttgaagctccaattccagttcaccacaacgacccataaaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttcctcatttggaacagcaggcgaggtgtattggtgcgagaagaggtgtttccgag

SEQ ID NO: 54 - DNA, 1576 bp, synthetic construct
  misc_feature 1..1576: note = DNA fragment comprising Sc_PFY1p, Ec_gldA and Sc_EFM1t
  source 1..1576: mol_type = other DNA
acccgatcccgaagaaagtccaactccagttcgattccctgctttcgagtgtgcaggagacgttactttgtttatatatattagtatgtacaatcgcaaagaaatggagtgatgacatgttgtagtatttagtatgaggttactgtgtgggaggttttaccatgatttttggcgagaacacgccatgaaatgtctttgtacgaaactcattacccgcattaatattttttttctttttaaagctcagttgaccctttctcattcccttcttaaaacaactgtgtgatccttgagaaaagataaattacatacacaacataaacccaactacgatcgcaaataatggacagaatcatccaatctccaggtaagtacatccaaggtgctgatgttatcaacagattaggtgaatacttgaagccattggctgaaagatggttagtcgtcggtgacaaattcgttttgggtttcgctcaatccaccgtcgaaaagtctttcaaggatgctggtttggttgttgaaatcgctccattcggtggtgaatgttctcaaaatgaaattgaccgtttgagaggtattgctgaaactgctcaatgtggtgccatcttgggtattggtggtggtaagactttggacactgccaaggctttggcccacttcatgggtgttccagttgccattgctccaaccattgcttctaccgatgctccatgttctgctttgtccgttatctacaccgacgaaggtgaatttgaccgttacttgttgttgccaaacaacccaaacatggtcattgtcgacaccaagatcgttgccggtgctccagccagattattggctgccggtatcggtgatgctttggctacctggttcgaagccagagcttgttccagatctggtgctactaccatggccggtggtaaatgtactcaagctgctttagctttggctgaattgtgttacaacactttgttggaagaaggcgaaaaggctatgttggctgctgaacaacacgttgttactccagctttggaaagagtcattgaagccaacacctacttgtccggtgttggattcgaatctggtggtttagctgccgctcatgccgtccacaacggtttgactgccatcccagatgctcaccactactaccacggtgaaaaggttgctttcggtactttgactcaattagtcttggaaaacgctccagtcgaagaaatcgaaaccgttgctgctctatcccacgctgtcggtttgcctatcactttggctcaattggacatcaaggaagatgtcccagctaagatgagaattgttgctgaagctgcttgtgctgaaggtgaaaccattcacaacatgccaggtggtgccaccccagaccaagtctacgctgctttgttggttgctgaccaatacggtcaaagattcttgcaagaatgggagtaaatttgatctgtagcctaagtataaaattctacgtatgtatatatttacatgcaattttttctttttccaattcatgcctctcctcggatcggccagttgggagcaaggtggtacttctgtcgtcgtattt

SEQ ID NO: 55 - DNA, 500 bp, synthetic construct
  misc_feature 1..500: note = Repair DNA fragment for homologous recombination and deletingGPD2
  source 1..500: mol_type = other DNA
atgcaagcgtgtatcttctaagattcagtcatcatcattaccgagtttgttttccttcacatgatgaagaaggtttgagtatgctcgaaacaataagacgacgatggctctgccattgttatattacgcttttgcggcgaggtgccgatgggttgctgaggggaagagtgtttagcttacggacctattgccattgttattccgattaatctattgttcagcagctcttctctaccctgtcattctagtataagaatctatcattaacgttaatttctatatatacataactaccattatacacgctattatcgtttacatatcacatcaccgttaatgaaagatacgacaccctgtacactaacacaattaaataatcgccataaccttttctgttatctatagcccttaaagctgtttcttcgagctttttcactgcagtaattctccacatgggcccagccactgagataagagcgctatgttagtcactactgacg

SEQ ID NO: 56 - DNA, 322 bp, Saccharomyces cerevisiae
  misc_feature 1..322: note = EBO1 terminator (Sc_ENO1t)
  source 1..322: mol_type = genomic DNA
ggtctcgtaaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctcggagacc

SEQ ID NO: 57 - DNA, 621 bp, Saccharomyces cerevisiae
  misc_feature 1..621: note = S. cerevisiae ENO2 promoter
  source 1..621: mol_type = genomic DNA
ggtctcggtgcgtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataataatgggagacc

SEQ ID NO: 58 - DNA, 422 bp, Saccharomyces cerevisiae
  misc_feature 1..422: note = S. cerevisiae YOX1 terminator (Sc_YOX1t)
  source 1..422: mol_type = genomic DNA
ggtctcgtaaatctaatctctagtcattatttattcgcaaattcatttccctatacggcattcatacatatcattgttcacttcagtcctagcatatatcataaaatatacaattgttttctaattaccttacgttttttaaaagacttctataatacctcttttaactttacatgtagtcaaaataaagtgcagttccatcgatggtactttctcaccccggttgagtgatgttaacgatgtttaccgtataaaacttaattatattatatctttttttgcttatatgttatacatagaataaaaagttgattaaacacacattggtctgaaaacacgtgtagtactttctcctttgaagaataaaaaaagaaaataaagataataaaaacgaaaatagcgtacaattatcctcggagacc

SEQ ID NO: 59 - DNA, 20 bp, synthetic construct
  misc_feature 1..20: note = INT14.02 protospacer
  source 1..20: mol_type = other DNA
ggagagaaaggcccgggcgt

SEQ ID NO: 60 - DNA, 178 bp, Saccharomyces cerevisiae
  misc_feature 1..178: note = Sc_INT14.02_FLANK5
  source 1..178: mol_type = genomic DNA
tttgagcggacgccgaaacgttttttttttttgctttagtccagatactaaccttttataattttctttttagggaggaagaccggtctaagctcttagaggttctcgcatacccaagtaaaagccaagaccgaagcaaacacgcaggataatttttcggtttactctcactgcagct

SEQ ID NO: 61 - DNA, 571 bp, Saccharomyces cerevisiae
  misc_feature 1..571: note = Sc_INT14.02_FLANK3
  source 1..571: mol_type = genomic DNA
catcttgatttatgtcttgtttgaggagatttcggataaatattcttcgaggggaaaatgtcgtaaaaataaaaacaataaagaaacaaaacgattaaaagaaaaactcaaccatgctcaaaagtccttctcttttttttcacgtgctgcgctgatgtaagcagcaggacatggcaggaagaaaatcggagaattgtggccgcgacacctgacaaacaatcgtcaggcaaatgtaaaattgtacatgaaaggctgcgtagtcaatgtcttagaaggccggcccgcatgatccagaagcagaccataaaaaaatcaaaaattcgcggtctggcagcaggcataagatgcatagcgttatcctaaatgacatcacgatgataaatcctccgccgcatgatgcttttgatttgcctaagggcctgccatcgtgctggctcaaactattgagggtctacataccttgaaaatccaagtaaaaggatggatatcgttatactaaaagcaacacagaaaaggtccacgtcagttccacacaataacatttacgtagtgttcatgccgaagcagttac

SEQ ID NO: 62 - DNA, 20 bp, synthetic construct
  misc_feature 1..20: note = INT95 protospacer
  source 1..20: mol_type = other DNA
gaaggcctatggaaatggcc

SEQ ID NO: 63 - DNA, 150 bp, Saccharomyces cerevisiae
  misc_feature 1..150: note = Sc_INT95B_FLANK5
  source 1..150: mol_type = unassigned DNA
atagttacagctaaacatttgcccaaaaccatcttcttatatatatatctgcgatggcgagcccagcggaagggatgtccgcttactaattccgacacacctactttcgacggaatttgcaatctcccacaatatctccctcctgttctc

SEQ ID NO: 64 - DNA, 150 bp, Saccharomyces cerevisiae
  misc_feature 1..150: note = Sc_INT95B_FLANK3
  source 1..150: mol_type = unassigned DNA
cttccaactgtcctgtgcatatccgcccttgctgctcctttcgatatcttgatgccgataattttcacatggatgagtcgcagtgtcgtcgtacgtgatgagggcaggcttgaaaattattcttcgagcccaattctctttacttatagg

SEQ ID NO: 65 - DNA, 20 bp, synthetic construct
  misc_feature 1..20: note = GPD2 protospacer
  source 1..20: mol_type = other DNA
gatcttttacactccatcaa